SEQUENCE LISTING (51 sequences)

SEQ ID NO: 1 - protein, 931 aa, Homo sapiens [sequence omitted]

SEQ ID NO: 2 - DNA, 2796 bp, Homo sapiens
  misc_feature 1..2796: note = RAG-1 Accession number: BC037344.2
  source 1..2796: mol_type = genomic DNA
atggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctgggctatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtatagaaattaa

SEQ ID NO: 3 - protein, 1040 aa, Mus musculus
  REGION 1..1040: note = MISC_FEATURE - RAG-1 Accession number: NP_033045.2
MAASLPSTLSFSSAPDEIQHPQIKFSEWKFKLFRVRSFEKAPEEAQKEKDSSEGKPYLEQSPVVPEKPGGQNSILTQRALKLHPKFSKKFHADGKSSDKAVHQARLRHFCRICGNRFKSDGHSRRYPVHGPVDAKTQSLFRKKEKRVTSWPDLIARIFRIDVKADVDSIHPTEFCHDCWSIMHRKFSSSHSQVYFPRKVTVEWHPHTPSCDICFTAHRGLKRKRHQPNVQLSKKLKTVLNHARRDRRKRTQARVSSKEVLKKISNCSKIHLSTKLLAVDFPAHFVKSISCQICEHILADPVETSCKHLFCRICILRCLKVMGSYCPSCRYPCFPTDLESPVKSFLNILNSLMVKCPAQDCNEEVSLEKYNHHVSSHKESKETLVHINKGGRPRQHLLSLTRRAQKHRLRELKIQVKEFADKEEGGDVKAVCLTLFLLALRARNEHRQADELEAIMQGRGSGLQPAVCLAIRVNTFLSCSQYHKMYRTVKAITGRQIFQPLHALRNAEKVLLPGYHPFEWQPPLKNVSSRTDVGIIDGLSGLASSVDEYPVDTIAKRFRYDSALVSALMDMEEDILEGMRSQDLDDYLNGPFTVVVKESCDGMGDVSEKHGSGPAVPEKAVRFSFTVMRITIEHGSQNVKVFEEPKPNSELCCKPLCLMLADESDHETLTAILSPLIAEREAMKSSELTLEMGGIPRTFKFIFRGTGYDEKLVREVEGLEASGSVYICTLCDTTRLEASQNLVFHSITRSHAENLQRYEVWRSNPYHESVEELRDRVKGVSAKPFIETVPSIDALHCDIGNAAEFYKIFQLEIGEVYKHPNASKEERKRWQATLDKHLRKRMNLKPIMRMNGNFARKLMTQETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKSSGFTMNSKETLGDPLGIEDSLESQDSMEF

SEQ ID NO: 4 - DNA, 3123 bp, Mus musculus
  misc_feature 1..3123: note = RAG-1 Accession number: NM_009019.2
  source 1..3123: mol_type = genomic DNA
atggctgcctccttgccgtctaccctgagcttcagttctgcacccgatgaaattcaacacccacaaatcaaattttccgagtggaaatttaagctgtttagggtgagatcctttgaaaaggcacccgaagaagcacagaaggagaaggattcctcagaggggaaaccttacctagaacagtctccagtagttccagagaagcctggtggtcagaactcaattctgactcaacgagcactgaaactccatcctaaattttcaaagaaattccatgctgatgggaagtcaagcgacaaagcagttcaccaagccaggcttagacacttctgccgcatctgtgggaatcgtttcaagagtgacgggcacagccggagatacccagtccacgggcccgtggacgctaaaacccaaagtcttttccgaaagaaggaaaaaagagtcacttcctggccagacctcattgccaggattttccggatcgacgtgaaggcagatgttgactccatccacccgacggaattctgccatgactgttggagcatcatgcacagaaagttcagcagttcccacagtcaggtctacttcccaaggaaagtgaccgtggagtggcacccccacacaccgtcctgtgacatctgttttactgcccatcggggactcaagaggaagagacatcagcccaatgtgcagctcagcaagaaactaaaaactgtgctcaaccacgcgagacgggaccgtcgcaagagaactcaggctagggtcagcagcaaggaagtcctgaagaagatctccaactgcagtaagattcatctcagtaccaagcttcttgccgtggacttcccagcacactttgtgaaatccatctcctgccagatatgcgaacacattctggctgatcccgtggagaccagctgcaagcatctattctgtaggatctgcattctcagatgtctcaaagtcatgggcagctattgtccctcttgccgatatccgtgcttccctactgacctggagagcccagtgaagtcctttctgaacatcttgaattctctcatggtcaagtgtcccgcgcaagattgcaatgaggaagtgagtctggaaaaatataaccaccatgtgtcaagccacaaagaatctaaagagactttggtgcatatcaataaagggggacggcctcgccagcatctcctgtcactgacgagaagggcgcagaaacatcggctgagggagctcaagattcaagtcaaagaatttgctgacaaagaagaaggtggagatgtgaaggctgtctgcttgacattgtttctcctggcactgagggcgaggaatgagcacaggcaagctgatgaattagaggccatcatgcaaggcaggggctccgggcttcaaccagctgtttgcttggccatccgtgtcaataccttcctcagctgtagccaataccataagatgtacaggactgtgaaagctatcactgggaggcagatttttcaacctttgcatgctcttcggaatgccgagaaagtccttctgccaggctaccatccctttgagtggcagcccccactgaagaatgtgtcctccagaactgatgttggaattattgatgggctgtctggacttgcctcctctgtggatgagtacccagtagataccattgcgaagaggttccgctacgactctgctttggtgtctgctttgatggacatggaagaagacatcttggaaggcatgagatcccaagatcttgatgactacctgaatggtcccttcacagtggtggtaaaggagtcttgcgatggaatgggggatgtgagtgagaagcacgggagtgggcccgcagttccagaaaaggccgttcgtttctctttcacagtcatgagaattacgatagagcatggttcacagaacgtgaaggtgtttgaggaacccaagcccaattctgaactgtgttgcaagccgttgtgtcttatgctggcagatgagtctgaccatgagacccttactgctattctaagccccctcattgccgagagggaggccatgaagagcagtgaattaacgctggagatgggaggcatccccaggacttttaaattcatcttcaggggcactggatacgatgaaaaacttgtccgggaagtagaaggcttggaagcttctggctcagtctacatctgtacactctgtgacaccacccgtttggaagcctctcagaatcttgtcttccactccataaccagaagccacgccgagaacctgcagcgctatgaggtctggcggtccaatccgtatcatgagtccgtggaagagctccgggaccgggtgaaaggggtctctgccaaacctttcatcgagacagtcccttccatagatgcgcttcactgtgacattggcaatgcagctgaattctataagattttccagctggagataggggaagtgtataaacatcccaatgcctctaaagaggaaaggaagagatggcaggccacgctggacaaacatctccggaaaaggatgaacttaaaaccaatcatgaggatgaatggcaactttgcccggaagcttatgacccaagagactgtagacgcagtttgtgagttaattccttctgaggagaggcatgaagctctcagggagctcatggacctttacctgaagatgaaacccgtgtggcgctcttcatgtcccgctaaagagtgtccagagtccctctgtcagtacagtttcaactcacagcgtttcgcggaactcctctccaccaagttcaaatatagatacgagggcaaaatcaccaattactttcacaaaaccttggcacatgtccctgaaattattgaaagggatggctctatcggggcctgggcaagtgagggaaatgaatcgggtaacaagctgtttagacggtttcggaaaatgaatgccaggcagtccaagtgctatgagatggaagatgtcctgaaacatcactggctgtatacttcaaaatacctccagaagtttatgaatgctcataacgcgttaaaaagctctgggtttaccatgaactcaaaggagaccttaggggaccctttgggcattgaggactctctggaaagccaagattcaatggagttttaa

SEQ ID NO: 5 - protein, 527 aa, Homo sapiens
  REGION 1..527: note = MISC_FEATURE - RAG-2 Accession number: NP_001230715.1
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 6 - DNA, 1584 bp, Homo sapiens
  misc_feature 1..1584: note = RAG-2 Accession number: NM_001243786.1
  source 1..1584: mol_type = genomic DNA
atgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattag

SEQ ID NO: 7 - protein, 527 aa, Mus musculus
  REGION 1..527: note = MISC_FEATURE - RAG-2 Accession number: NP_033046.1
MSLQMVTVGHNIALIQPGFSLMNFDGQVFFFGQKGWPKRSCPTGVFHFDIKQNHLKLKPAIFSKDSCYLPPLRYPATCSYKGSIDSDKHQYIIHGGKTPNNELSDKIYIMSVACKNNKKVTFRCTEKDLVGDVPEPRYGHSIDVVYSRGKSMGVLFGGRSYMPSTQRTTEKWNSVADCLPHVFLIDFEFGCATSYILPELQDGLSFHVSIARNDTVYILGGHSLASNIRPANLYRIRVDLPLGTPAVNCTVLPGGISVSSAILTQTNNDEFVIVGGYQLENQKRMVCSLVSLGDNTIEISEMETPDWTSDIKHSKIWFGSNMGNGTIFLGIPGDNKQAMSEAFYFYTLRCSEEDLSEDQKIVSNSQTSTEDPGDSTPFEDSEEFCFSAEATSFDGDDEFDTYNEDDEDDESVTGYWITCCPTCDVDINTWVPFYSTELNKPAMIYCSHGDGHWVHAQCMDLEERTLIHLSEGSNKYYCNEHVQIARALQTPKRNPPLQKPPMKSLHKKGSGKVLTPAKKSFLRRLFD

SEQ ID NO: 8 - DNA, 1584 bp, Mus musculus
  misc_feature 1..1584: note = RAG-2 Accession number: NM_009020.3
  source 1..1584: mol_type = genomic DNA
atgtccctgcagatggtaacagtgggtcataacatagccttaattcaaccaggcttctcacttatgaattttgatggccaagttttcttctttggccagaaaggctggcctaagagatcctgtcctactggagtctttcattttgatataaaacaaaatcatctcaaactgaagcctgcaatcttctctaaagattcctgctacctcccacctcttcgttatccagctacttgctcatacaaaggcagcatagactctgacaagcatcaatatatcattcacggagggaaaacaccaaacaatgagctttccgataagatttatatcatgtctgtcgcttgcaagaataacaaaaaagttactttccgttgcacagagaaagacttagtaggagatgtccctgaacccagatacggccattccattgacgtggtgtatagtcgagggaaaagcatgggtgttctctttggaggacgttcatacatgccttctacccagagaaccacagaaaaatggaatagtgtagctgactgcctaccccatgttttcttgatagattttgaatttgggtgtgctacatcatatattctcccagaacttcaggatgggctgtcttttcatgtttctattgccagaaacgataccgtttatattttgggaggacactcacttgccagtaatatacgccctgctaacttgtatagaataagagtggaccttcccctgggtaccccagcagtgaattgcacagtcttgccaggaggaatctctgtctccagtgcaatcctcactcaaacaaacaatgatgaatttgttattgtgggtggttatcagctggaaaatcagaaaaggatggtctgcagccttgtctctctaggggacaacacgattgaaatcagtgagatggagactcctgactggacctcagatattaagcatagcaaaatatggtttggaagcaacatgggaaacgggactattttccttggcataccaggagacaataagcaggctatgtcagaagcattctatttctatactttgagatgctctgaagaggatttgagtgaagatcagaaaattgtctccaacagtcagacatcaacagaagatcctggggactccactccctttgaagactcagaggaattttgtttcagtgctgaagcaaccagttttgatggtgacgatgaatttgacacctacaatgaagatgatgaagatgacgagtctgtaaccggctactggataacatgttgccctacttgtgatgttgacatcaatacctgggttccgttctattcaacggagctcaataaacccgccatgatctattgttctcatggggatgggcactgggtacatgcccagtgcatggatttggaagaacgcacactcatccacttgtcagaaggaagcaacaagtattattgcaatgaacatgtacagatagcaagagcattgcaaactcccaaaagaaacccccccttacaaaaacctccaatgaaatccctccacaaaaaaggctctgggaaagtcttgactcctgccaagaaatccttccttagaagactgtttgattaa

SEQ ID NO: 9 - protein, 29 aa, synthetic construct
  REGION 1..29: note = protein stabilizing sequence
MKKLKLRLTHLWYKLLMKLGLKSDEVYYI

SEQ ID NO: 10 - protein, 489 aa, synthetic construct
  REGION 1..489: note = anti CAR-CD19
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 789 bp, synthetic construct
  misc_feature 1..789: note = anti Tn-MUC1 CAR
  source 1..789: mol_type = other DNA
atggaatggtcttgggtgttcctgttcttcctgagcgtgaccaccggcgtgcacagccaggtgcagctgcagcagtctgatgccgagctcgtgaagcctggcagcagcgtgaagatcagctgcaaggccagcggctacaccttcaccgaccacgccatccactgggtcaagcagaagcctgagcagggcctggaatggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggcggaggcggatctggcggcggaggatctgggggaggcggctctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaag

SEQ ID NO: 12 - protein, 220 aa, synthetic construct
  REGION 1..220: note = NY-ESO-1-specific TCR
EVQLLESGGGLVQPGGSLRLSCAASGFTFSAYGMGWVRQAPGKGLEWVSSIGSSGGGTAYADSVKGRFTISRDNSKNTLYLQMNSLRAEDTAVYYCAGELLPYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 13 - protein, 226 aa, synthetic construct
  REGION 1..226: note = 3BNC117 (anti-HIV Antibody)
QVQLLQSGAAVTKPGASVRVSCEASGYNIRDYFIHWWRQAPGQGLQWVGWINPKTGQPNNPRQFQGRVSLTRHASWDFDTYSFYMDLKALRSDDTAVYFCARQRSDYWDFDVWGSGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 224 aa, synthetic construct
  REGION 1..224: note = Adalimumab (Humira ) an anti-TNF? Antibody
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 116 aa, synthetic construct
  REGION 1..116: note = PankoMab-GEX an anti MUC1 glycoform Antibody heavy chain
EVQLVESGGGLVQPGGSMRLSCVASGFPFSNYWMNWVRQAPGKGLEWVGEIRLKSNNYTTHYAESVKGRFTISRDSKNSLYLQMNSLKTEDTAVYYKTRHYYFDYWGQGTLVTVSS

SEQ ID NO: 16 - protein, 114 aa, synthetic construct
  REGION 1..114: note = PankoMab-GEX an anti MUC1 glycoform Antibody light chain
DIVMTQSPLSNPVTPGEPASISCRSSKSLLHANGITYFFWYLQKPGQSPQLLIYQMSNLASGVPDRFSGSGSGTDFTLRISRVEAEDVGVYYCAQNLELPPTFGQGTKVEIKRA

SEQ ID NO: 17 - DNA, 1200 bp, Homo sapiens
  misc_feature 1..1200: note = iCasp9
  source 1..1200: mol_type = genomic DNA
atgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 18 - DNA, 23 bp, synthetic construct
  misc_feature 1..23: note = miR-181a recognition element
  source 1..23: mol_type = other DNA
actcaccgacagcgttgaatgtt

SEQ ID NO: 19 - DNA, 23 bp, synthetic construct
  misc_feature 1..23: note = ECMV IRES R1
  source 1..23: mol_type = other DNA
catatagacaaacgcacaccggc

SEQ ID NO: 20 - DNA, 35 bp, synthetic construct
  misc_feature 1..35: note = IgKVm-rec-F1
  source 1..35: mol_type = other DNA
gtccctgccaggttyagtggcagtggrtcwrggac

SEQ ID NO: 21 - DNA, 33 bp, synthetic construct
  misc_feature 1..33: note = mHA-SbfI-F1 primer
  source 1..33: mol_type = other DNA
cttacccctatgatgtgcctgactacgcttgca

SEQ ID NO: 22 - DNA, 20 bp, synthetic construct
  misc_feature 1..20: note = IgKCm-rec-Rev2 primer
  source 1..20: mol_type = other DNA
ggatggtggaagatggatac

SEQ ID NO: 23 - DNA, 2529 bp, synthetic construct
  misc_feature 1..2529: note = Anti-polio BCR
  source 1..2529: mol_type = other DNA
atgaagctgagagtgggccagacactgggcacaatccctagacagtgcgaggtgctgctgctcctgctgcttctgggacttgtggatggagtgtcactgtccccaggacagacagccaacatcgcctgctctggagataaattggcggataaatatattaactggtatcagcagaagtcaggccagtcccctgttctggtcatctatgaagatgcgaagcggccctcagggatccctgagcgattctctggctccagctctgggaacacagccactctaaccatcagcgggacccaggctatggatgaggctgactactactgtcaggcgtgggacaacagcgctggatatgtattcggaactgggaccaaagtcaccgtcggtcagcccaagtccactcccacactcaccatgtttccaccttcccctgaggagctccaggaaaacaaagccacactcgtgtgtctgatttccaatttttccccaagtggtgtgacagtggcctggaaggcaaatggtacacctatcacccagggtgtggacacttcaaatcccaccaaagaggacaacaagtacatggccagcagcttcttacatttgacatcggaccagtggagatctcacaacagttttacctgccaagttacacatgaaggggacactgtggagaagagtctgtctcctgcagaatgtctcggcagcggcgccacaaacttctctctgctaaagcaagcaggtgatgttgaagaaaaccccgggcctatggagtttgggctgagctgggttttcctcgttgctctttttagaggtgtccagtgtctcgagtcgggcccaggactggtgaagccttcggagaccctgtccctcacctgcagtgtctctagtggcttcatcagtctttattcgtggagttggatccgccaggcaccagggacgggactggagtggattgggtccatccgctatagtgagaccgcctattacaacccgtccctcaggggtcgagtcttcatatcactagacacgtccaagaatcacttctccctgaagatgagttctctgaccgccacagatacggccgtttattattgtgcgcgagactacagtaagagtggctacgatcgttcggggcggttcgacccctggggccagggtaccccagtcatcgtctccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacaaagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaaatatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactgagggggaggtgaatgctgaggaggaaggctttgagaacctgtggaccactgcctccaccttcatcgtcctcttcctcctgagcctcttctacagcaccaccgtcaccctgttcaaggtgaaatga

SEQ ID NO: 24 - DNA, 2214 bp, synthetic construct
  misc_feature 1..2214: note = anti-RSV BCR
  source 1..2214: mol_type = other DNA
atggcgacgggttcaagaacttccctacttcttgcatttggcctgctttgtttgccgtggttacaggagggctcggcagacatccagatgacacagagcccctctacactgagcgccagcgtgggagacagagtgaccatcacatgcaagtgccagctgagcgtgggctacatgcactggtatcagcagaagcctggcaaggcccctaagctgctgatctacgacacaagcaagctggcctctggcgtgccctctagattttctggcagcggctctggcaccgagttcaccctgacaatctctagcctgcagcctgacgacttcgccacctactactgcttccaaggctctggctaccccttcacattcggcggaggcaccaagctggaaatcaaggtggccgctcccagcgtgttcatcttccctccctctgatgaacagctgaaaagcggaacagccagcgtggtgtgtctgctgaacaacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgccgcgcgaaacgcggaagcggagctactaacttcagcctgctgaagcaggctggagacgtggaggagaaccctggacctatggcgacgggttcaagaacttccctacttcttgcatttggcctgctttgtttgccgtggttacaggagggctcggcacaagtgaccctgagagagtctggccccgctctggttaagcccacacagaccctgacactgacctgcaccttcagcggcttcagcctgagcacaagcggcatgtctgtcggctggatcagacagcctcctggcaaggctctggaatggctggccgacatttggtgggacgacaagaaggactacaaccccagcctgaagtccagactgaccatcagcaaggacaccagcaagaaccaggtggtgctgaaagtgaccaacatggaccctgccgacaccgccacctactactgcgccagatccatgatcaccaactggtacttcgacgtctggggcgccggcaccacagtgacagtttcttctagcaccaagggcccatcggtcttccccctggcaccctcctccaagagcacctctgggggcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggttaa

SEQ ID NO: 25 - DNA, 976 bp, synthetic construct
  misc_feature 1..976: note = V kappa 3-15 segment of the human IgK variable region and an HA tag
  source 1..976: mol_type = other DNA
ggcttgtgccaaacatcttctgggtggatttaggtgattgaggagaagaaagacacaggagcgaaattctctgagcacaagggaggagttctacactcagactgagccaacagacttttctggcctgacaaccagggcggcgcaggatgctcagtgcagagaggaagaaacaggtggtctctgcagctggaagctcagctcccaccccagctgctttgcatgtccctcccagctgccctaccttccagagcccatatcaatgcctgggtcagagctctggggaggaactgctcagttaggacccagacggaaccatggaagccccagcgcagcttctcttcctcctgctactctggctcccaggtgaggggaatatgaggtgtctttgcacatcagtgaaaactcctgccacctctgctcagcaagaaatataattaaaattcaatgtagatcaacaattttggctctacttaaagacagtgggtttgattttgattacatgagtgcatttctgttttatttccaatttcagataccactggagaaatagtgatgacgcagtctccagccaccctgtctgtgtctccaggggaaagagccaccctctcctgcacttacccctatgatgtgcctgactacgcttgcacttacccctatgatgtgcctgactacgcttgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcccacagtgattcaacatgaaacaaaaacctcaagaagaccatcagtgtttactagattataccagctgcttcctttacaga

SEQ ID NO: 26 - DNA, 36 bp, synthetic construct
  misc_feature 1..36: note = SA 97.66 splice acceptor
  source 1..36: mol_type = other DNA
ttcctttttttttcaggtgtcgtgaaaactacccct

SEQ ID NO: 27 - DNA, 575 bp, synthetic construct
  misc_feature 1..575: note = ECMV IRES
  source 1..575: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 28 - protein, 22 aa, synthetic construct
  REGION 1..22: note = P2A picornia Virus 2A peptide
GSGATNFSLLKQAGDVEENPGP

SEQ ID NO: 29 - DNA, 4683 bp, Adeno-associated virus
  misc_feature 1..4683: note = GenBank: AF028704.1; note = Adeno-associated virus 6, complete genome
  source 1..4683: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 30 - DNA, 4393 bp, Adeno-associated virus
  misc_feature 1..4393: note = NC_006261.1; note = Adeno-associated virus - 8, complete genome
  source 1..4393: mol_type = genomic DNA
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 31 - DNA, 41 bp, synthetic construct
  misc_feature 1..41: note = SA EF1a splice acceptor
  source 1..41: mol_type = other DNA
agtttttttcttccatttcaggtgtcgtgaaaactacccct

SEQ ID NO: 32 - DNA, 13 bp, synthetic construct
  misc_feature 1..13: note = SD mIGHJ1/J4 splice donor
  source 1..13: mol_type = other DNA
ctcctcaggtaag

SEQ ID NO: 33 - DNA, 23 bp, synthetic construct
  misc_feature 1..23: note = IgHJ3 SD 98.84 splice donor
  source 1..23: mol_type = other DNA
tgtctctgcaggtgagtcctaac

SEQ ID NO: 34 - DNA, 155 bp, synthetic construct
  misc_feature 1..155: note = Variant of IGH minimal promoter
  source 1..155: mol_type = other DNA
ggtaccttttcattccttcctctccagttcttctctagatggactaggtccttaactagcgaattcggatccctgtctcatgaatatgcaaatcaggtgagtccatggtggtaaatatagggatgtcgacacacctcacaaacttaagatctaga

SEQ ID NO: 35 - DNA, 314 bp, synthetic construct
  misc_feature 1..314: note = IgK Minimal Promoter
  source 1..314: mol_type = other DNA
ggcttgtgccaaacatcttctgggtggatttaggtgattgaggagaagaaagacacaggagcgaaattctctgagcacaagggaggagttctacactcagactgagccaacagacttttctggcctgacaaccagggcggcgcaggatgctcagtgcagagaggaagaagcaggtggtctttgcagctgaaagctcagctcccaccccagctgctttgcatgtccctcccagctgccctaccttccagagcccatatcaatgcctgggtcagagctctggggaggaactgctcagttaggacccagacggaacc

SEQ ID NO: 36 - DNA, 28 bp, synthetic construct
  misc_feature 1..28: note = 12-RSS
  misc_feature 8..19: note = n is a, c, g, or t
  source 1..28: mol_type = other DNA
cacagtgnnnnnnnnnnnnacaaaaacc

SEQ ID NO: 37 - DNA, 39 bp, synthetic construct
  misc_feature 1..39: note = 23-RSS
  misc_feature 8..30: note = n is a, c, g, or t
  source 1..39: mol_type = other DNA
cacagtgnnnnnnnnnnnnnnnnnnnnnnnacaaaaacc

SEQ ID NO: 38 - DNA, 38 bp, synthetic construct
  misc_feature 1..38: note = 22-RSS
  misc_feature 8..29: note = n is a, c, g, or t
  source 1..38: mol_type = other DNA
cacagtgnnnnnnnnnnnnnnnnnnnnnnacaaaaacc

SEQ ID NO: 39 - DNA, 27 bp, synthetic construct
  misc_feature 1..27: note = 11-RSS
  misc_feature 8..18: note = n is a, c, g, or t
  source 1..27: mol_type = other DNA
cacagtgnnnnnnnnnnnacaaaaacc

SEQ ID NO: 40 - DNA, 29 bp, synthetic construct
  misc_feature 1..29: note = 13-RSS
  misc_feature 8..20: note = n is a, c, g, or t
  source 1..29: mol_type = other DNA
cacagtgnnnnnnnnnnnnnacaaaaacc

SEQ ID NO: 41 - DNA, 40 bp, synthetic construct
  misc_feature 1..40: note = 24-RSS
  misc_feature 8..31: note = n is a, c, g, or t
  source 1..40: mol_type = other DNA
cacagtgnnnnnnnnnnnnnnnnnnnnnnnnacaaaaacc

SEQ ID NO: 42 - DNA, 108 bp, synthetic construct
  misc_feature 1..108: note = Line 1 of Fig 17A: IgK-V3-15 Sense strand, the Mouse IgK-J (any of J1, J2, J4, J5, that may be any sequence denoted herein as N for any nucleotide, in the middle block of line 1) and Mouse IgK-Constant Sense strand
  misc_feature 38..76: note = n is a, c, g, or t
  source 1..108: mol_type = other DNA
cagtttattactgtcagcagtataataactggcctccnnnnnnnnnnnnnnnnnnnnnnnnnnnnnnnnnnnnnnngggctgatgctgcaccaactgtatccatcttc

SEQ ID NO: 43 - DNA, 108 bp, synthetic construct
  misc_feature 1..108: note = Line 2 of Fig 17A: IgK-V3-15 anti-Sense strand, the Mouse IgK-J (any of J1, J2, J4, J5, that may be any sequence denoted herein as N for any nucleotide, in the middle block of line 1) and Mouse IgK-Constant Sense strand
  misc_feature 38..76: note = n is a, c, g, or t
  source 1..108: mol_type = other DNA
gtcaaataatgacagtcgtcatattattgaccggaggnnnnnnnnnnnnnnnnnnnnnnnnnnnnnnnnnnnnnnncccgactacgacgtggttgacataggtagaag

SEQ ID NO: 44 - protein, 27 aa, synthetic construct
  REGION 1..27: note = Line 3 of Fig 17A: putative encoded amino acid sequence of the IgK-V3-15, the Mouse IgK-J (any of J1, J2, J4, J5, that may be any sequence denoted herein as X for any amino acid residue, in the middle block of line 1) and Mouse IgK-Constant Sense strand
  REGION 14..26: note = MISC_FEATURE - x is any amino acid residue
AVYYCQQYNNWPPXXXXXXXXXXXXXG

SEQ ID NO: 45 - DNA, 35 bp, synthetic construct
  misc_feature 1..35: note = Line 4 of Fig 17A: IgK-V3-15 Sense strand at the left block of the figure
  source 1..35: mol_type = other DNA
cagtttattactgtcagcagtataataactggcct

SEQ ID NO: 46 - DNA, 32 bp, synthetic construct
  misc_feature 1..32: note = Line 4 of Fig 17A: Mouse IgK-Constant Sense strand at the right block of the figure. also the left part of lines 5 and 6
  source 1..32: mol_type = other DNA
gggctgatgctgcaccaactgtatccatcttc

SEQ ID NO: 47 - DNA, 34 bp, synthetic construct
  misc_feature 1..34: note = Line 5 of Fig 17A: Left part of coding joint #1
  source 1..34: mol_type = other DNA
cagtttattactgtcagcagtataataactggcc

SEQ ID NO: 48 - DNA, 38 bp, synthetic construct
  misc_feature 1..38: note = Line 5 of Fig 17A: Middle part of coding joint #1
  source 1..38: mol_type = other DNA
gctcacgttcggtgctgggaccaagctggagctgaaac

SEQ ID NO: 49 - DNA, 36 bp, synthetic construct
  misc_feature 1..36: note = Line 6 of Fig 17A: Left part of coding joint #2
  source 1..36: mol_type = other DNA
cagtttattactgtcagcagtataataactggcctc

SEQ ID NO: 50 - DNA, 36 bp, synthetic construct
  misc_feature 1..36: note = Line 6 of Fig 17A: Middle part of coding joint #2
  source 1..36: mol_type = other DNA
gcacgttcggctcggggacaaagttggaaataaaac

SEQ ID NO: 51 - DNA, 314 bp, synthetic construct; identical to SEQ ID NO: 35
  misc_feature 1..314: note = IgK "Minimal Promoter
  source 1..314: mol_type = other DNA

(19) United States
(12) Patent Application Publication
(10) Pub. No.: US 2024/0344088 A1
(43) Pub. Date: Oct. 17, 2024

(54) RECOMBINATION ACTIVATING GENE (RAG) INDUCED V(D)J GENE TARGETING

(71) Applicant: RAMOT AT TEL-AVIV UNIVERSITY LTD., TEL AVIV (IL)

(72) Inventors: Adi BARZEL, Hod Hasharon (IL); Iris DOTAN, Tel-Aviv (IL); Carmel PUNDAK-MINTZ, Tel-Aviv (IL); Daniel NATAF, Tel-Aviv (IL); Miriam FRIED, Tel-Aviv (IL); Natalia GRITSENKO, Tel-Aviv (IL)

(21) Appl. No.: 18/750,678

(22) Filed: Jun. 21, 2024

Related U.S. Application Data
(63) Continuation of application No. 16/606,240, filed on Oct. 18, 2019, now Pat. No. 12,060,572.
(60) Provisional application No. 62/487,799, filed on Apr. 20, 2017.

(51) Int. Cl. C12N 15/90; C07K 16/28; C12N 15/86

(52) U.S. Cl. CPC C12N 15/907; C07K 16/2809; C12N 15/86; C07K 2317/14; C07K 2317/24; C07K 2317/76; C12N 2510/00

(57) ABSTRACT

The present invention relates to methods for targeted insertion of at least one nucleic acid sequence/s of interest into a target genomic locus of a mammalian cell. More specifically, the methods of the invention are based on using nucleic acid cassettes comprising the nucleic acid sequence/s of interest and at least one recognition signal sequence (RSS), for insertion of the nucleic acid sequence of interest into the target genomic locus that is mediated by RAG-catalyzed recombination. The invention further provides cassettes, vectors and vehicles and cells comprising said cassettes, compositions and uses thereof in immunotherapy.

The Sequence Listing XML of 101,871 bytes in size, created on Jun. 21, 2024, with the file name “2024 Jun. 21 SeqLst-BARZEL9A” filed in the U.S. Patent and Trademark even date herewith), is hereby incorporated herein by reference.

### FIELD OF THE INVENTION

The invention relates to immunotherapy. More specifically, the invention relates to methods and compositions for engineering T cells and B cells using V(D)J recombination, compositions, methods and uses thereof in immunotherapy.

### BACKGROUND ART

References considered to be relevant as background to the presently disclosed subject matter are listed below:
[1] Beatty G L, Haas A R, Maus M V, et al. Mesothelin-specific chimeric antigen receptor mRNA-engineered T cells induce anti-tumor activity in solid malignancies. Cancer Immunol Res. 2014; 2 (2): 112-120.
[2] Liu X, Ranganathan R, Jiang S, et al. A Chimeric Switch-Receptor Targeting PD1 Augments the Efficacy of Second-Generation CAR T Cells in Advanced Solid Tumors. Cancer Res. 2016; 76 (6): 1578-1590.
[3] Sharpe M, Mount N. Genetically modified T cells in cancer therapy: opportunities and challenges. Dis Model Mech. 2015; 8 (4): 337-350.
[4] Poirot L, Philip B, Schiffer-Mannioui C, et al. Multiplex Genome-Edited T-cell Manufacturing Platform for “Off-the-Shelf” Adoptive T-cell Immunotherapies. Cancer Res. 2015; 75 (18): 3853-3864.
[5] Garfall A L, Maus M V, Hwang W T, et al. Chimeric Antigen Receptor T Cells against CD19 for Multiple Myeloma. N Engl J Med. 2015; 373 (11): 1040-1047.
[6] Maude S L, Frey N, Shaw P A, et al. Chimeric antigen receptor T cells for sustained remissions in leukemia. N Engl J Med. 2014; 371 (16): 1507-1517.
[7] Zhao Y, Parkhurst M R, Zheng Z, et al. Extrathymic generation of tumor-specific T cells from genetically engineered human hematopoietic stem cells via Notch signaling. Cancer Res. 2007; 67 (6): 2425-2429.
[8] Themeli M, Kloss C C, Ciriello G, et al. Generation of tumor-targeted human T lymphocytes from induced pluripotent stem cells for cancer therapy. Nat Biotechnol. 2013; 31 (10): 928-933.
[9] Hacein-Bey-Abina S, Garrigue A, Wang G P, et al. Insertional oncogenesis in 4 patients after retrovirus-mediated gene therapy of SCID-X1. J Clin Invest. 2008; 118 (9): 3132-3142.
[10] Bryant L M, Christopher D M, Giles A R, et al. Lessons learned from the clinical development and market authorization of Glybera. Hum Gene Ther Clin Dev. 2013; 24 (2): 55-64
[11] Barzel A, Paulk N K, Shi Y, et al. Promoterless gene targeting without nucleases ameliorates haemophilia B in mice. Nature. 2015; 517 (7534): 360-364.
[12] Sather B D, Romano Ibarra G S, Sommer K, et al. Efficient modification of CCR5 in primary human hematopoietic cells using a megaTAL nuclease and AAV donor template. Sci Transl Med. 2015; 7 (307): 307ra156.
[13] Boutros C, Tarhini A, Routier E, et al. Safety profiles of anti-CTLA-4 and anti-PD-1 antibodies alone and in combination. Nat Rev Clin Oncol. 2016; 13 (8): 473-486.
[14] Luo X M, Maarschalk E, O'Connell R M, Wang P, Yang L, Baltimore D. Engineering human hematopoietic stem/progenitor cells to produce a broadly neutralizing anti-HIV antibody
[15] Fusil F, Calattini S, Amirache F, et al. A Lentiviral Vector Allowing Physiologically Regulated Membrane-anchored and Secreted Antibody Expression Depending on B-cell Maturation Status. Mol Ther. 2015; 23 (11): 1734-1747
[16] Moreau A, Vicente R, Dubreil L, et al. Efficient intrathymic gene transfer following in situ administration of a rAAV serotype 8 vector in mice and nonhuman primates. Mol Ther. 2009; 17 (3): 472-479.
[17] Richter M, Saydaminova K, Yumul R, et al. In vivo transduction of primitive hematopoietic stem cells after mobilization and intravenous injection of integrating adenovirus vectors. Blood. 2016.
[18] Chen Z, Fischer E R, Kouiavskaia D, et al. Cross-neutralizing human anti-poliovirus antibodies bind the recognition site for cellular receptor. Proc Natl Acad Sci USA. 2013; 110 (50): 20242-20247.
[19] Schlereth B, Kleindienst P, Fichtner I, et al. Potent inhibition of local and disseminated tumor growth in immunocompetent mouse models by a bispecific antibody construct specific for Murine CD3. Cancer Immunol Immunother. 2006; 55 (7): 785-796.

Acknowledgement of the above references herein is not to be inferred as meaning that these are in any way relevant to the patentability of the presently disclosed subject matter.

### BACKGROUND OF THE INVENTION

Cancer immunotherapy harnesses and augments immune mechanisms to fight malignancies. In particular, adoptive T cell transfer entails the activation and expansion of T cells that target tumor associated antigens (TAA). Naturally occurring T cell receptors (TCRs) often have low affinity against TAAs. However, T cells can be engineered to express highly potent TCRs, which can recognize fragments of both intracellular and cell surface proteins when presented in a major histocompatibility complex (MHC) context. Alternatively, T cells can be engineered to express chimeric antigen receptors (CARs), which have a high TAA affinity. CARs recognize intact cell surface proteins in an MHC independent manner, and are thus insensitive to cancer escape mechanisms associated with MHC loss. Second and third generation CARs have an increased potency due to inclusion of multiple co-stimulatory domains, allowing highly promising clinical results for several hematological malignancies. Treatment of solid tumors may in turn be facilitated by identifying and targeting cancer specific antigens, by using short-lived CAR mRNA to reduce toxicity [1] and by counteracting inhibitory immune checkpoints [2]. However, large scale application of adoptive T cell therapy may be extremely challenging. State of the art T cell engineering for cancer immunotherapy relies on ex vivo manipulations. It is conducted in a limited number of specialized medical centers, because it requires dedicated facilities and expertise in clinical grade collection, purification and activation of T cell products, as well as in viral vector manipulations [3]. Furthermore, autologous T cell engineering and expansion is time-consuming, delaying treatment of patients in need. These challenges can be partially mitigated by the recent development of universal allogenic CAR T cells (UCART) which underwent nuclease-mediated knockout of the endogenous TCR to prevent graft vs. host disease (GVHD) as well as knockout of the CD52 gene, allowing expansion in the presence of the conditioning drug Alemtuzumab [4]. However, risk remains of GVHD from residual TCR coding T cells, and even “off-the-shelf” cell products may require complex cryopreservation and subsequent expansion.

Treatment outcome is determined by the subset composition of the targeted T cell population. Both CD4 and CD8 CAR modified T cells participate in an effective cancer immunotherapy. In particular, naïve and central memory effector T cells are most effective in inducing remission. However, in trials reported to date, the modified T cell population often included also terminally differentiated and exhausted T cells that contribute little to the treatment efficacy [5-6]. Therefore, methods have been developed for the introduction of CAR and engineered TCRs into pluripotent cells ((Hematopoietic stem cells (HSCs) and Induced Pluripotent Stem Cells (iPSCs)) which are later differentiated into T cells [7-8]. However, in pluripotent cells, the promiscuously integrating vectors used to date, including retroviral and lentiviral vectors as well as transposons, may induce clonal expansion and cancer by integrating near oncogenes leading to their aberrant expression [9]. In contrast, the Recombinant adeno-associated virus (rAAV) is a safe and potent vector that is widely used in gene therapy trials and approved drugs [10]. The AAV virus is non-pathogenic and the rAAV vector encodes no viral genes. rAAV is less prone to insertional mutagenesis, is not associated with adverse immunogenicity, can be produced in high titers, and requires no toxic pseudotyping for in vivo applications. Importantly, several rAAV serotypes can efficiently transduce HSCs and lymphocytes. However, rAAV is not commonly used for Immunotherapy because it rarely integrates to allow stable expression from dividing cells.

The inventors recently demonstrate that rAAV can be used to facilitate stable expression at therapeutic levels from a dividing tissue following site-specific gene integration into the genome [11]. In particular, rAAV was used to facilitate nuclease-independent insertion of a promoterless therapeutic transgene to be expressed only upon integration by homologous recombination (HR) into the liver-specific highly-expressed albumin locus. Using this method, sustained amelioration of genetic diseases such as haemophilia B38, Crigler Najjar syndrome, and methylmalonic academia, was demonstrated after a single systemic injection to either neonatal or adult mice. Other groups have attempted site specific integration of CAR genes into T cells [12]. Basal rates of HR in T cells and HSC are low, but can be increased by several orders of magnitude by the introduction of targeted DNA breaks using site-specific nucleases. However, these nucleases have off-target effects that are difficult to monitor. Even on target, a double strand break, induced by a nuclease, leads more often to mutagenic non-homologous end joining (NHEJ) than to the desired gene insertion by HR. The nucleases also include microbial protein domains that, if used in vivo, may be immunogenic, leading to elimination of targeted cells, or even worse, to an anaphylactic response. An optimal lymphocyte engineering technology should allow locus specific integration of the receptor and/or Antibody gene without relying on bacterial nucleases.

Monoclonal Antibodies are used widely to treat cancer, autoimmune diseases, and more. Mouse Antibodies, used in early day therapies, were often immunogenic and have been replaced with humanized Antibodies and fully human Antibodies produced using transgenic animals or phage display. In addition, different Antibody fragments and modified Antibodies are finding increasing utility, including single chain variable fragments (scFv), bi-specific and tri-specific Antibodies. Recent years have seen a great surge in the application of monoclonal Antibodies that block inhibitory immune checkpoints, and thus allow the immune system to better fight the malignancy. Examples include anti-PD1 and anti CTLA-4 Antibodies which have already transformed the management of patients with advanced-stage melanoma and may be highly efficient in the treatment of many other cancers [13]. Nevertheless, for most indications, monoclonal Antibody therapy is rarely curative. An Antibody has a limited half-life and has to be repeatedly administered at a very high financial cost and with aggravated risk for the development of anti-drug antibodies. B cells engineering may enable safely expression of desired antibodies and thus may represent a potent and sustained immunotherapy activated by an antigen and naturally augmented by affinity maturation. However, B cell engineering has received little attention due to concerns about sustained autoimmunity and challenges in activation and avoidance of tolerance. Luo et al. have engineered human hematopoietic stem/progenitor cells to produce an anti-HIV Antibody upon in vitro maturation to B cells [14]. However, expression levels were constitutively low as the lentivector-coded Antibody did not allow B cell activation. Fusil et al., used a different lentivector design that allowed B cell activation and differentiation into Antibody producing plasma cells [15]. However, the promiscuous and ectopic lentiviral integration as well as the synthetic polyadenylation sites that were used prevented native regulation on the transition from BCR expression to antibody secretion and did not allow for affinity maturation upon activation. In addition, lentiviral transduction is ill-fitted for in vivo applications, thus subjecting B cell engineering to similar challenges to those discussed above with regards to T cell engineering.

There is therefore a crucial unmet need to design scalable immunotherapies to treat the very large population of patients with cancer and autoimmune diseases.

The present invention address these issues by providing a revolutionary technology, named “VDJ targeting”, for the engineering of B cells as well as T cells, both in vivo and ex vivo.

### SUMMARY OF THE INVENTION

In a first aspect, the invention relates to a method for targeted insertion of at least one nucleic acid sequence of interest into a target genomic locus of a mammalian cell. More specifically, the method of the invention comprises the step of contacting a mammalian cell expressing the recombination activating gene (RAG) complex with at least one nucleic acid cassette comprising the nucleic acid sequence/s of interest and at least one recognition signal sequence (RSS), or with a vector comprising the cassette. It should be noted that the insertion of the nucleic acid sequence of interest into the target genomic locus is facilitated, mediated and performed by RAG-catalyzed recombination. More specifically, such recombination may occur in some embodiments between at least one genomic RSS flanking the target genomic locus and at least one RSS comprised within the nucleic acid cassette.

In a second aspect, the invention relates to a method for targeted insertion of at least one nucleic acid sequence of interest into a target genomic locus of a cell in a mammalian subject. More specifically, the method may comprise the step of administering to the subject an effective amount of at least one nucleic acid cassette comprising the nucleic acid sequence of interest and at least one RSS. The invention further encompasses in some embodiments thereof the administration of a vector comprising the cassette of the invention or any composition thereof to the subject. It should be noted that the nucleic acid cassette administered by the method of the invention enables the insertion of a nucleic acid sequence of interest into a target genomic locus.

A further aspect of the invention relates to a method of treating, preventing, ameliorating, inhibiting or delaying the onset of a pathologic disorder in a mammalian subject. More specifically, the method of the invention may comprise the step of administering to the treated subject an effective amount of at least one of: (a) nucleic acid cassette; (b) a vector, vehicle or composition comprising said nucleic acid cassette; and (c) a cell transduced or transfected with the nucleic acid cassette or with a vector comprising said cassette, or any combinations or compositions thereof. It should be noted that the cassette administered by the method of the invention may comprise at least one nucleic acid sequence of interest and at least one RSS. In yet some further embodiments, the cassette used by the method of the invention may lead to targeted insertion of at least one nucleic acid sequence of interest into a target genomic locus in at least one cell of the treated subject. The insertion of the nucleic acid sequence of interest into the target genomic locus in cell/s of the treated subject may be facilitated, mediated and/or performed by RAG-catalyzed recombination between at least one genomic RSS flanking the target genomic locus and at least one RSS comprised within the nucleic acid cassette.

In yet a further aspect, the invention relates to a nucleic acid cassette. More specifically, the cassette of the invention may comprise at least one nucleic acid sequence of interest and at least one RSS. It should be further noted that in some embodiments, the cassette provided by the invention may be suitable for targeted insertion of at least one nucleic acid sequence of interest into a target genomic locus in a mammalian cell. In more specific embodiments, the insertion of the nucleic acid sequence of interest into the target genomic locus may be facilitated, mediated and/or performed by RAG-catalyzed recombination between at least one genomic RSS flanking the target genomic locus and at least one RSS comprised within the nucleic acid cassette.

In yet a further aspect, the invention relates to a pharmaceutical composition comprising at least one nucleic acid cassette, or any vector or cell comprising said cassette. More specifically, the cassette of the composition of the invention may comprise the nucleic acid sequence of interest and at least one RSS. In yet some further embodiments, the cassette provided by the composition of the invention may be used for targeted insertion of at least one nucleic acid sequence of interest into a target genomic locus. In more specific embodiments, the compositions of the invention may comprise any of the nucleic acid cassettes of the invention. In some optional embodiments, the compositions of the invention may further comprise at least one of pharmaceutically acceptable carrier/s, diluent/s, excipient/s and additive/s.

Further aspects of the invention concern a therapeutically effective amount of the nucleic acid cassette in accordance with the invention and any compositions, vectors or vehicles thereof for use in a method for targeted insertion of at least one nucleic acid sequence of interest into a target genomic locus of a mammalian cell. In some embodiments, the cassettes for use in accordance with the invention may comprise at least one nucleic acid sequence of interest and at least one RSS. In more specific embodiments, the insertion of the nucleic acid sequence of interest into the target genomic locus is mediated by RAG-catalyzed recombination between at least one genomic RSS flanking the target genomic locus and at least one RSS comprised within said nucleic acid cassette. In yet some further aspects, the invention provides a therapeutically effective amount of the nucleic acid cassette in accordance with the invention and any compositions, vectors, vehicles or cells comprising said cassette, for use in a method for treating, preventing, ameliorating, inhibiting or delaying the onset of a pathologic disorder in a mammalian subject. In some embodiments, the cassettes for use in accordance with the invention may comprise at least one nucleic acid sequence of interest and at least one RSS. Thus, in some embodiments these cassettes lead to targeted insertion of at least one nucleic acid sequence of interest into a target genomic locus in the treated subject. In more specific embodiments, the insertion of the nucleic acid sequence of interest into the target genomic locus is mediated by RAG-catalyzed recombination between at least one genomic RSS flanking the target genomic locus and at least one RSS comprised within said nucleic acid cassette.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. AAV vector

Schematic representation of the construct incorporated into the double strand AAV vector.

FIG. 2. dsAAV 360-A vector

Schematic map of the dsAAV 360-A vector. More specifically, the figure presents a map of a bacterial plasmid used in the preparation of the double stranded AAV vector (dsAAV) also referred to herein as self complementary AAV vector (ScAAV).

FIG. 3. VDJ targeting

Schematic representation of the location of the primers used for the Reverse transcription reactions analyzing the recombination products.

FIG. 4A-4B. V(D)J recombination and VDJ targeting

FIG. 4A shows natural V(D)J recombination during lymphocyte development. D to J recombination precedes V to DJ recombination

FIG. 4B shows VDJ targeting: The rAAV vector encodes a receptor or Ab gene flanked by RSSs. Transduction of lymphocyte precursors, which express RAG, facilitates insertion of the receptor/Ab gene between gene segments. The J recombination event may precede the V recombination event. The receptor/Ab gene is preceded by a sequence coding for a 2A peptide or an IRES to prevent protein fusions to preceding segments. A splice donor (SD) may be provided to allow use of the endogenous C segments, upon integration and expression.

FIG. 5A-5B. RSS configurations

FIG. 5A shows specific RSS configurations and resulting VDJ targeting in the B lineage.

FIG. 5B shows specific RSS configurations and resulting VDJ targeting in the T lineage.

Triangles: RSSs, Rectangles: coding regions and transgene cassettes, Arrows: RAG recombination, SD (splice donor), Ab (antibody), CAR (chimeric antigen receptor), TCR (T cell receptor).

FIG. 6A-6B. Reporter systems

FIG. 6A illustrates an example of reporter system for RAG activity.

FIG. 6B. illustrates an example of reporter system for VDJ targeting in cell lines.

The reporter may be either episomal or integrated. Different donor and reporter constructs have different RSS sequences at different relative orientation in order to assess compatibility and optimize efficiency.

FIG. 7A-7B. VDJ targeting of a single-RSS construct to the IgK locus of HEK293T cells

HEK293T cells were transfected with the dsRED2-Con23 plasmid containing a 23-consensus RSS and with plasmids encoding RAG1 and RAG2

FIG. 7A provides a Scheme of VDJ targeting between the IgK locus and the dsRED2-Con23 plasmid. Arrows with numbering 1 and 2 indicate the position of forward and reverse primers for subsequent PCR analysis.

FIG. 7B shows PCR product of recombination between the IgK locus and the dsRED2-Con23 plasmid, using primers forward/reverse designated by arrows 1 and 2 (on FIG. 7A).

FIG. 8A-8B. VDJ targeting of a dual-RSS construct to the IgK locus of HEK293T cells HEK293T cells were transfected with the GFPi-redless plasmid containing a 23-consensus RSS and a 12-consensus RSS and with plasmids encoding RAG1 and RAG2.

FIG. 8A provides a scheme of VDJ targeting between the IgK locus and the GFPi plasmid. Arrows with numbering 1 and 3 indicate the position of forward and reverse primers for subsequent PCR analysis.

FIG. 8B shows PCR product of recombination between the IgK locus and the GFPi plasmid, using primers designated by arrows 1 and 3 (on FIG. 8A).

FIG. 9A-9B. VDJ targeting of a double-RSS construct to the IgK locus of inducibly differentiating B cells

V-Abl immortalized murine Pro B cells were transfected with the GFPi-redless plasmid containing a 23-consensus RSS and a 12-consensus RSS and differentiation was induced using Imatinib.

FIG. 9A provides a scheme of VDJ targeting between the IgK locus and the GFPi-Redless plasmid. Arrows with numbering 1 and 3 indicate the position of forward and reverse primers for subsequent PCR analysis.

FIG. 9B shows PCR product of recombination between the IgK locus and GFPi-redless plasmid, using primers designated by arrows 1 and 3 (on FIG. 9A).

FIG. 10. pADN110 plasmid

Schematic map of the donor DNA i.e. the pADN110 plasmid comprising the nucleic acid sequence of interest (e.g., Palivizumab coding sequences).

FIG. 11. VDJ targeting of antibody in inducibly differentiating B cells

DNA agarose gel of RT-PCR analysis confirming integration of the gene coding for the anti-RSV Palivizumab antibody into the IgK locus of B cell progenitors only in the presence of Imatinib.

FIG. 12. VDJ recombination in B cell progenitors

Diagram representing B-cell lineage and the period of IL7-receptor enrichment.

FIG. 13A-13C. IL7R enrichment

Flow cytometry profiles of murine bone marrow cells labeled with anti IL7R-PE and magnetic nano-beads, subjected to the enrichment protocol.

FIG. 13A (i-iv) are flow cytometry profiles of total loaded material.

FIG. 13B (i-iv) are flow cytometry profiles of column flow through.

FIG. 13C (i-iv) are flow cytometry profiles of column eluted IL7R-PE labeled cells.

FIG. 14. CD19 staining of IL7R enriched cells

Bivariate plots of the IL7R positive gated cells. Cells at time point 0 after IL7R enrichment were washed and stained for CD19.

FIG. 15A-15B. Differentiation in IL7R enriched cells

Flow cytometry analysis showing differentiation of cultured IL7R positive cells.

FIG. 15A. Bivariate plots of cells at time point 0 (isolation) stained for IgK and IgM.

FIG. 15B. Bivariate plots of cells following 3 days of culture, stained for IgK and IgM.

FIG. 16A-16C. RT analysis of IL7R+ enriched cells transduced with dsAAV 360-A

FIG. 16A. Schematic representations of the expected trans-recombination products. The black arrows indicate the position of the primers.

FIG. 16B. shows PCR product of the trans-recombination product using primers designated by arrows on FIG. 16A.

FIG. 16C. DNA agarose gels showing bands corresponding to the putative expected size of the trans-recombination product (marked by asterisk).

FIG. 17. Coding joint diversity

FIG. 17A. Schematic illustration of the coding joint junctional diversity—a unique attribute of RAG mediated integration, comprising the 5′ to 3′ fragment of the coding joints IgK-J4 and IgK-J5. More specifically, Line 1 of the figure shows IgK-V3-15 Sense strand, the Mouse IgK-J (any of J1, J2, J4, J5, that may be any sequence denoted herein as N for any nucleotide, in the middle block of line 1) and Mouse IgK-Constant Sense strand, as denoted by SEQ ID NO 42, Line 2 of the figure shows IgK-V3-15 anti-Sense strand, the Mouse IgK-J (any of J1, J2, J4, J5, that may be any sequence denoted herein as N for any nucleotide, in the middle block of line 1) and Mouse IgK-Constant Sense strand, as denoted by SEQ ID NO. 43. Line 3 of the figure shows putative encoded amino acid sequence of the IgK-V3-15, the Mouse IgK-J (any of J1, J2, J4, J5, that may be any sequence denoted herein as X for any amino acid residue, in the middle block of line 1) and Mouse IgK-Constant Sense strand, as denoted by SEQ ID NO. 44. Line 4 of the figure shows IgK-V3-15 Sense strand at the left block of the figure, as denoted by SEQ ID NO 45. The right block of line 4 of the figure shows Mouse IgK-Constant Sense strand. The same sequence is also shown at the left block of lines 5 and 6, as denoted by SEQ ID NO 46. Line 5 of the figure shows Left part of coding joint #1, as denoted by SEQ ID NO 47. The Middle part of line 5 of the figure shows Middle part of coding joint #1, as denoted by SEQ ID NO 48. Line 6 of the figure shows the Left part of coding joint #2, as denoted by SEQ ID NO. 49. The Middle part of line 6 shows Middle part of coding joint #2, as denoted by SEQ ID NO 50.

FIG. 17B. Schematic illustration of the 2 coding joints map to IgK-J4 and IgK-J5.

FIG. 18. Intrathymic injection

Fluorescent signals obtained from thymuses extracted postmortem from mice after live intrathymic injections of CFSE stained Jurkat cells.

FIG. 18A. is a photograph from a first mouse after extraction while FIG. 18B and FIG. 18C are photographs from a second mouse before and after extraction respectively.

FIG. 19. Intrathymic injection of Luciferase expressing cells

Live imaging photographs of mice after intrathymic injection of Luciferase expressing cells.

FIG. 19A is a photograph of a mouse injected with fresh bone marrow cells transduced with a luciferase expressing retroviral vector.

FIG. 19B is a photograph of a mouse injected with Lin-enriched bone marrow cells transduced with a luciferase expressing retroviral vector.

FIG. 19C is a photograph of a control, non-injected mouse.

FIG. 20. Targeting B cells for inducible secretion of an autoantibody

Two independent cassettes are separately targeted: one coding for a secreted Antibody and one coding for a BCR targeting different antigens. Each gene is preceded by an IRES to allow separate translation. The secreted Antibody is preferably a scFv or a BiTE. The Antibody is directed against a disease associated autoantigen, and the BCR is directed against a foreign inducer. The B cell will differentiate to Antibody-secreting plasma cells only upon administration of the foreign inducer.

### DETAILED DESCRIPTION OF THE INVENTION

T cell engineering for immunotherapy has shown clinical success, but large scale application is hindered by reliance on cumbersome ex vivo manipulations, performed in specialized centers only. In addition, B cell engineering has not shown therapeutic efficacy to date. Recombinant adeno associated vectors (rAAV) allow in vivo lymphocyte transduction but have seldom been used for lymphocyte engineering because they rarely integrate for stable expression in dividing cells. The present invention discloses herein a novel immunotherapy approach targeting immune genes into the genome using V(D)J recombination in developing lymphocytes. In some specific and non-limiting embodiments these immune-genes of interest may be delivered using any delivery vehicle, for example, a viral vehicle such as an adeno-associated virus (AAV), specifically, a recombinant AAV, (rAAV). The invention provides methods and compositions for ex vivo targeting of differentiating lymphocytes, demonstrating the feasibility and applicability thereof in the treatment and prophylaxis of pathologic disorders, specifically immune-related disorders such as cancer, autoimmune disease, pathogenic infections and immunodeficiencies (e.g., primary as well as secondary). Specifically, the methods, compositions and cassettes of the invention allow specific targeting of T cell receptors (TCRs) or chimeric antigen receptors (CARs) genes into loci coding TCR chains and specific targeting of antibody and B cell receptor genes into loci coding Immunoglobulin chains. The targeted antibodies or receptors are expressed under the endogenous regulation, only from the right cells at the right time and only at the correct amount.

More specifically, VDJ targeting in accordance with the invention, relies on the ability of the RAG complex to act in trans, i.e. to recombine RSSs found on different molecules. Indeed, by its evolution, structure and mechanism of action, RAG is a DNA transposase inducing the transposition of DNA segments in trans both in vitro and in vivo. While the efficient trans activity of RAG clearly speaks for the feasibility of VDJ targeting it is important to note that VDJ targeting is not a transposition event. In VDJ targeting cassettes provided by the invention, the relative orientation of the two RSSs precludes transposition but instead integration requires recombination of each vector RSS with an appropriate genomic RSS, thereby providing a targeted accurate insertion of the nucleic acid sequence of interest into the desired locus. Importantly, RAG has been shown to readily act in trans not only for transposition but also when the two RSSs are on different molecules. Unlike transposition, VDJ targeting is highly specific and as a second safety measure, the inventors refrain from overexpressing RAG, but rather rely on its endogenous expression only. In some embodiments, VDJ targeting as provided by the invention, may further rely on any delivery vehicle, specifically, a viral vector and more specifically, rAAV transduction. rAAV is proficient in transducing many different tissues in vivo, several rAAV variants can efficiently transduce HSCs and lymphocytes. In particular, rAAV is efficient, and superior to lentivectors, at in vivo intrathymic transfection in both mice and macaques.

VDJ targeting as provided by the methods, cassettes, compositions and uses described by the present invention, offers many advantages over state of the art technologies in safety, efficacy and scalability.

More specifically, state of the art T cell engineering is performed ex vivo using retrovectors, lentivectors or transposons, which are promiscuously integrating and bearing the risk of insertional mutagenesis and oncogene activation, particularly when applied to HSCs. In vivo use is not only genotoxic but may also requires toxic pseudotyping. In contrast, rAAV vectors are less genotoxic and are used in vivo without pseudotyping in multiple clinical trial trials and a EU approved drug. AAV is Non-pathogenic, and moreover, rAAV has no viral genes or promoters. Integration of a nucleic acid sequence of interest, is locus-directed and expression is from desired cell type only, while the promoterless vector diminishes risk of oncogene activation even upon rare off-target integration. State of the art nucleases can mediate receptor/antibody (Ab) targeting but they bear genotoxicity risk from both on and off-target mutations, as well as an immunogenicity risk if applied in vivo. In contrast, VDJ targeting in accordance with the invention, is independent of exogenous nucleases. VDJ targeting does not contribute to RAG associated genomic instability because RAG is not over-expressed, but instead targeting relies on natural RAG expression only, and the RSS orientation on the vector precludes transposition into the genome. Lymphocytes, engineered by state of the art technologies, may cross-react with autoantigens leading to adverse immune response. In contrast, in VDJ targeting in accordance with the invention, developing lymphocytes are targeted which are subjected to negative selection, thus diminishing the risk of undesired reactivity. With state of the art technologies, the chains of a targeted receptor may dimerize with those of the endogenous receptor leading to potentially dangerous undesired reactivity. In contrast, VDJ targeting at the endogenous locus as provided by the present invention, induces allelic exclusion and ensures expression of the desired receptor only.

Still further, state of the art vectors used in immunotherapy, including retrovectors, lentivectors and transposons do not allow high rates of in vivo transduction. In contrast, rAAV as used in some specific and non-limiting embodiments by the methods and compositions of the invention, is proficient in transducing many different tissues in vivo, and several rAAV variants can efficiently transduce HSCs and lymphocytes. In particular, rAAV8 is superior to lentivectors at in vivo intrathymic transfection in mice and in macaques. With state of the art technologies multiple lymphocyte subtypes are transduced, including exhausted cells. In contrast, in VDJ targeting as provided by the invention, only developing lymphocytes are engineered, leading to expression of the receptor/Ab gene in potent naïve cells. With state of the art technologies the targeted receptor may compete with the endogenous receptor and heterodimers on membrane presentation and cell activation. In contrast, VDJ targeting in accordance with the invention, induces allelic exclusion so that only the targeted receptor/Ab is expressed. Most importantly, state of the art technology does not allow B cell engineering for well controlled antibody secretion. In contrast, with VDJ targeting as disclosed by the invention, an Antibody gene can be inserted at an endogenous Antibody chain locus of a B cell in order to express a BCR and, upon antigen-induced activation, express a secreted antibody subjected to affinity maturation, including somatic hypermutation and class switching, for a potent immune response with reduced risk for antigenic escape and with memory retention.

Moreover, state of the art immunotherapy relies on cumbersome, expensive and time consuming ex vivo manipulations that can be performed in specialized centers only. In contrast, the VDJ targeting methods and compositions of the invention provide potent immunotherapeutic tool that may be applied safely to a much larger population of patients with diverse indications, in community clinics rather than specialized centers, at a lower cost and at a shorter time frame than possible with state of the art technologies.

VDJ targeting as provided by the present invention revolutionizes immunotherapy as it allows safe, efficient and scalable engineering of B cells and T cells, both in vivo and ex vivo. In addition, VDJ targeting has many important applications in basic science, biotechnology and medicine outside of immunotherapy alone.

In basic immunology studies, the technology can be used to target different reporters and allow studying the control over the expression of the endogenous locus. In addition, VDJ targeting can be used to study mechanistic aspects of the natural V(D)J recombination process, including parameters relating to the RSS sequences, RAG activity and differential gene segment availability. VDJ targeting could be used to target a barcoded library of receptors/Abs and follow cell population dynamics during development and subsequent to activation. Targeting of barcoded libraries can further be used in screens searching for optimal targeting of specific antigens. The output receptor/Ab of the screen could later be used in cell engineering for immunotherapy or in monoclonal Ab therapy. Finally, non-immune genes may be integrated into lymphocytes by VDJ targeting, and the product subsequently secreted, as a safe approach to augment the plasma levels of a desired protein.

Thus, in a first aspect, the invention relates to a method for targeted insertion of at least one nucleic acid sequence of interest into a target genomic locus of a mammalian cell. More specifically, the method of the invention may comprise the step of contacting a mammalian cell expressing the recombination activating gene (RAG) complex with at least one nucleic acid cassette comprising the nucleic acid sequence/s of interest and at least one recognition signal sequence (RSS), or with a vector comprising the cassette. It should be noted that the insertion of the nucleic acid sequence of interest into the target genomic locus is facilitated, mediated and/or performed by RAG-catalyzed recombination between at least one genomic RSS flanking the target genomic locus and at least one RSS comprised within the nucleic acid cassette.

The present invention therefore provides safe, specific and efficient methods for V(D)J targeted insertion of a nucleic acid sequence of interest into a target genomic locus. This specific insertion is based on RAG complex expressed in the target cells, that catalyze the specific recombination. By “recombination” it is meant a process of exchange of genetic information between two polynucleotides. RAG catalyzed recombination as used herein, refers to recombination process performed by the RAG complex, for example, during V(D)J recombination. More specifically, the V(D)J recombination process cleaves and splices variable (V), diversity (D), and joining (J) non-contiguous immunoglobulin (Ig) segments in the genome. Ig heavy chains and T cell receptor (TCR) β chains are formed by sequential steps of D-J and V-DJ recombination, while Ig light chains and TCR α chains are generated by direct VJ recombination. The critical cleavage step in V(D)J recombination is executed by the lymphocyte-specific enzyme containing the multi-domain proteins recombination-activating gene 1 and 2 (RAG1 and RAG2). These two recombination-activating gene products, whose cellular expression is restricted to lymphocytes during their developmental stages, are essential to the generation of mature B and T lymphocytes.

RAG recognizes specific recombination signal sequences (RSSs) flanking the 3′ end of the V, D, and J segments. RSSs are in some embodiments, genetic elements composed of a conserved heptamer (consensus: 5′CACAGTG-3′), a poorly conserved spacer of either 12±1 or 23±1 bp, and a conserved nonamer (consensus: 5′-ACAAAAACC-3′) as revealed by sequence alignments of RSSs. These RSSs are designated as 12-RSS or 23-RSS after the length of the spacer. Thus, in some specific embodiments RSS applicable in the present invention may comprise a nucleic acid sequence that includes the heptamer, followed by a 12 nucleotide spacer (N may be any nucleotide), and the conserved nonamer, specifically, 5′-CACAGTG NNNNNNNNNNNN ACAAAAACC, as denoted by SEQ ID NO. 36. In yet another option, the RSS applicable in the present invention may comprise a nucleic acid sequence that includes the heptamer, followed by a 23 nucleotide spacer (N may be any nucleotide), and the conserved nonamer, specifically, 5′-CACAGTG NNNNNNNNNNNNNNNNNNNNNNN ACAAAAACC, as denoted by SEQ ID NO. 37. In yet another option, the RSS applicable in the present invention may comprise a nucleic acid sequence that includes the heptamer, followed by a 22 nucleotide spacer, and the conserved nonamer, specifically, 5′-s, as denoted by SEQ ID NO. 38. Still further, in some embodiments, the spacer may range between 12±1 or 23±1 bp, and therefore, RSSs applicable in the present invention may comprise in some embodiments spacer of 11 nucleotides, a spacer of 13 nucleotides and a spacer of 24 nucleotides, as denoted by SEQ ID NO. 39, 40 and 41, respectively. Recombination can only occur between one gene coding segment flanked by a 12-RSS and another segment flanked by a 23-RSS, establishing the 12/23 rule. Because V, D, and J segments are flanked by different RSSs, the 12/23 rule helps to ensure recombination between V, D, and J, but not within homotypic gene segments. Despite the enormous specificity that RSSs confer on the recombination process, the recombination signals themselves demonstrate a remarkable degree of sequence heterogeneity. Only the first three nucleotides of the heptamer and the fifth and sixth positions of the nonamer show almost perfect conservation and are therefore thought to be the major determinants of RSS specificity and function.

The nucleic acid sequence of interest is flanked by at least one RSS. The term “flanked” as used herein refers to a nucleic acid sequence positioned between two defined regions. For example, as indicated above, the nucleic acid sequence of interest is flanked by a first and optionally, a second RSS nucleic acid sequences, where in some embodiments, the first RSS nucleic acid sequence is positioned 5′ (or upstream) to the nucleic acid sequence of interest and the optional second RSS nucleic acid sequence is positioned 3′ (or downstream) to the nucleic acid sequence of interest, and vis versa.

As referred to herein as RAG catalyzed recombination is meant that the RAG complex catalyzes two consecutive reactions, nicking (strand cleavage) and hairpin formation (strand transfer), without dissociation. First, it binds either a 12-RSS substrate or a 23-RSS substrate and introduces a nick precisely at the junction between the coding segment and the RSS. Interactions with both the conserved heptamer and nonamer are required for optimal RAG activity because considerable sequence variation in endogenous RSSs substantially affects RAG binding affinity and recombination frequency. When a 12-RSS and a 23-RSS are bound to the same RAG, a synaptic, paired complex (PC) is formed. Next, upon PC formation, the free 3′-hydroxyl released from the nicking step attacks the opposing strand to create a hairpin coding segment and a blunt signal end, generating the cleaved signal complex (CSC). Dissociation of gene segment hairpins results in a signal end complex (SEC). Proteins in the classical nonhomologous end joining (NHEJ) DNA repair pathway are recruited to the RAG complex to process and join the coding segments. High-mobility group (HMG) proteins such as HMGB1 have been shown to stimulate RAG's activity in DNA binding, nicking, and hairpin formation, presumably by inducing RSS bending.

Thus, it should be understood that in some embodiments, the mammalian target cells in addition to RAG proteins, may further express any additional protein or component that participates in the V(D)J targeted insertion provided by the methods of the invention. More specifically, in some embodiments the mammalian target cells may express in addition to RAG proteins any other protein that participate in any of the stages of the V(D)J targeted insertion described herein, to name but a few, the high-mobility group protein 1 (HMGB1) proteins, DNA dependent protein kinase (DNA-PK), the Ku dimeric protein complex, Arthemis proteins and any further relevant proteins. Still further, RAG complex as referred to herein relates in some embodiments, to RAG-1 and RAG-2 proteins. RAG-1 contains about 1000 and RAG-2 contains about 500 amino acid residues. The enzymatic activity of the RAG proteins is concentrated largely in a core region that retains most of the DNA cleavage activity. The RAG-1 core contains the DDE motif that forms the major active site for DNA cleavage. These residues are critical for nicking the DNA strand and for forming the DNA hairpin. RAG-1 comprises a nonamer-binding region (NBR) that specifically binds the conserved nonomer (9 nucleotides) of the RSS and a central domain of RAG-1 binds specifically to the RSS heptamer. The core region of RAG-2 is predicted to form a six-bladed beta-propeller structure that appears less specific than RAG-1 for its target.

In some specific embodiments, RAG-1 as used herein refers to the human RAG-1 that comprises the amino acid sequence as disclosed by AAH37344.1. In yet some further specific embodiments, the human RAG-1 comprises the amino acid sequence as denoted by SEQ ID NO. 1. In yet some further embodiments, the human RAG-1 is encoded by the nucleic acid sequence as denoted by SEQ ID NO. 2. In some further embodiments, the invention further encompasses in some embodiments thereof the mouse RAG-1 as disclosed by NP_033045.2, and as the amino acid sequence denoted by SEQ ID NO. 3. In yet some further embodiments, the mouse RAG-1 is encoded by the nucleic acid sequence as denoted by SEQ ID NO. 4.

In yet some further specific embodiments, RAG-2 as used herein refers to the human RAG-2 that comprises the amino acid sequence as disclosed by NP_001230715.1. In yet some further specific embodiments, the human RAG-2 comprises the amino acid sequence as denoted by SEQ ID NO. 5. In yet some further embodiments, the human RAG-2 is encoded by the nucleic acid sequence as denoted by SEQ ID NO. 6. The invention further encompasses in some embodiments thereof the mouse RAG-2 as disclosed by NP_033046.1, and as denoted by SEQ ID NO. 7. In yet some further embodiments, the mouse RAG-2 is encoded by the nucleic acid sequence as denoted by SEQ ID NO. 8.

As indicated above, the invention provides methods for targeted insertion of a nucleic acid sequence of interest into a target locus. As used herein, a “target locus” is a region of DNA into which a nucleic acid sequence of interest is integrated, inserted and recombined within e.g. a region of DNA in a target cell. etc. In some specific embodiments the target locus is within the chromosomal DNA of the target cell. As used herein, a “target gene” or “endogenous gene” or “gene at a target locus” is a gene that naturally exists at a locus of integration, i.e. the gene that is endogenous to the target locus. Specific target loci relevant in connection with the present invention, will be described in more detail herein after. In yet some further specific non-limiting embodiments the target loci applicable in the present invention may comprise at least one RSS, specifically, at least one RSS flanking the target site. It should be noted that in some embodiments, the at least one RSS that flank the target site may follow the role of 12/23. More specifically, in some embodiments, where the nucleic acid sequence provided by the cassette of the invention is flanked by at least one 12-RSS, the target site may be flanked by at least one 23-RSS, and vice versa. Still further, as will be elaborated in more detail herein after, in some embodiments, an appropriate target loci may be the loci coding the TCR chains (alpha, beta, gamma and delta) and the loci coding the antibody chains (IgH, IgK, IgL).

The method of the invention involves the step of contacting the nucleic acid cassette provided by the method of the invention with the target cells. The term “contacting” means to bring, put, incubate or mix together. More specifically, in the context of the present invention, the term “contacting” includes all measures or steps, which allow the positioning of the nucleic acid cassettes of the present invention such that they are in direct or indirect contact with the target cell/s.

To induced DNA integration either in vitro or in vivo, the nucleic acid cassette of the invention may be provided to and/or contacted with the target cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The nucleic acid cassette may be provided to the target cells one or more times, e.g. one time, twice, three times, or more than three times, and the cells allowed to incubate with the nucleic acid cassette for some amount of time following each contacting event e.g. 16-24 hours.

The methods of the invention involve the provision of a nucleic acid cassette. The term “nucleic acid cassette” refers to a polynucleotide sequence comprising at least one regulatory sequence operably linked to a sequence encoding a nucleic acid sequence of interest that may be a protein-encoding or non-coding sequence. In some embodiments, the nucleic acid cassettes contain at least one nucleic acid sequence/s of interest, e.g., a polynucleotide(s) of interest. In other embodiments, the nucleic acid cassette may contain one or more genetic elements e.g. expression control sequences and at least one nucleic acid sequence/s of interest. In some embodiments, the nucleic acid cassettes of the invention may be comprised within a vector. Still further, vectors may comprise one, two, three, four, five or more nucleic acid cassettes. The nucleic acid cassette may be positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. The cassette may have its 3′ and 5′ ends adapted for ready insertion into a vector, e.g., it may possess restriction endonuclease sites at each end.

As indicated above, the RSS comprised within the nucleic acid cassette provided by the methods of the invention, may be required for RAG-mediated recombination. More specifically, “mediated” as used herein encompasses any process, specifically RAG-mediated recombination that may be either directly or indirectly caused by, facilitated, enhanced, associated with and/or performed by RAG complex.

In some specific embodiments, the nucleic acid cassette used by the method of the invention, or alternatively, the nucleic acid sequence of interest comprised therein, or both, may be flanked on both the 5′ and 3′ ends thereof by RSS.

In yet some further specific embodiments, the RSS in the cassette used by the method of the invention may be at least one of 12 RSS, 23 RSS and 22 RSS. In some particular and non-limiting embodiments, RSS applicable in the present invention may by any of the RSSs as denoted by nucleic acid sequence of SEQ ID NO. 36, 37, 38, 39, 40 and 41.

In some embodiments, the RSS used by the methods and cassettes of the invention may be naturally occurring. In yet some further embodiments, the RSS used by the methods and cassettes of the invention may be selected by a screen, rationally optimized, selected based on compatibility or incompatibility with other RSSs.

In certain embodiments, the cassette provided by the method of the invention may further comprise at least one genetic element. In more specific embodiments such genetic element may be at least one of: an internal ribosome entry site (IRES), a 2A peptide coding sequence, a promoter or functional fragments thereof, a splice donor, a splice acceptor, a degron, a 3 frame stop, a protein stabilizing sequence, a signal peptide, a stop codon, a polyadenylation site, a transcription enhancer, a switch region, an mRNA stabilizing sequence and a protein stabilizing sequence.

It should be noted that in some embodiments, each of the indicated genetic elements may be located either 5′ or 3′, or both, at the 5′ and 3′ (or in other words upstream and/or downstream), to the nucleic acid sequence of interest in the cassette provided by the methods of the invention. It should be noted that the terms used herein “5′” or “upstream” and “3′” or “downstream” both refer to a relative position in DNA or RNA. Each strand of DNA or RNA has a 5′ end and a 3′ end, so named for the carbon position on the deoxyribose (or ribose) ring. By convention, upstream and downstream relate to the 5′ to 3′ direction in which RNA transcription takes place. Upstream is toward the 5′ end of the DNA or RNA molecule and downstream is toward the 3′ end. When considering double-stranded DNA, upstream is toward the 5′ end of the protein coding strand for the gene in question and downstream is toward the 3′ end. Due to the anti-parallel nature of DNA, this means the 3′ end of the mRNA template strand is upstream of the gene and the 5′ end is downstream. As used herein, the term “5′” refers to the part of the strand that is closer to the 5′ end or 5′ terminus, i.e. to the extremity of the DNA or RNA strand that has a phosphate group attached to the fifth carbon in the sugar-ring of the deoxyribose or ribose at its terminus. Furthermore, the term “3′” refers to the part of the strand that is closer to the 3′ end or 3′ terminus, i.e. to the extremity of the DNA or RNA strand that has a hydroxyl group linked to the 3rd carbon in the sugar-ring of the deoxyribose or ribose at its terminus.

As noted above, in some embodiments, the cassettes used by the methods of the invention may further comprise at least one IRES sequence.

By internal ribosome entry sequences (IRES) sequence is meant, a nucleotide sequence that allows for translation initiation in an end-independent manner, as part of the protein synthesis. IRES are able to recruit the eukaryotic ribosome to the mRNA and to provide two separate places where a ribosome may initiate translation on a single mRNA. IRES elements enable to create multigene, or polycistronic, messages since they are able to bypass the ribosome scanning model of 5′ methylated Cap dependent translation and begin translation at internal sites. Multiple open reading frames can be transcribed together, each separated by an IRES, creating polycistronic messages. By virtue of the IRES element, each open reading frame is accessible to ribosomes for efficient translation.

It should be noted that in some embodiments, the IRES sequence may be located 5′ to the nucleic acid sequence of interest. In some specific and non-limiting embodiments, an appropriate IRES that may be used by the invention may be the encephalomyocarditis virus (ECMV) IRES. In some particular and non-limiting embodiments, an ECMV IRES applicable in the present invention may comprise the nucleic acid sequence as denoted by SEQ ID NO. 27.

In yet some further embodiments, the cassette provided by the methods of the invention may further comprise at least one 2A peptide sequence. By an “internal ribosome entry site,” or “IRES” it is meant a nucleotide sequence that allows for the initiation of protein translation in the middle of a messenger RNA (mRNA) sequence. More specifically, a 2A peptide sequence or a CHYSEL site causes a eukaryotic ribosome to release the growing polypeptide chain, but continue translating, thereby giving rise to two separate polypeptides from a single translating ribosome. An expression cassette using a 2A peptide may be therefore used for two or more nucleic acid sequences of interest. In some embodiments, this sequence may be used to separate the coding region of two or more polypeptides encoded by two or more nucleic acid sequences of interest. As a non-limiting example, the sequence encoding the 2A peptide may be between a first coding region and a second coding region. In other embodiments, the 2A peptide may be used in the polynucleotides of the present invention to produce two, three, four, five, six, seven, eight, nine, ten or more proteins, or any other product of the nucleic acid sequence of interest provided by the invention. In certain embodiments, non-limiting example for 2A-peptide that may be used by the invention may be the Picornaviruse 2A peptide (P2A) In some particular and non-limiting embodiments, a P2A peptide applicable in the present invention may comprise the nucleic acid sequence as denoted by SEQ ID NO. 28.

Still further, in some embodiments, the nucleic acid cassette provide by the method of the invention may further comprise at least one promoter or any functional fragments thereof. As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

In some embodiments, promoters applicable in the present invention may be either inducible or constitutive. In yet some further embodiments, a functional fragment of a promoter applicable in the methods and cassettes of the invention may be a minimal promoter. The term “minimal promoter” includes partial promoter sequences that define the start site of transcription for the linked sequence to be transcribed which by itself is not capable of initiating transcription. Thus, the activity of such a minimal promoter is dependent upon the binding of a transcriptional activator to an operatively linked regulatory sequence, e.g., enhancer. In certain embodiments a minimal promoter may be included in the cassettes of the invention. In some specific embodiment, the minimal promoter may be a Variant of IGH minimal promoter. In some particular embodiments such minimal promoter may comprise the nucleic acid sequence as denoted by SEQ ID NO: 34. In yet some alternative embodiments, a minimal promoter applicable in the present invention may be an IgK Minimal Promoter. In more specific embodiments such minimal promoter may comprise the nucleic acid sequence as denoted by SEQ ID NO: 35.

A “constitutive promoter” refers to a promoter that allows for continual transcription of the coding sequence or gene under its control.

In yet some further embodiments, a promoter suitable in the cassette of the invention may be an inducible promoter. An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition.

It should be appreciated that the promoters suitable for the present invention may be either endogenous or heterologous. The phrase “endogenous promoter” includes a promoter that is naturally associated, e.g., in a wild-type organism, with an endogenous gene. Thus, in some specific embodiments, the cassette of the invention may comprise or operably liked to an endogenous promoter, for example, the endogenous promoter of the Ig heavy chain, Ig light chain or the TCR beta or alpha chains. It should be appreciated that such endogenous promoter may be either ectopically added or may be used in its original endogenous location.

In yet some further embodiments, the cassette of the invention may comprise heterologous promoter. The term “heterologous” includes a promoter from a different source or gene. It should be understood that in some embodiments, a promoter comprised within the nucleic acid cassette of the invention may be located 5′ to the nucleic acid sequence of interest. In some embodiments, relevant promoters that may be used by the methods and cassettes of the invention may include but are not limited to CMV promoter, SFFV promoter, EF1alpha promoter, AAT promoter, BgH promoter and any appropriate promoter.

In some embodiments, the targeted cassettes of the invention may include 2A peptide or an IRES sequence upstream to the receptor/Ab gene in order to avoid formation of protein fusions with preceding segments while still utilizing the strong endogenous promoter. The IRES may be preceded by a 3-frame stop to prevent ongoing translation. In some embodiments, the vectors may encode several peptides (e.g. two Ab chains) and these are separated by 2A peptides.

In yet some further embodiments, the cassettes provided by the invention and by the methods and compositions of the invention may further comprise at least one degron sequence. Degrons are readily understood by one of ordinary skill in the art to be amino acid sequences that control the stability of the protein of which they are part. In some embodiments, a suitable degron comprised within the nucleic acid cassette of the invention may be constitutive. In yet some further embodiments, the degron may exerts its influence on protein in an inducible manner. In some embodiments, the degron sequence may be located 5′ to the nucleic acid sequence of interest. In yet some further embodiments, the nucleic acid cassette provided by the invention and by the methods and compositions of the invention may comprise at least one signal peptide. “Signal peptide”, as used herein, shall mean a peptide chain (of about 3-60 amino acids long) that directs the post-translational transport of a protein to the endoplasmic reticulum and may be cleaved off.

In some embodiments, the signal peptide may be located 5′ to the nucleic acid sequence of interest.

In some further embodiments, the nucleic acid cassette provided by the invention and by the methods and compositions of the invention may comprise at least one mRNA stabilizing sequence. As used herein, a mRNA stabilizing sequence refers to a nucleic acid sequence that enables to extend the life-time of a mRNA strand. Non limiting examples of mRNA stabilizing elements may include Polyadenylation, 3′ untranslated regions (3′-UT) such as histone mRNA 3′-terminal stem-loop, AU-rich elements (AUREs), Iron-responsive element and Long-range stem loop of insulin-like growth factor II (IGF II), mRNA cap.

In some embodiments, the mRNA stabilizing sequence may be located 3′ to the nucleic acid sequence of interest.

In yet some further embodiments, the cassette provided by the invention and by the methods and compositions of the invention may comprise at least one stop codon. A stop codon (or termination codon) is a nucleotide triplet within messenger RNA that signals a termination of translation into proteins. Stop codons signal the termination of this process by binding release factors, which cause the ribosomal subunits to disassociate, releasing the amino acid chain. There are three different stop codons in RNA; UAG (“amber”), UAA (“ochre”), UGA (“opal”), in DNA; TAG (“amber”), TAA (“ochre”), TGA (“opal” or “umber”). It should be noted that in some embodiments, the stop codon may be located 3′ to the nucleic acid sequence of interest.

In yet some further embodiments, the cassette provided by the invention and by the methods and compositions of the invention may comprise at least one 3-frame stop codon sequence. More specifically, the cassette may comprise protein translation stop codons in each frame of translation, so that translation from the transcripts of any nucleic acid sequence of interest is halted at the point of insertion. Each translation stop sequence (known henceforth as a “3 frame stop codon sequence”) carries stop codons in all 3 frames of translation. In some embodiments, the 3 frame stop codon sequence may be located 5′ to the nucleic acid sequence of interest.

Still further, in certain embodiments, the cassette provided by the invention and by the methods and compositions of the invention may comprise a nucleic acid sequence encoding at least one protein stabilizing sequence. A protein stabilizing sequence relates to an amino acid sequence useful for stabilization of otherwise unstable proteins, particularly proteolytically sensitive proteins. The stabilization sequence may include a limited number of amino acids ranging from about ten to about 50 residues. The amino acids is such that the secondary and tertiary structure assumes the form of an outwardly directed, properly aligned hydrophobic face and a positively charged polar face. Non-limiting example of protein stabilizing sequence useful in the present invention may comprise the amino acid sequence as denoted by SEQ ID NO: 9. In some embodiments, the protein stabilizing sequence may be located 5′ to the nucleic acid sequence of interest.

Still further, in some embodiments, the cassette provided by the invention and by the methods and compositions of the invention may comprise at least one polyadenylation sequence. Polyadenylation is the addition of a poly(A) tail to a messenger RNA consisting of multiple adenosine monophosphates. In eukaryotes, polyadenylation is part of the process that produces mature messenger RNA (mRNA) for translation. The process of polyadenylation begins as the transcription of a gene terminates. The 3′-most segment of the newly made pre-mRNA is first cleaved off by a set of proteins; these proteins then synthesize the poly(A) tail at the RNA's 3′ end. The polyadenylation signal varies between groups of eukaryotes. Most human polyadenylation sites contain the AAUAAA sequence. In some embodiments, the polyadenylation sequence may be located 3′ to the nucleic acid sequence of interest.

In certain embodiments, the cassette provided by the invention and by the methods and compositions of the invention may comprise at least one splice donor site. In yet some further embodiments, the cassette provided by the methods of the invention may comprise at least one splice acceptor site. More specifically, splicing is the editing of the nascent precursor messenger RNA (pre-mRNA) transcript. After splicing, introns are removed and exons are joined together. Introns often reside within the sequence of eukaryotic protein-coding genes. Within the intron, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branch point, which includes an adenine nucleotide involved in lariat formation. In some embodiments, a splice donor (SD) may be provided downstream of the receptor/Ab gene to facilitate utilization of the endogenous C segment(s) upon integration, transcription and splicing. In particular, the SD is required to allow class switching of targeted Abs. Thus, in some embodiments, the SD may be located 3′ to the nucleic acid sequence of interest. In some specific and non-limiting embodiments, a splice acceptor (SA) applicable in the present invention may be the SA 97.66. In some specific embodiments such SA may comprise the nucleic acid sequence as denoted by SEQ ID NO: 26. In yet some further embodiments, a splice acceptor (SA) used by the invention may be the SA EF1a. More specifically, such SA may comprise the nucleic acid sequence as denoted by SEQ ID NO: 31. In some further embodiments, a splice donor (SD) that may be used by the invention may be the SD mIGHJ1/J4. In more specific embodiments, such SD may comprise the nucleic acid sequence as denoted by SEQ ID NO: 32. In yet some further embodiments a splice donor used by the invention may be the SD IgHJ3 SD 98.84. In more specific embodiments, such SD may comprise the nucleic acid sequence as denoted by SEQ ID NO: 33.

Still further, in some embodiments, the cassette provided by the invention and by the methods and compositions of the invention may comprise at least one enhancer. A transcription enhancer is a short (50-1500 bp) region of DNA that can be bound by proteins (activators) to increase the likelihood that transcription of a particular gene will occur. These proteins are usually referred to as transcription factors. Enhancers are generally cis-acting, but can also be trans-acting (acting away from the gene) and can be located up to 1 Million bp (1,000,000 bp) away from the gene and can be upstream or downstream from the start site, and either in the forward or backward direction. There are hundreds of thousands of enhancers in the human genome. The invention thus encompasses in some embodiments thereof the use of any suitable enhancer. In some embodiments, the enhancer sequence may be located 3′ to the nucleic acid sequence of interest.

It should be noted that the polynucleotide of the cassette of the invention may also comprise sequences, e.g. restriction sites, nucleotide polymorphisms, selectable markers etc., which may be used to assess for successful insertion of the gene of interest at the cleavage site. In addition, the V(D)J targeting cassette of the invention may also comprise a vector backbone containing sequences that are not homologous to the DNA region of interest and that are not intended for insertion into the DNA region of interest.

In certain embodiments, the cassette provided by the methods of the invention may comprise two or more nucleic acid sequences of interest separated by at least one genetic element, specifically, any of the gene elements disclosed above. In more specific embodiments such genetic element may be at least one of: an IRES, a 2A peptide coding sequence and a promoter and any functional fragments thereof. Thus, in some embodiments the cassettes of the invention may comprise at least two, three, four, five, six, seven, eight, nine, ten or more, fifteen, twenty, twenty five, thirty, thirty five, forty, forty five, fifty, fifty five, sixty, sixty five, seventy, seventy five, eighty, eighty five, ninety, ninety five, hundred or more nucleic acid sequences of interest separated by at least one genetic element.

As discussed above, more than one nucleic acid sequence of interest may be integrated by the V(D)J targeting methods of the invention, for example, two or more genes of interest may be integrated, three or more genes may be integrated, four or more genes may be integrated, e.g. five or more, six, seven, eight, nine, ten or more genes may be integrated into the target locus. In such instances, multiple 2A peptides or IRESs may be used to create a bicistronic or multicistronic V(D)J targeting cassette.

Alternatively, additional coding sequences of interest may be provided on the V(D)J targeting cassette under the control of a promoter distinct from that of the gene at the target locus.

In yet some further embodiments, the nucleic acid sequence of interest may be a protein coding sequence or a non-coding sequence.

A “coding sequence”, as used herein, is a DNA sequence which is transcribed and translated into a polypeptide in vivo or in vitro when placed under the control of appropriate regulatory sequences. The boundaries of the coding sequence are determined by a start codon at the 5′ (amino) terminus and a translation stop codon at the 3′ (carboxyl) terminus.

In yet some further embodiments, the nucleic acid sequence of interest may be a non-coding gene. Such gene may be transcribed in some embodiment into a non-coding RNA that will be described in more detail herein after.

In certain embodiments, the nucleic acid sequence of interest may be a protein coding nucleic acid sequence. In more specific embodiments, the nucleic acid sequence of interest may encode at least one of, a therapeutic protein or peptide, a prophylactic protein, a tolerizing protein, an immunizing protein, a toxic protein, an inducible suicide protein, a marker protein, an imaging protein and a fusion or chimeric protein.

More specifically, in some embodiments, the nucleic acid sequence of interest may encode a therapeutic and/or prophylactic protein. In some instances, the therapeutic protein alters the activity or function of the cell in which the therapeutic protein is expressed. In other words, the therapeutic protein has a cell-intrinsic effect. For example, the therapeutic and/or prophylactic protein may be an intracellular protein, transmembrane protein or secreted protein that, when expressed in a cell, will substitute for, or “complement”, a mutant protein in the cell. In other instances, the therapeutic and/or prophylactic protein alters the activity of cells other than cells in which the agent is expressed. In other words, the therapeutic and/or prophylactic protein has a cell-extrinsic effect. For example, the integrated gene of interest may encode a cytokine, chemokine, growth factor, hormone, antibody, or cell surface receptor or any chimeric or fusion protein thereof that modulates the activity of the target cell or of other cells. In yet some further embodiments, any of the proteins disclosed above may be used for the treatment and/or prophylaxis of a pathologic disorder and therefore may be referred to herein as therapeutic and/or prophylactic proteins.

In yet some further embodiments, the protein encoded by the nucleic acid sequence comprised within the cassette provided by the invention and by the methods and compositions of the invention may be an immunizing protein. An “immunizing protein” refers to a protein that elicits immunization, a process by which a subject's immune system becomes fortified against an agent (known as the immunogen). Immunizing proteins orchestrate an immune response, and also develop the ability to quickly respond to a subsequent encounter because of immunological memory: a function of the adaptive immune system.

In certain embodiments, the protein encoded by the nucleic acid sequence comprised within the cassette provided by the invention and by the methods and compositions of the invention may be a tolerizing protein. More specifically, a protein that may induce tolerization in a subject. In yet some specific embodiments, such tolerizing protein may be any peptide in the context of coagulation disorders (factor 9, factor 8, factor 7), type I diabetes (GAD, insulin, pro-insulin, HSP60, ECDISP) and Rheumatoid arthritis (DNAJ).

In some embodiments, the protein encoded by the nucleic acid sequence of interest may be a suicide gene product.

As used herein, the term “suicide gene” refers to a class of genes that produce proteins that promote the death of cells in which they expressed. Suicide genes that can be employed in the nucleic acid cassette provided by the methods of the present disclosure include the caspases, CASP3, CASP8, CASP9, BAX, DFF40 and Fas. Further non-limiting examples of suicide genes include genes that encode a peptide or polypeptide that is cytotoxic either alone or in the presence of a cofactor, e.g. a toxin such as abrin, ricin A, pseudomonas exotoxin, cholera toxin, diphtheria toxin, Herpes Simplex Thymidine Kinase (HSV-TK) and cytidine deaminase and genes that target a cell for ADCC or CDC-dependent death, e.g. CD20. It should be noted that in some embodiments a suicide gene may be a toxic gene. In some embodiments, a suicide gene may be added to the cassette which may be inducible i.e. which expression may be controlled at wish. In particular embodiments, an upstream caspase gene fused to an inducible dimerization domain (for example iCasp9) may be added to the cassette, as the default expression of iCasp9 is as an inactive monomeric caspase. In some particular and non-limiting embodiments, VDJ targeting in accordance with the invention may be employed to target the iCasp9 that comprises the nucleic acid sequence as denoted by SEQ ID NO. 17. According to the present invention, the induction of iCasp9 may depend on the administration of the small molecule dimerizer, specifically, the drug AP1903 (e.g., Drug Bank accession number DB04974) and dimerization results in rapid induction of apoptosis.

In yet some further embodiments, the protein encoded by the nucleic acid sequence of interest may be a marker protein. A marker protein may be a cell membrane-protein that serves as an identifier of the cell. A marker protein refers to a polypeptide that can determine whether or not the targeted nucleic acid cassette is expressed, therefore enabling detection. A marker protein type is not particularly limited, provided that it can be detected by a method known in the art. Examples thereof include a fluorescent protein, a pigment synthetic protein, a photoprotein, an excretory-secretory protein, and a protein that controls an external configuration. The marker proteins may enable visual detection under particular conditions without changing an external configuration.

In certain embodiments, the protein encoded by the nucleic acid sequence of interest may be an imaging protein. More specifically, the above-described marker protein may also be an “imaging protein”, i.e. a protein that is conjugated to a detectable label, which can be any molecule or agent that can emit a signal that is detectable by imaging. In some embodiments, the subject methods and compositions may be used to follow cells of interest, e.g. cells comprising an integrated nucleic acid sequence of interest. As such, the gene of interest (or one of the genes of interest) to be integrated may encode for a imaging marker. By an “imaging marker” it is meant a non-cytotoxic agent that can be used to locate and, optionally, visualize cells, e.g. cells that have been targeted by the cassettes, methods, vectors and compositions of the subject application. An imaging moiety may require the addition of a substrate for detection, e.g. horseradish peroxidase (HRP), β-galactosidase, luciferase, and the like. Alternatively, an imaging moiety may provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. a fluorescent protein. As used herein, a fluorescent protein (FP) refers to a protein that possesses the ability to fluoresce (i.e., to absorb energy at one wavelength and emit it at another wavelength). For example, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein and variants thereof.

As some further examples, the subject methods and compositions may be used to isolate cells of interest, e.g. cells comprising an integrated nucleic acid sequence of interest. Towards this end, the nucleic acid sequence of interest (or at least one of the nucleic acid sequences of interest) to be integrated may encode for a selectable marker. By a “selectable marker” it is meant an agent that can be used to select cells, e.g. cells that have been targeted by compositions of the subject application. In some instances, the selection may be positive selection; that is, the cells are isolated from a population, e.g. to create an enriched population of cells comprising the genetic modification. In other instances, the selection may be negative selection; that is, the population is isolated away from the cells, e.g. to create an enriched population of cells that do not comprise the nucleic acid sequence of interest. Any convenient selectable marker may be employed, for example, a drug selectable marker, e.g. a marker that prevents cell death in the presence of drug, a marker that promotes cell death in the presence of drug, an imaging marker, etc.; an imaging marker that may be selected for using imaging technology, e.g. fluorescence activated cell sorting; a polypeptide or peptide that may be selected for using affinity separation techniques, e.g. fluorescence activated cell sorting, magnetic separation, affinity chromatography, “panning” with an affinity reagent attached to a solid matrix, etc.; and the like.

In yet some alternative embodiments, the nucleic acid sequence of interest may be a non-coding sequence, specifically, this sequence may encode, in some non-limiting embodiments, an RNA molecule that is not translated into a protein. In yet some further specific embodiments, the nucleic acid sequence of interest may encode at least one small non-coding RNA molecule.

In yet some further embodiments, the small non-coding RNA molecule may be a molecule that control the expression of proteins, for example, an RNA molecule that participate in silencing of a target nucleic acid sequence. Suitable targets for such RNA silencing may include but are not limited to genes encoding any one of a TCR chain, a BCR chain, an immune checkpoint gene, an apoptotic gene or any gene involved in cell cycle, differentiation and survival.

In some specific embodiments, the nucleic acid sequence of the invention may encode an RNA silencing agent. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene or RNA sequence. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In some embodiments, the RNA silencing agent is capable of inducing RNA interference. In other embodiments, the RNA silencing agent is capable of mediating translational repression. More specifically, the nucleic acid sequence of interest according to the invention may encode an “antisense RNA”, which is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary” it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

In yet some specific embodiments, the nucleic acid sequence of the invention may encode an RNA, specifically, dsRNA molecule participating in RNA interference. RNA interference (RNAi), as indicated above, is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the presence of partly or perfectly double-stranded RNA (dsRNA). The silenced genes may be endogenous or exogenous to the organism, integrated into a chromosome or present in a transfection vector that is not integrated into the genome. The expression of the gene is either completely or partially inhibited.

More specifically, the dsRNA encoded by the nucleic acid sequence of interest encompassed by the invention may be selected from the group consisting of small interfering RNA (siRNA), MicroRNA (miRNA), short hairpin RNA (shRNA), PIWI interacting RNAs (piRNAs). As known in the art RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs containing a sequence complementary to one of the siRNA strands. One of the strands of the double-stranded siRNA molecule comprises a nucleotide sequence that is complementary to a nucleotide sequence of the target gene, or a portion thereof, and the second strand of the double-stranded siRNA molecule comprises a nucleotide sequence substantially similar to the nucleotide sequence of the target gene, or a portion thereof.

In more particular embodiments, siRNAs comprise a duplex, or double-stranded region, of about 18-25 nucleotides long. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product molecule as herein defined.

The strands of a double-stranded interfering RNA (e.g., siRNA) may be connected to form a hairpin or stem-loop structure (e.g., shRNA). Thus, the RNA silencing agent encoded by the nucleic acid sequence of interest according to the present invention may also be a short hairpin RNA (shRNA). The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including about 3 to 23. Some of the nucleotides in the loop can be involved in base-pair interactions with other nucleotides in the loop. According to other embodiments the RNA silencing agent encoded by the nucleic acid if interest in accordance with the invention may be a micro-RNA (miRNA). miRNAs are small RNAs made from genes encoding primary transcripts of various sizes. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target).

The pre-miRNA is a substrate for a form of dicer that removes the miRNA duplex from the precursor, after which, similarly to siRNAs, the duplex can be taken into the RISC complex.

In yet some specific embodiments, the nucleic acid sequence of interest may encode at least one of a receptor and an antibody or any fragment/s or chimera/s thereof.

In some particular embodiments, the nucleic acid sequence of interest comprised within the cassette of the invention, used and provided by the methods and compositions of the invention may encode at least one receptor. A receptor molecule as used herein refer to a molecular structure or site, specifically a protein on the surface or interior of a cell that binds with ligand substances such as hormones, antigens, drugs, or neurotransmitters. In more specific embodiments such receptor may be at least one of a T cell receptor (TCR), chimeric antigen receptor (CAR) and a B cell receptor (BCR), or any combinations thereof. In some embodiments, the nucleic acid sequence of interest may encode a receptor that may be a naturally occurring receptor, receptor selected by a screen or rationally designed receptor.

In yet some further embodiments, the protein encoded by the nucleic acid sequence comprised within the cassette provided by the methods of the invention may be a T-cell receptor (TCR). The TCR is a disulfide-linked membrane-anchored heterodimeric protein normally consisting of the highly variable alpha (a) and beta (B) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells. Each chain is composed of two extracellular domains: Variable (V) region and a Constant (C) region. The variable domain of both the TCR α-chain and β-chain each have three hypervariable or complementarity determining regions (CDRs), as well as framework regions (FRs) and a constant region. The sequence diversity of a beta T cells is largely determined by the amino acid sequence of the third complementarity-determining region (CDR3) loops of the α and β chain variable domains, which diversity is a result of V(D)J recombination. In some embodiments, a V(D)J targeting cassettes in accordance with the invention that encode a TCR may be particularly useful in targeting insertion into a target locus in T cells. In some particular embodiments, a BCR that may be used in the invention may be the ESO-1-specific TCR. In more specific embodiments, such TCR may comprise the nucleic acid as denoted by SEQ ID NO: 12.

In some further embodiments, the protein encoded by the nucleic acid sequence comprised within the cassette provided by the methods of the invention may be a B-cell receptor (BCR). The B-cell receptor or BCR is a transmembrane receptor protein located on the outer surface of B cells. The B-cell receptor is composed of two elements, specifically, (i) a membrane-bound immunoglobulin molecule of one isotype (IgD, IgM, IgA, IgG, or IgE) with the exception of the presence of an integral membrane domain, these are identical to their secreted forms; and (ii) a signal transduction moiety composed of a heterodimer called Ig-α/Ig-β (CD79), bound together by disulfide bridges. Each member of the dimer spans the plasma membrane and has a cytoplasmic tail bearing an immunoreceptor tyrosine-based activation motif (ITAM). It should be appreciated that in some embodiments, a cassette encoding a BCR may be particularly useful in targeting insertion into a target locus in B cells. In some particular embodiments, a BCR that may be used in the invention may be the anti-polio BCR. In more specific embodiments, such BCR may comprise the nucleic acid as denoted by SEQ ID NO: 23. In yet some further particular embodiments, a BCR that may be used by the invention may be the anti-Respiratory syncytial virus (RSV) BCR. In some specific embodiments, such BCR may comprise the nucleic acid as denoted by SEQ ID NO: 24.

It should be noted that “receptor” as used herein also encompasses any variant, chimeric or fusion protein of any of the receptors described herein, specifically, TCR or BCR chimeric or fusion proteins. Cancer immunotherapy aims to augments immune mechanisms to fight malignancies. In particular, adoptive T cell transfer entails the activation and expansion of T cells that target tumor associated antigens (TAA). Naturally occurring T cell receptors (TCRs) often have low affinity against TAAs. However, T cells can be engineered to express highly potent TCRs, which can recognize fragments of both intracellular and cell surface proteins when presented in a major histocompatibility complex (MHC) context. Alternatively, T cells can be engineered to express chimeric antigen receptors (CARs), which have a high TAA affinity. As used herein, the term “fusion or chimeric protein” refers to a recombinant protein in which two or more proteins or domains responsible for a specific function within a protein are linked so that each protein or domain is responsible for its intrinsic function. A linker having a flexible structure may conventionally be inserted between the two or more proteins or domains.

In some further embodiments, the protein encoded by the nucleic acid sequence comprised within the cassette provided by the methods of the invention may be a Chimeric antigen receptor (CAR). CAR, as used herein, relates to artificial T cell receptors (also known as chimeric T cell receptors, chimeric immuno-receptors). These are engineered receptors, which graft an arbitrary specificity onto an immune effector cell. Typically, these receptors are used to graft the specificity of a monoclonal antibody onto a T cell.

The initial design (also referred to a first generation) joined an antibody-derived scFv to the CD35 intracellular signaling domain of the T-cell receptor through hinge and transmembrane domains.

Second generation CARs add intracellular signaling domains from various costimulatory protein receptors (e.g., CD28, 41BB, ICOS) to the cytoplasmic tail of the CAR to provide additional signals to the T cell. More recent, third generation CARs combine multiple signaling domains, such as CD27, CD28, 4-1BB, ICOS, or OX40, to augment potency.

CD19 is a B-cell surface protein expressed throughout B-cell development; therefore, it is expressed on nearly all B-cell malignancies, including chronic lymphocytic leukemia (CLL), ALL, and many non-Hodgkin lymphomas. This near-universal expression and specificity for a single cell lineage has made CD19 an attractive target for CAR-modified T-cell therapies. Additional B-cell-specific cell-surface molecules, such as CD22, may hold similar promise and are under active investigation, and may therefore be also applicable in the present invention.

Thus, in some particular and non-limiting embodiments, VDJ targeting may be employed to target an anti CD19 CAR coding nucleic acid sequence which are highly effective in treating B cell malignancies. In yet some further specific embodiments, VDJ targeting according to the invention may be employed to target the nucleic acid sequence that encodes the anti CD19 CAR, specifically, such anti CD19 CAR may comprise in some embodiments, the amino acid sequence as denoted by SEQ ID NO 10 or any homologs, variants or derivatives thereof. Thus, in some embodiments, the cassettes provided by the invention and used by the methods and compositions discussed herein may comprise as at least one nucleic acid sequence of interest, a nucleic acid sequence encoding the polypeptide as denoted by SEQ ID NO. 10.

In yet some further specific embodiments, VDJ targeting in accordance with the invention may be employed to insert an anti-Tn-MUC1 CAR coding nucleic acid sequence which targets a highly cancer specific glycoform. Aberrant expression of Tn glycoforms (GalNAc-Ser/Thr) have in particular been found on the cell membrane mucin MUC1, which is a large protein with tandem repeated sequences carrying O-glycans overexpressed in most adenocarcinomas. In health, the Tn antigen is not expressed and humans have natural anti-Tn IgM antibodies. However, exposure of Tn in cancer cells may lead to loss of immunological tolerance to Tn-glycopeptide epitopes, induction of IgG antibodies and immunopathology. In some particular and non-limiting embodiments, the CAR-ANTI-Tn-MUC1 may be encoded a nucleic acid sequence comprising the nucleic acid sequence as denoted by SEQ ID NO. 11. Thus, in some embodiments, the VDJ targeting according to the invention may be employed to target the nucleic acid sequence as denoted by SEQ ID NO. 11 that encodes the CAR-ANTI-Tn-MUC1, or any homologs, variants or derivatives thereof. Thus, in some embodiments, the cassettes provided by the invention and used by the methods and compositions discussed herein may comprise as at least one nucleic acid sequence of interest, a nucleic acid sequence encoding the polypeptide as denoted by SEQ ID NO. 11. In some embodiments, VDJ targeting may be employed for targeted insertion of an NY-ESO-1-specific TCR construct. NY-ESO-1 (also known as CTAG-1B) is an immunogenic cancer testis antigen (CTA) associated with spontaneous and vaccine-induced immunity that can lead to clinical cancer responses. Up to 60% of advanced myelomas have been reported to express NY-ESO-1, a feature correlated to tumor proliferation and high risk features. Thus, in certain specific embodiments, VDJ targeting according to the invention may be employed to target the nucleic acid sequence that encodes the anti-NY-ESO-1-TCR. In more specific embodiments, the NY-ESO-1-TCR may comprise the amino acid sequence as denoted by SEQ ID NO 12. Thus, in some embodiments, the cassettes provided by the invention and used by the methods and compositions discussed herein may comprise as at least one nucleic acid sequence of interest, a nucleic acid sequence encoding the polypeptide as denoted by SEQ ID NO. 12.

The nucleic acid sequence of interest comprised within the cassette provide by the invention may encode a protein or polypeptide as described herein above (e.g., receptors or antibodies). The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the invention encompasses the use of any variant or derivative of the polypeptides of the invention and any polypeptides that are substantially identical or homologue to the polypeptides encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically, receptors, chimeras and antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the proteins that comprise the amino acid sequence as denoted by SEQ ID NO. 10, 11 and 12, as well as the BCRs sequences as denoted by SEQ ID NO. 23 and 24 and the antibodies sequences described herein after, specifically the sequences as denoted by SEQ ID NO. 13, 14, 15 and 16. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 10, 11, 12, 13, 14, 15, 16, 23, 24 specifically, the entire sequence as denoted by SEQ ID NO. 10, 11, 12, 13, 14, 15, 16, 23, 24.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

Thus, in some embodiments, the invention encompasses targeting of any nucleic acid sequence of interest that encodes any of the specified polypeptides (e.g., receptors, chimeric receptors and antibodies), or any derivatives thereof, specifically a derivative that comprise at least 1, 2, 3, 4, 5, 6, 7, 8, 9, 10 or more conservative substitutions to the amino acid sequences as denoted by any one of SEQ ID NO. 10, 11, 12, 13, 14, 15, 16, 23, 24. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine(S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention.

In yet some alternative embodiments, the nucleic acid sequence of interest comprised within the cassette of the invention may encode at least one antibody or any antibody binding domains, fragments or portions thereof. In more specific embodiments, such antibody may be at least one of a full length antibody, antibody fragment, single-chain variable fragment (scFv), bi-specific antibody, tri-specific antibody, Bi-specific T-cell engagers (BiTE) and variable new antigen receptor antibody (V-NAR).

In some embodiments, the nucleic acid sequence of interest may encode an antibody that may be a naturally occurring antibody, antibody or fragments thereof selected by a screen or rationally designed antibody.

Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics. Still further, as indicated above, an “antigen-binding domain” can comprise or consist of an antibody or antigen-binding fragment of an antibody. The term “antibody” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The antibody suitable for the invention may also be a bi-specific antibody (such as Bi-specific T-cell engagers-BiTEs) or a tri-specific antibody.

The antibody suitable for the invention may also be a variable new antigen receptor antibody (V-NAR). VNARs are a class of small, immunoglobulin-like molecules from the shark immune system. Humanized versions of VNARs could be used to bind protein epitopes that are difficult to access using traditional antibodies.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

It should be noted that in some specific embodiments, the receptor or antibody encoded by the nucleic acid sequence of interest of the cassette of the invention may be directed at or specific for a tumor associated antigen (TAA).

Tumor or cancer associated antigen, as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present invention, include, but are not limited to, RAGE-1, tyrosinase, MAGE-1, MAGE-2, NY-ESO-1, Melan-A/MART-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph. and TRP-1. Still further, TAA may be recognized by CD8+ T cells as well as CD4+ T cells. Non limiting examples of TAA recognized by CD8+ T cells may be CSNK1A1, GAS7, HAUS3, PLEKHM2, PPP1R3B, MATN2, CDK2, SRPX (P55L), WDR46 (T227I), AHNAK (S4460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PASSA WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+ T cells may be ERBB2IP (E805G), CIRH1A (P333L), GART (V551A), ASAP1 (P941L), RND3 (P49S), LEMD2 (P495L), TNIK (S502F), RPS12 (V1041), ZC3H18 (G269R), GPD2 (E426K), PLEC (E1179K), XPO7 (P274S), AKAP2 (Q418K) and ITGB4 (S10021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph.

Cancer antigen and tumor antigen are used interchangeably herein. The antigens may be related to cancers that include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer). In some particular embodiments, antibodies applicable in the methods, encoded by the cassettes and compositions of the invention may include but are not limited to the 3BNC117 antibody, an anti-TNFα antibody, specifically, the Adalimumab (Humira™) antibody, anti MUC1 glycoform antibody, specifically, the PankoMab-GEX. In some particular and non-limiting embodiments such specific antibodies may comprise the amino acid sequence as denoted by SEQ ID NOs. 13, 14 and 15 and 16, respectively or any homolog, variant or derivative thereof as disclosed herein before. Thus, in some embodiments, the cassettes provided by the invention and used by the methods and compositions described herein may comprise as an at least one nucleic acid sequence of interest, a nucleic acid sequence encoding the antibodies that comprise an amino acid sequence as denoted by any one of SEQ ID NOs. 13, 14 and 15 and 16.

In some particular embodiments, the nucleic acid cassette may comprise a nucleic acid sequence encoding a secreted antibody directed against and specific for an autoantigen and in addition, a BCR directed against and specific for a foreign antigen. In yet some further specific embodiments, the antibody and the BCR encoding sequences are separated by at least one of, IRES, a 2A peptide coding sequence and/or a promoter.

An “autoantigen” as used herein, refers to an endogenous antigen that despite being a normal tissue constituent, evokes an immune response by the host.

The term “foreign antigen” refers to any antigens and epitopes that are not naturally a part or a component of the subject but instead, may be in some embodiments a part of an organism or pathogen that infects the subject and causes a disease or disorder. Therefore, foreign antigens may be any infectious agent such as a bacterium, a prion, a parasitic agent, a eukaryotic unicellular or multicellular infectious agent, a fungus or a virus. The virus may be any virus, such as poliovirus, poxvirus, smallpox virus, herpes virus, retrovirus, HIV, measles virus, rubella virus, rhinovirus, yellow fever virus, dengue virus, hepatitis viruses A, B, or C, rabies virus, rhabdovirus or any other virus.

In the context of the invention, a foreign antigen may be used as a “foreign inducer” to induce secretion of an autoantibody. In some specific embodiments, as disclosed by the following examples, specifically Example 5, the targeting cassette of the invention may separately encode a secreted antibody directed against a disease associated auto-antigen and a BCR directed against the foreign inducer. The targeted B cell differentiates to Antibody-secreting plasma cells only upon administration of the foreign inducer.

In some embodiments, the foreign inducer of BCR activation may be an antigen of a known vaccine. A prominent example may be the Polio vaccine, a safe, cheap, that is rarely encountered through the natural life course, thus diminishing the risk of spurious activation. There are two types of Polio vaccines: one that uses inactivated poliovirus and is given by injection (IPV), and one that uses weakened poliovirus which is orally administered (OPV). It should be appreciated that both types may be applicable in the present invention.

In yet some other specific embodiments applicable in the present invention, an autoantigen may be Her2 (=Neo=Erbb2) and the BCR may be Pertuzumab or Trastuzumab (with the added transmembrane domain from IgH).

In certain embodiments, the target genomic locus targeted by the method of the invention may be at least one of Immunoglobulin heavy chain locus, Immunoglobulin κ chain locus, Immunoglobulin λ chain locus, TCRβ chain locus, TCRα chain locus, TCRγ chain and the TCRδ chain locus. More specifically, in some embodiments, such target locus is a locus where a natural V(D)J recombination occurs naturally, specifically, V(D)J recombination mediated by RAG complex.

It should be noted that “target genomic locus” is the site of modification of an endogenous chromosomal locus by the insertion into, integration into, deletion of, or replacement of the endogenous sequence via V(D)J recombination using the cassette of the invention.

T cell receptors are composed of an alpha chain and a beta chain. More specifically, TCRβ chain locus, TCRα chain locus, TCRγ chain locus and the TCRδ chain locus refers to the chromosomal position of the TCRβ chain coding gene (on chromosome 7), of the TCRα chain coding gene (on chromosome 14), of the TCRγ chain coding gene (on chromosome 7) and of the TCRδ chain coding gene (on chromosome 14), respectively. The κ segments are on human chromosome 2 and the λ segments are on human chromosome 22. As noted herein before, the T cell receptor genes are similar to immunoglobulin genes since they contain multiple V, D and J gene segments in their beta chains (and V and J gene segments in their alpha chains) that are rearranged during the development of the lymphocyte to provide a unique antigen receptor. The TCR comprises 50 Vα, 50 Jα, 20 Vβ, 13 Jβ, 2 Dβ gene segments (100 α, 35 β), and a smaller number of γδ gene segments. Thus, the target genomic locus may be located at any of these specific segments. A typical antibody is composed of two immunoglobulin (Ig) heavy chains and two Ig light chains. In humans, antibodies are encoded by three independent gene loci, namely the immunoglobulin heavy locus (IgH) on chromosome 14, containing the gene segments for the immunoglobulin heavy chain, the immunoglobulin kappa (κ) locus (IgK) on chromosome 2, containing the gene segments for part of the immunoglobulin light chain and the immunoglobulin lambda (λ) locus (IgL) on chromosome 22, containing the gene segments for the immunoglobulin light chain.

The antibody and BCR heavy chains comprises 51 Variable (V) gene segments, 27 Diversity (D) gene segments, 6 Joining (J) gene segments. The antibody and BCR light chains comprises 40 Vκ, 31 Vλ, 5 Jκ, 4 Jλ gene segments.

In yet some further embodiments, the cassette provided by the methods of the invention may be comprised within a nucleic acid vector. In more specific embodiments, such vector may be any one of a viral vector, a non-viral vector and a naked DNA vector.

Vectors, as used herein, are nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, etc., useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as AAV, MMLV, HIV-1, ALV, etc.

Vectors may be provided directly to the subject cells. In other words, the cells are contacted with vectors comprising the cassettes of the invention that comprise the nucleic acid sequence of interest such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV).

More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

As indicated above, in some embodiments, viral vectors may be applicable in the present invention. The term “viral vector” refers to a replication competent or replication-deficient viral particle which are capable of transferring nucleic acid molecules into a host.

The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e. vectors constructed using complementary coding sequences from more than one viral subtype.

In some embodiments, the cassette of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells.

Production systems for rAAV vectors typically consist of a DNA-based vector containing a transgene expression cassette, which is flanked by inverted terminal repeats. Construct sizes are limited to approximately 4.7-5.0 kb, which corresponds to the length of the wild-type AAV genome. rAAVs are produced in cell lines. The expression vector is cotransfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments AAV serotype 6 may be suitable for the methods of the invention. In yet some further embodiments, AAV serotype 8 may be suitable for the methods of the invention. In some embodiments, the AAV serotype 6 may be encoded by the nucleic acid sequence as denoted by GenBank accession number AF028704.1. In some specific embodiments, the AAV serotype 6 may be encoded by the nucleic acids sequence as denoted by SEQ ID NO: 29. In some embodiments, the AAV serotype 8 may be encoded by the nucleic acid sequence as denoted by GenBank accession number NC_006261.1. In some specific embodiments, the AAV serotype 8 may be encoded by the nucleic acids sequence as denoted by SEQ ID NO: 30.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB, but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the methods of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long term expression of the transgene.

Still further, in some embodiments, SV40 may be used as a suitable vector by the methods of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10 (12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the cassette with the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the cassette of the invention that contains the nucleic acids sequence of interest into packaging cell lines and of collecting the viral particles that are generated by the packaging lines are well known in the art.

In some alternative embodiments, the vector may be a non-viral vector. More specifically, such vector may be in some embodiments any one of plasmid, minicircle and linear DNA.

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA.

Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or Doggybone™ are double-stranded, linear DNA construct that solely encodes an antigen expression cassette, comprising antigen, promoter, polyA tail and telomeric ends.

It should be appreciated that all DNA vectors disclosed herein, may be also applicable in the methods, cassettes and compositions of the invention.

Still further, it must be appreciated that the invention further provides any vectors or vehicles that comprise any of the nucleic acid cassettes disclosed by the invention, as well as any host cell expressing the nucleic acid cassettes disclosed by the invention.

In some specific embodiments, the cassette of the invention introduces the nucleic acid of interest in a target locus of a mammalian cell that is considered herein as a host cell. The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the term “host cell”. It should be noted that the host cell of the invention express at least one of RAG proteins.

In yet some further embodiments, the method of the invention is directed at targeted insertion of a nucleic acid sequence of interest in a target locus of a mammalian cell. In some specific embodiments, the mammalian cells may be any one of a naturally RAG expressing cell, stably RAG expressing cell, inducibly RAG expressing cell and a cell transfected with RAG expression vector/s. It should be noted that RAG expressing cells in some embodiments may be cells that actively undergo RAG mediated VDJ recombination. However, it should be understood that “RAG expressing cells” targeted by the methods of the invention may also encompass in some embodiments cells that have already underwent VDJ recombination but still express RAG. More specifically, in some embodiments, such cells no longer undergo VDJ recombination at the V locus, but are still permissive for VDJ targeting at their D and J loci. In some embodiments, such integration may allow expression if the transgene is preceded by at least one of a promoter of any fragments thereof (e.g., a minimal promoter) and/or a splice acceptor.

In more specific embodiments, the target cell may be an immune cell, specifically hematopoietic cell. In yet some further specific embodiments, such hematopoietic cell may be of the lymphoid lineage, specifically, a lymphocyte. “Lymphocytes” are mononuclear nonphagocytic leukocytes found in the blood, lymph, and lymphoid tissues. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells. In yet more specific embodiments, such cell may be any one of a lymphocyte progenitor, ex vivo differentiating lymphocytes, hematopoietic stem and progenitor cells (HSPCs) and Induced pluripotent stem cells (iPSCs).

It should be appreciated that in some embodiments, the target cells may be pluripotent cells, specifically, hematopoietic pluripotent cells. The term “pluripotent” refers to cells with the ability to give rise to progeny that can undergo differentiation, under the appropriate conditions, into cell types that collectively demonstrate characteristics associated with different cell lineages.

In more specific embodiments, cells suitable in the present invention may be iPSCs. As used herein, “induced pluripotent stem cells” or “iPSCs” refers to a type of pluripotent stem cell artificially derived from a non-pluripotent cell, typically an adult somatic cell, by inducing a “forced” expression of certain genes.

Still further, in some embodiments, cells suitable in the present application may be immobilized HSP. Hematopoietic stem cells (HSCs) normally reside in the bone marrow but can be forced into the blood, a process termed bone marrow mobilization used to harvest large numbers of HSCs in peripheral blood. One mobilizing agent of choice in accordance with the invention may be granulocyte colony-stimulating factor (G-CSF). The resulting HSCs are further referred as mobilized HSCs.

More specifically, hematopoietic stem cells (HSCs) originate from the bone marrow. They first differentiate into multipotent progenitor (MPP) cells, and then may differentiate to common lymphoid progenitor (CLP) cells.

A “committed lymphoid progenitor cell” refers to an oligopotent or unipotent progenitor cell capable of differentiating into any of the terminally differentiated cells of the lymphoid lineage, such as T cell, B cell, NK cell, or lymphoid dendritic cells. Different cell populations of lymphoid progenitors are distinguishable from other cells by their differentiation potential, and the presence of a characteristic set of cell markers. Encompassed within the lymphoid progenitor cells are the “common lymphoid progenitor cells (CLP)”, which are oligopotent cells characterized by a capacity to give rise to B-cell progenitors (BCP), T-cell progenitors (TCP), NK cell progenitors, and dendritic cells. These progenitor cells have little or no self-renewing capacity, but are capable of giving rise to T lymphocytes, B lymphocytes, NK cells, and lymphoid dendritic cells.

More specifically, in some embodiments, the lymphocyte progenitor may be any one of a T cell progenitor, a B cell progenitor and an NK cell progenitor.

A “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells.

More specifically, Thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Thymocytes are classified into a number of distinct maturational stages based on the expression of cell surface markers. The earliest thymocyte stage is the double negative (DN) stage (negative for both CD4 and CD8), which more recently has been better described as Lineage-negative, and which can be divided into four sub-stages. The next major stage is the double positive (DP) stage (positive for both CD4 and CD8). The final stage in maturation is the single positive (SP) stage (positive for either CD4 or CD8).

More specifically, the maturational stages of thymocytes may include the following substages: Double negative 1 (DN1) or ETP (Early T lineage Progenitor) is characterized by CD44+CD25-CD117+ defining surface markers, thymocytes are located in the cortex and proliferation, loss of B and myeloid potentials are observed; Double negative 2 (DN2) is characterized by CD44+CD25+CD117+ defining surface markers and thymocytes are located in the cortex; Double negative 3 (DN3) is characterized by CD44−CD25+ defining surface markers, thymocytes are located in the cortex and TCR-beta rearrangement and beta selection are observed; Double negative 4 (DN4) is characterized by CD44−CD25-defining surface markers and thymocytes are located in the cortex; Double positive is characterized by CD4+CD8+ defining surface markers, thymocytes are located in the cortex and TCR-alpha rearrangement, positive selection, negative selection are observed; Single positive is characterized by CD4+CD8− or CD4−CD8+ defining surface markers, thymocytes are located in the medulla and Negative selection is observed.

In human, circulating CD34+ hematopoietic stem cells (HSC) reside in bone marrow. They produce precursors of T lymphocytes, which seed the thymus (thus becoming thymocytes) and differentiate under influence of the Notch and its ligands. Early, double negative thymocytes express (and can be identified by) CD2, CD5 and CD7. Still during the double negative stage, CD34 expression stops and CD1 is expressed. Expression of both CD4 and CD8 makes them double positive, and matures into either CD4+ or CD8+ cells.

Still further, in some embodiments, the hematopoietic cell applicable in the methods and compositions of the invention may be a B cell progenitor. B cells develop from HSCs that originate from bone marrow. Their development into B cells occurs in several stages, each marked by various gene expression patterns and immunoglobulin H chain and L chain gene loci arrangements, the latter due to B cells undergoing V(D)J recombination as they develop.

To ensure proper development, B cells undergo two types of selection while developing in the bone marrow. Positive selection occurs through antigen-independent signaling involving both the pre-BCR and the BCR. If these receptors do not bind to their ligand, B cells do not receive the proper signals and cease to develop. Negative selection occurs through the binding of self-antigen with the BCR; if the BCR can bind strongly to self-antigen, then the B cell undergoes one of four fates: clonal deletion, receptor editing, anergy, or ignorance (B cell ignores signal and continues development). This negative selection process leads to a state of central tolerance, in which the mature B cells does not bind with self-antigens present in the bone marrow.

To complete development, immature B cells migrate from the bone marrow to the spleen as well as pass through two transitional stages: T1 and T2. Throughout their migration to the spleen and after spleen entry, they are considered T1 B cells. Within the spleen, T1 B cells transition to T2 B cells. T2 B cells differentiate into either follicular (FO) B cells or marginal zone (MZ) B cells depending on signals received through the BCR and other receptors. Once differentiated, they are now considered mature B cells, or naive B cells.

While immature and during the T1 phase, B cells express BCR of class IgH, but BCR expression changes to the classes IgM and IgD after transition into the T2 phase and while mature up to activation.

It should be appreciated that in some specific embodiments, the targeted insertion of at least one nucleic acid sequence of interest into a target genomic locus in a target cell may be performed by the method of the invention in a mammalian subject. It is to be understood that the invention therefore provides in some aspects thereof any host cell transfected, transformed or transduced by any of the vectors or vehicles of the invention that comprise any of the nucleic acid cassettes disclosed herein. Such cell may be any of the cells, specifically the hematopoietic cells disclosed by the invention.

In a second aspect, the invention relates to a method for targeted insertion of at least one nucleic acid sequence of interest into a target genomic locus of a cell in a mammalian subject. More specifically, the method may comprise the step of administering to the subject an effective amount of at least one nucleic acid cassette comprising the nucleic acid sequence of interest and at least one RSS. Alternatively, a vector comprising the cassette of the invention may be administered to the subject. It should be noted that the nucleic acid cassette administered by the method of the invention enables the insertion of a nucleic acid sequence of interest into a target genomic locus. More specifically, such insertion may be facilitated, mediated and/or performed by RAG-catalyzed recombination between at least one genomic RSS flanking the target genomic locus and at least one RSS comprised within the nucleic acid cassette.

In some specific embodiments the nucleic acid cassette may be flanked on both the 5′ and 3′ ends thereof by RSS.

In more specific embodiments, the RSS comprised within the cassette administered by the method of the invention may be at least one of 12 RSS, 23 RSS and 22 RSS. In some particular and non-limiting embodiments, RSS applicable in the present invention may by any of the RSSs as denoted by nucleic acid sequence of SEQ ID NO. 36, 37, 38, 39, 40 and 41.

In some embodiments, the RSS used by the methods and cassettes of the invention may be naturally occurring, selected by a screen, rationally optimized, selected based on compatibility or incompatibility with other RSSs.

In yet some further specific embodiments, the nucleic acid cassette administered by the method of the invention may further comprise at least one genetic element, said genetic element is at least one of: an IRES, a 2A peptide coding sequence, a promoter or any functional fragment thereof, a splice donor, a splice acceptor, a degron, a 3 frame stop, a protein stabilizing sequence, a signal peptide, a stop codon, a polyadenylation site, a transcription enhancer, a switch region, an mRNA stabilizing sequence and a protein stabilizing sequence.

In some specific embodiments, the cassette administered by the method of the invention may comprise two or more nucleic acid sequences of interest separated by at least one genetic element. In more specific embodiments, such genetic element may be at least one of an IRES, a 2A peptide coding sequence and a promoter.

In certain embodiments, the nucleic acid sequence of interest comprised within the cassette administered by the method of the invention may be a protein coding nucleic acid sequence or a non-coding sequence.

In some specific embodiments, where the nucleic acid sequence of interest is a protein coding gene, such protein may be at least one of: a therapeutic protein or peptide, a prophylactic protein, a tolerizing protein, an immunizing protein, a toxic protein, an inducible suicide protein, a marker protein, an imaging protein and a fusion or chimeric protein.

In yet some further alternative embodiments, the nucleic acid sequence of interest may encode at least one small non-coding RNA molecule. More specifically, such one small non-coding RNA molecule may be for example, miRNA, siRNA, shRNA or any of the one small non-coding RNA molecules described by the invention herein before. In some further embodiments, the small non-coding RNA molecule may be specifically directed against a TCR chain, a BCR chain, an immune checkpoint gene or an apoptotic gene.

In more specific embodiments, the nucleic acid sequence of interest of the cassette administered by the method of the invention may encode at least one of a receptor and an antibody or any fragment/s or chimera/s thereof.

In some particular embodiments, the nucleic acid sequence of interest may encode at least one receptor. More specifically, such receptor may be any one of: a TCR, CAR and a BCR.

In some embodiments, the nucleic acid sequence of interest may encode a receptor that may be a naturally occurring receptor, receptor selected by a screen or rationally designed receptor.

In yet some further particular embodiments, the nucleic acid sequence of interest may encode at least one antibody. More specifically, such antibody may be any one of full length antibody, antibody fragment, scFv, bi-specific antibody, tri-specific antibody, BiTE and V-NAR.

In some embodiments, the nucleic acid sequence of interest may encode an antibody that may be a naturally occurring antibody, antibody or fragments thereof selected by a screen or rationally designed antibody.

In some embodiments, the nucleic acid cassette administered by the method of the invention may comprise a nucleic acid sequence encoding a secreted antibody directed against and specific for an autoantigen. This cassette may further comprise a nucleic acid sequence encoding a BCR directed against and specific for a foreign antigen. It should be noted that in certain embodiments, these two nucleic acid sequences are separated by at least one genetic element, for example, an IRES, a 2A peptide coding sequence and a promoter.

In certain embodiments, the target genomic locus for the targeted insertion of the nucleic acid sequence of interest comprised within the cassette administered by the method of the invention may be at least one of Immunoglobulin heavy chain locus, Immunoglobulin κ chain locus, Immunoglobulin λ chain locus, TCRβ chain locus, TCRα chain locus, TCRγ chain and the TCRδ chain locus.

It should be appreciated that in certain embodiments, the nucleic acid cassette administered by the method of the invention may be comprised within a nucleic acid vector. More specifically, such vector may be any one of a viral vector, a non-viral vector and a naked DNA vector.

In more specific embodiments, the vector may be a viral vector. Specifically, suitable viral vectors may be any one of rAAV, ssAAV, scAAV, SV40 vector, Adeno virus vector, helper-dependent Adeno viral vector, retroviral vector and lentiviral vector.

In yet some further embodiments, the cassette administered by the method of the invention may be comprised within a non-viral vector. Such vector may be any one of plasmid, minicircle and linear DNA. In yet some further embodiments, the vector may be a naked DNA vector, specifically, any one of plasmid, minicircle and linear DNA.

It should be noted that in certain embodiments, the cassette administered by the method of the invention to a subject, leads to targeted insertion of the nucleic acid sequence of interest into a target locus in a cell of said mammalian subject. It should be noted that the targeted insertion may be mediated and facilitated by RAG complex. Thus, in some specific embodiments, the targeted cell/s of the subject may naturally express RAG. However, it should be noted that the invention further encompasses in some embodiments thereof, the option that the target cells of the administered subject may be either stably RAG expressing or inducibly RAG expressing. Such cells in some embodiments may be transfected with RAG expression vectors. As indicated herein before, in some embodiments, RAG expressing cells may be cells that undergo VDJ recombination. In yet some further embodiments, RAG expressing cell may further encompass cells that already underwent VDJ recombination but still express RAG.

In certain embodiments, the cell/s of the administered subject targeted by the cassette of the invention may be any one of thymocytes, HSPCs and mobilized HSPCs.

In yet some specific embodiments, the cells targeted by the cassette administered by the method of the invention may be thymocytes of any subsect. More specifically, such cells may be thymocytes of any one of the DN1, DN2, DN3, DN4, DP and SP subsets, as described herein above. In yet some further embodiments, the cells targeted by the cassette administered to the subject by the method of the invention may be splenocytes of any subsect, or any lymphocytes obtained or present in lymph nodes and bone marrow.

The subject methods and compositions may be applied to any disease, disorder, or natural cellular process that would benefit from modulating cell activity by integrating a gene of interest.

In some specific embodiments, the subject administered by the method of the invention may be a subject in need. In more particular and non-limiting embodiments, such subject may be a subject suffering of an immune-related disorder.

It should be further appreciated that the method of the invention also encompasses the option that the targeted insertion of the nucleic acid sequence of interest to cells of the subject may be performed ex vivo, to cells of the subjects (cells of autologous source) or alternatively, to cells of allogeneic source. These cells may be than administered to the subject by adoptive transfer. The term “adoptive transfer” as herein defined applies to all the therapies that consist of the transfer of components of the immune system, specifically cells that are already capable of mounting a specific immune response. Examples of adoptive transfer include both the transfer of antibodies and also, in adoptive cell transfer, specific types of cells that are capable of mediating antigen-specific tumor regression such as T cells.

A further aspect of the invention relates to a method for treating, preventing, ameliorating, inhibiting or delaying the onset of a pathologic disorder in a mammalian subject. More specifically, the method of the invention may comprise the step of administering to the treated subject an effective amount of at least one of: (a) nucleic acid cassette for targeted insertion of at least one nucleic acid sequence of interest into a target genomic locus; (b) a vector comprising said nucleic acid cassette; and (c) a cell transduced or transfected with the nucleic acid cassette, or any combinations thereof. It should be noted that the cassette administered by the method of the invention may comprise at least one nucleic acid sequence of interest and at least one RSS. It should be understood that in some embodiments, the insertion of the nucleic acid sequence of interest into the target genomic locus in cell/s of the treated subject may be facilitated, mediated and/or performed by RAG-catalyzed recombination between at least one genomic RSS flanking the target genomic locus and at least one RSS comprised within the nucleic acid cassette.

Thus, the invention provides therapeutic and prophylactic methods that are based on targeted insertion of a nucleic acid sequence of interest into a specific locus in cells of a subject in need. It should be appreciated that the methods of the invention enable whether in vivo insertion of the nucleic acid sequence of interest into cells of the treated subjects, by administering to the treated subject the nucleic acid cassette, or a vector comprising said cassette. In some alternative embodiments, the insertion of at least one nucleic acid sequence of interest into a specific locus in cells of the subject, may be performed ex vivo. In such option, the targeted insertion of the nucleic acid sequence of interest is performed in cells of an autologous or allogeneic source, that are then administered to the subject, specifically, by adoptive transfer.

In some embodiments, the subject treated by the method of the invention undergoes or may be subjected to a preceding treatment. In some specific embodiments, such preceding treatment may be at least one of HSPC mobilization, immune ablation and steroid administration.

In some embodiments, the treated patient treated by the method of the invention may be subjected to preceding treatment. In some embodiments, such treatment may be bone marrow mobilization. Bone marrow mobilization of progenitor cells is a multistage process, with initial release from their bone marrow niche followed by active migration across the bone marrow sinusoidal endothelium. The chemokine axis SDF-1α/CXCR4 is critically involved in the retention of hematopoietic stem cells within the bone marrow. At a molecular level, G-CSF acts by disrupting the SDF-1α/CXCR4 retention axis, both by reducing CXCR4 expression on HPCs and levels of SDF-1α in the bone marrow.

CXCR4 antagonists are also used as HPC-mobilizing agents, for example AMD3100, and may be applicable in the present invention.

In yet some further embodiments, the patient treated by the method of the invention may be subjected to preceding treatment that may involve immunoabalation. Treatment of some types of medical conditions, such as cancers, autoimmune diseases and the like often involves an immunoablation to remove the patient's own immune system, for example, prior to transplant of a bone marrow or other tissue graft. Immunoablation can be accomplished by total body radiation or by high dose chemotherapy. Therefore, the term “immune ablation” or “immunoablation” refers to the destruction of a patient immune resistance for a medical purpose.

Still further, in some embodiments, the patient treated by the method of the invention may be subjected to preceding treatment with steroids. In some embodiments, such treatment may be Steroid administration may be also used to inhibit the immune response. In pharmacologic (supraphysiologic) doses, steroids such as glucocorticoids are used to suppress various allergic, inflammatory, and autoimmune disorders.

In some specific embodiments, the nucleic acid cassette administered to the treated subject, may be flanked on both the 5′ and 3′ ends thereof by RSS.

In more specific embodiments, such RSS may be at least one of 12 RSS, 23 RSS and 22 RSS. In some particular and non-limiting embodiments, RSS applicable in the present invention may by any of the RSSs as denoted by nucleic acid sequence of SEQ ID NO. 36, 37, 38, 39, 40 and 41.

In one embodiments, the RSS used by the methods and cassettes of the invention may be naturally occurring, selected by a screen, rationally optimized, selected based on compatibility or incompatibility with other RSSs.

In yet some further embodiments, the cassette administered by the method of the invention may further comprise at least one genetic element. More specifically, in so embodiments, the genetic element may be at least one of: an IRES, a 2A peptide coding sequence, a promoter or any functional fragments thereof (e.g., a minimal promoter), a splice donor, a splice acceptor, a degron, a 3 frame stop, a protein stabilizing sequence, a signal peptide, a stop codon, a polyadenylation site, a transcription enhancer, a switch region, an mRNA stabilizing sequence and a protein stabilizing sequence.

In some particular embodiments, the cassette may comprise two or more nucleic acid sequences of interest separated by at least one genetic element, said genetic element may be at least one of: an IRES, a 2A peptide coding sequence and a promoter.

It should be noted that the nucleic acid sequence of interest inserted into a specific locus by the method of the invention may be a protein coding nucleic acid sequence or a non-coding sequence.

In some specific embodiments, such nucleic acid sequence of interest may be a protein coding nucleic acid sequence. More specifically, such gene may encode at least one of: a therapeutic protein or peptide, a prophylactic protein, a tolerizing protein, an immunizing protein, a toxic protein, an inducible suicide protein, a marker protein, an imaging protein and a fusion or chimeric protein. It should be understood that any of the proteins encoded by the nucleic acid sequence of interest described herein before in connection with other aspects of the invention are applicable for this aspect as well.

In yet some alternative or additional embodiments, the nucleic acid sequence of interest may encode at least one small non-coding RNA molecule, for example, miRNA, siRNA, shRNA and the like. In yet some further embodiments, the small non-coding RNA molecule may be specifically directed against a TCR chain, a BCR chain, an immune checkpoint gene, an apoptotic gene.

In certain embodiments, the nucleic acid sequence of interest may encode at least one of a receptor and an antibody or any fragment/s or chimera/s thereof. In more specific embodiments, the nucleic acid sequence of interest may encode at least one receptor, specifically, a TCR, CAR and a BCR or any combination thereof. In some embodiments, the nucleic acid sequence of interest may encode a receptor that may be a naturally occurring receptor, receptor selected by a screen or rationally designed receptor.

In yet some further embodiments, the nucleic acid sequence of interest may encode at least one antibody. More specifically, such antibody may be any one of full length antibody, antibody fragment, scFv, bi-specific antibody, tri-specific antibody, BiTE and V-NAR.

In some embodiments, the nucleic acid sequence of interest may encode an antibody that may be a naturally occurring antibody, antibody or fragments thereof selected by a screen or rationally designed antibody.

Still further, in some particular embodiments, the invention provides a method for engineering of inducible B-cells. More specifically, the nucleic acid cassette administered to the treated subject by the methods of the invention may comprise a nucleic acid sequence encoding a secreted antibody directed against and specific for an autoantigen and additionally, a nucleic acid sequence encoding a BCR directed against and specific for a foreign antigen. It should be noted that both nucleic acid sequences may be separated by at least one genetic element, for example, an IRES or a 2A peptide coding sequence. In certain embodiments, the method further comprises the step of administration of an effective amount of the foreign antigen (e.g., Polio vaccine for inducing B cell differentiation to plasma cells secreting Abs specific for the autoantigen) to the treated subject thereby inducing differentiation of the B cells into antibody secreting plasma cells.

In certain embodiments, the target genomic locus targeted by the cassette administered by the method of the invention may be at least one of Immunoglobulin heavy chain locus, Immunoglobulin κ chain locus, Immunoglobulin A chain locus, TCRβ chain locus, TCRα chain locus, TCRγ chain and the TCRδ chain locus.

In some specific embodiments, for in vivo targeted insertion of the nucleic acid sequence of interest, the subject may be administered with a nucleic acid vector comprising the cassette. In more specific embodiments, such vector may be any one of a viral vector, a non-viral vector and a naked DNA vector.

In more specific embodiments, either for in vivo or for ex vivo targeted insertion, a viral vector may be used. In yet more specific embodiments, such viral vector may be any one of rAAV, ssAAV, scAAV, SV40 vector, Adeno virus vector, helper-dependent Adeno viral vector, retroviral vector and lentiviral vector.

In yet some alternative embodiments, the vector used by the method of the invention may be a non-viral vector, specifically, any one of plasmid, minicircle and linear DNA.

In yet some further embodiments, an appropriate vector for the method of the invention may be naked DNA vector, specifically, any one of plasmid, minicircle and linear DNA.

Still further, in some embodiments for in vivo targeted insertion of the nucleic acid sequence of interest, the nucleic acid cassette or any vector comprising the same used by the method of the invention, may be administered by at least one of systemic injection, intrathymic injection, bone marrow injection splenic injection and injection to lymph nodes.

More specifically, in some embodiments where engineered T cells are desired, the method of the invention intrathymic injection. Intrathymic injection is a procedure used in several T cell-associated immunological contexts to deliver cells or other substances directly into the thymus. In the context of the present invention, the nucleic acid cassette of the invention or any vector or composition thereof may be injected into the thymus, thereby specifically targeting differentiating T cells. In some embodiments, the nucleic acid cassette or the vector may be injected via intrathymic injection, in such case the target cells may be thymocytes, specifically, thymocytes of the DN1, DN2, DN3, DN4, DP and SP subsets).

In case of bone marrow injection, the target cells may be HSPCs and in case of systemic injection, the target cells may be mobilized HSPCs (where the patient is subjected to a preceding treatment of immobilization). It should be noted that both, the bone marrow injection as well as systemic injection (e.g., intravenous IV), may be specifically suitable for targeting B cells by the nucleic acid cassette of the invention. It is to be understood that other localized injection are also suitable, for example intra-lymph node injection or intra-spleen injection, and may be used to deliver a vector to the lymph node and the spleen, respectively. More specifically, in some embodiments, the subcutaneous route (SC) may be generally considered to be most appropriate for targeting to the lymph nodes. Conventional SC formulations are such as Aqueous solutions, Oily solutions, Suspensions and Simple emulsions. Specific technologies associated with SC delivery are via Modified release SC formulations such as Biodegradable in situ implants, Biodegradable microspheres, Osmotically controlled implants, Liposomes, Lipid nanoparticles. Relevant commercially available products may include but are not limited to Alzamer® Depot™, DUROS®, Stealth1 (ALZA Corporation), Atrigel® (Atrix Laboratories), SABER® (Durect Corp), ProLease (Alkermes Inc), DepoFoam® (SkyePharma Inc), SupraVail™ (Phares Drug Delivery AG).

In some embodiments, the nucleic acid sequence of interest may be inserted into a target genomic locus in cells of the treated subject that are RAG expressing, either naturally or stably RAG expressing, inducibly RAG expressing and transfected with RAG expression vectors. In some embodiments, RAG expressing cells may be cells that undergo VDJ recombination. However, it should be understood that this term may further encompasses cells that already underwent VDJ recombination but still display RAG expression.

In yet some further embodiments, where the targeted insertion of the nucleic acid sequence of interest is performed ex vivo, the treated subject may be administered with at least one cell transduced or transfected with the nucleic acid cassette.

In yet some specific embodiments, administration of the cells transfected or transformed by the nucleic acid cassette of the invention may be performed by adoptive transfer or implantation. In yet some further embodiments, the cassette may be introduced to the target cells by transfection, electroporation or in case of viral vectors, by transduction. Any suitable method known to the skilled artisan may be applicable herein.

As noted above, in more specific embodiments, the cell administered by the method of the invention may be any one of a lymphocyte progenitor, ex vivo differentiating lymphocytes, HSPCs and iPSCs.

Still further, in some embodiments, the cells may be of an autologous or allogeneic source.

In some embodiments, the cells transduced or transfected with the nucleic acid cassette provided by the invention may be cells of an autologous source. The term “autologous” when relating to the source of cells, refers to cells derived or transferred from the same subject that is to be treated by the method of the invention.

In yet some further embodiments, the cells transduced or transfected with the nucleic acid cassette used by the methods of the invention may be cells of an allogenic source.

The term “allogenic” when relating to the source of cells, refers to cells derived or transferred from a different subject, referred to herein as a donor, of the same species.

It should be noted that in certain embodiments, where adoptive transfer of cells (either of autologous or allogeneic source) that comprise the cassette of the invention is applied, the method of the invention may further comprise the preceding step of immunoabalation of the subject, as described herein before.

The methods of the invention may be in some embodiments thereof, specifically suitable for disorders associated with the immune system.

Thus, in some specific embodiments, the subject treated by the method of the invention may be a subject suffering of an immune-related disorder. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., viral, bacterial or fungal infections), inflammatory disease, autoimmune disorders, metabolic disorders and a proliferative disorders, specifically, cancer. In some specific embodiments wherein the immune-related disorder or condition may be a primary or a secondary immunodeficiency.

In some specific embodiments, the methods of the invention may be used for treating proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors.

Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. It should be appreciated that for treating cancer, the cassette of the invention or any compositions or methods thereof may facilitate V(D)J targeted insertion or antibody or receptor as described herein before, that are specifically directed at TAAs. It should be understood that the invention thus encompasses the treatment of any of the malignancies described in this context, specifically any malignancies described in connection with associated TAAs as described herein before in connection with other aspects of the invention

In yet some further embodiments, and of particular relevance are patients' populations diagnosed with one of autoimmune disorders, also referred to as disorders of immune tolerance, when the immune system fails to properly distinguish between self and non-self-antigens.

Thus, according to some embodiments, the method of the invention may be used for the treatment of a patient suffering from any autoimmune disorder. In some specific embodiments, the methods of the invention may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, systemic lupus erythematosus (SLE), Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In yet some other embodiments, the methods of the invention may be also applicable for treating a subject suffering from an infectious disease. More specifically, such infectious disease may be any pathological disorder caused by a pathogen.

As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the invention may be applicable in boosting the immune response against a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, rubella virus, hepatitis C (HBC), arboviruses, rabies virus, influenza viruses A and B, measles virus, mumps virus, human deficiency virus (HIV), HTLV I and II and Zika virus.

In some further embodiments, the methods of the invention may be applicable for immune-related disorder or condition that may be a pathologic condition caused by at least one pathogen. It should be appreciated that an infectious disease as used herein also encompasses any infectious disease caused by a pathogenic agent, specifically, a pathogen. Pathogenic agents include prokaryotic microorganisms, lower eukaryotic microorganisms, complex eukaryotic organisms, viruses, fungi, prions, parasites, yeasts, toxins and venoms. In yet some other specific embodiments, the methods and composition of the invention may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulimim, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous, and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present invention provides compositions and methods for use in the treatment, prevention, amelioration or delay the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal.

It should be appreciated that an infectious disease as used herein also encompasses any pathologic condition caused by toxins and venoms.

Thus, the methods of the invention may offer a promising therapeutic modality for a variety of innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immunosurveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune-deficiencies are genetic disorders; the majority is diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, such secondary immunodeficiency may be caused by at least one of chemotherapy, radiotherapy, biological therapy, bone marrow transplantation, gene therapy, adoptive cell transfer or any combinations thereof.

As described herein above, the invention provides in some aspects thereof therapeutic and prophylactic methods.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention.

As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

As described above, the nucleic acid cassette of the invention may be administered by the methods of the invention either ex vivo, by introduction thereof into cells that are being transplanted or transferred to the treated subject, or alternatively in vivo, where the cassette or any vector or composition thereof are directly administered to the subject.

It should be therefore understood that the number of administrations of treatment to a subject may vary. Introducing the genetically modified cells that comprise the cassette of the invention, into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated. In other aspects of the invention as discussed above, the nucleic acid cassette for V(D)J targeted insertion of a nucleic acid sequence of interest is employed to modify cellular DNA in vivo. In these in vivo embodiments, the nucleic acid cassette of the invention may be administered directly to the individual. The nucleic acid cassette may be administered by any of a number of well-known methods in the art for the administration of nucleic acids to a subject. The nucleic acid cassette can be incorporated into a variety of formulations. More particularly, nucleic acid cassette of the present invention can be formulated into pharmaceutical compositions by combination with appropriate pharmaceutically acceptable carriers or diluents. Appropriate pharmaceutical composition applicable for this aspect will be described in more detail herein after.

In yet a further aspect, the invention relates to a nucleic acid cassette. More specifically, the cassette of the invention may comprise the nucleic acid sequence of interest and at least one RSS. In some embodiments, the cassette provided by the invention may be applicable for targeted insertion of at least one nucleic acid sequence of interest into a target genomic locus. It should be further noted that the insertion of the nucleic acid sequence of interest into the target genomic locus may be facilitated, mediated and/or performed by RAG-catalyzed recombination between at least one genomic RSS flanking the target genomic locus and at least one RSS comprised within the nucleic acid cassette.

It should be appreciated that the nucleic acid cassette of the invention, as being dependent on RAG activity, may be also referred to herein as the “V(D)J targeting cassette” or “V(D)J targeting nucleic acid cassette”.

In some specific embodiments, the nucleic acid cassette may be flanked on both the 5′ and 3′ ends thereof by RSS.

In yet some further specific embodiments, the RSS may be at least one of 12 RSS, 23 RSS and 22 RSS. In some particular and non-limiting embodiments, RSS applicable in the present invention may by any of the RSSs as denoted by nucleic acid sequence of SEQ ID NO. 36, 37, 38, 39, 40 and 41.

Therefore, in some embodiments the cassettes of the invention may be flanked either by “same” RSSs (two 12 RSSs or two 23 RSSs), or by an incompatible pair of a 12 RSS and a 23 RSS.

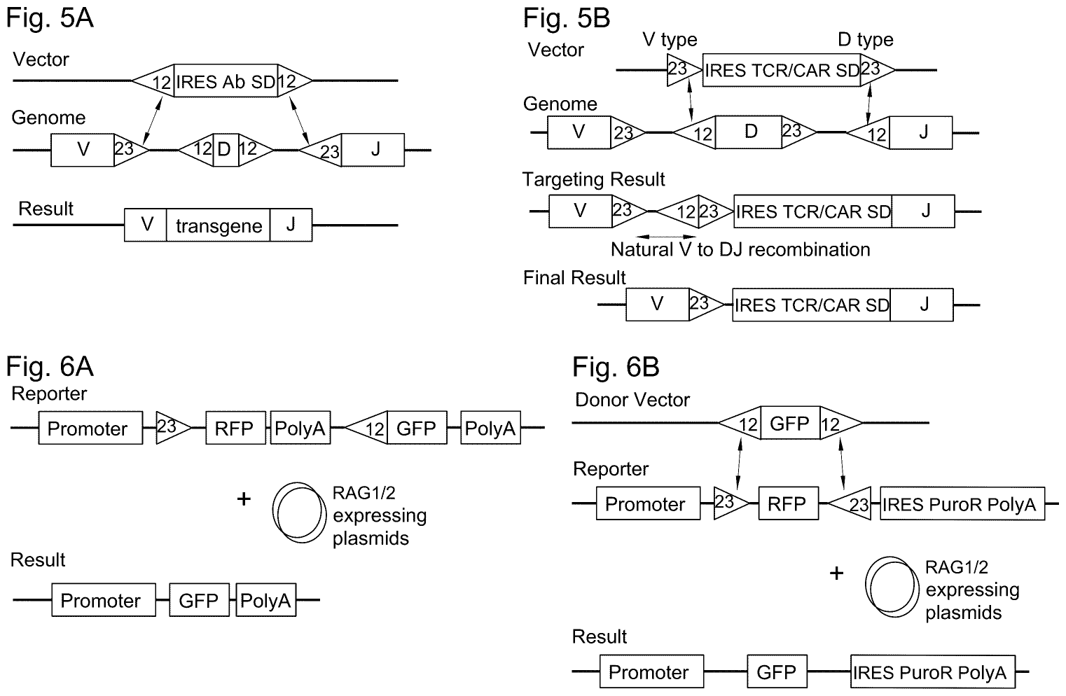

In some particular embodiments specifically suitable for VDJ targeting in the B lineage, the cassette of the invention may be flanked by two “outward facing” 12 RSSs to integrate between a V and a J segment of the heavy chain (as also illustrated by FIG. 5A). In yet some further alternative embodiments, a suitable configuration for VDJ targeting in the T lineage, may be a cassette flanked by two “downstream facing” 23 RSSs to integrate between a D and a J segment of a β chain (as also illustrated by FIG. 5B).

In some embodiments, the RSS used by the methods and cassettes of the invention may be naturally occurring, selected by a screen, rationally optimized, selected based on compatibility or incompatibility with other RSSs.

In certain embodiments, the cassette of the invention may further comprise at least one genetic element. Specifically, such genetic element may be at least one of: an internal ribosome entry site (IRES), a 2A peptide coding sequence, a promoter and any fragments thereof, a splice donor, a splice acceptor, a degron, a 3 frame stop, a protein stabilizing sequence, a signal peptide, a stop codon, a polyadenylation site, a transcription enhancer, a switch region, an mRNA stabilizing sequence and a protein stabilizing sequence.

As noted above, several genetic elements may be included in the cassette of the invention. In some specific embodiments, at least one SD may be included. More specifically, for VDJ targeting in the B cell lineage, a heavy chain constant region may not be encoded by the cassette of the invention, but instead a downstream splice donor (SD) may be included therein thereby allowing the utilization of endogenous constant region. In some embodiments, such cassette may encode an Ab light chain (V_{L}J_{L}C_{L}) and a variable region of a heavy chain (V_{H}D_{H}J_{H}) separated by a 2A peptide, more specifically, such cassette may include V_{L}J_{L}C_{L}-2A-V_{H}D_{H}J_{H}-SD. In some specific embodiments, at least one SA may be included. In some embodiments, such cassette may encode an Ab light chain (V_{L}J_{L}C_{L}) and a variable region of a heavy chain (V_{H}D_{H}J_{H}) separated by a 2A peptide, more specifically, such cassette may include SA-V_{L}J_{L}C_{L}-2A-V_{H}D_{H}J_{H}-SD.

In yet some further embodiments, a minimal promoter (mP) may be included in the cassette of the invention. In some embodiments, such cassette may encode an Ab light chain (V_{L}J_{L}C_{L}) and a variable region of a heavy chain (V_{H}D_{H}J_{H}) separated by a 2A peptide, more specifically, such cassette may include: mP-V_{L}J_{L}C_{L}-2A-V_{H}D_{H}J_{H}-SD.

In yet some further embodiments, for VDJ targeting in the T cell lineage, several different specific configurations for VDJ targeting may be used. More specifically, in some embodiments, when targeting a transgenic TCR cassette to the TCR β chain locus, a heavy chain constant region may not be encoded by the cassette of the invention but instead, the cassette may include a downstream SD to allow the utilization of endogenous constant region. In some embodiments, the cassette may encode a TCR α chain (VαJαCα) and a variable region of a β chain (VβDβJβ) separated by a 2A peptide, specifically, VαJαCα-2A-VβDβJβ-SD. Still further in some embodiments, when targeting a CAR cassette to the TCR β chain locus, the cassette of the invention may comprise a nucleic acid sequence encoding the full CAR gene, ending with a stop codon, and only then a SD. In some specific embodiments, at least one SA may be included. In some embodiments, such cassette may encode an TCR α chain (VαJαCα) and a variable region of a β chain (VβDβJβ) separated by a 2A peptide, specifically, such cassette may include SA-VαJαCα-2A-VβDβJβ-SD.

In yet some further embodiments, a minimal promoter (mP) may be included in the cassette of the invention. In some embodiments, such cassette may encode an TCR α chain (VαJαCα) and a variable region of a β chain (VβDβJβ) separated by a 2A peptide, specifically, such cassette may include: mP-VαJαCα-2A-VβDβJβ-SD.

In still some further specific embodiments, the cassette of the invention may comprise two or more nucleic acid sequences of interest separated by at least one genetic element. Such genetic element may be at least one of: an IRES, a 2A peptide coding sequence and a promoter. Thus, in some embodiments, the cassette of the invention may encode several peptides (e.g. two Ab chains) and these may be separated by 2A peptides. In yet some specific embodiment a preceding IRES and an intervening 2A peptide may be included in the cassette of the invention that encodes the heavy and the light chains, specifically, such cassette may comprise RSS-IRES-V_{L}J_{L}C_{L}-2A-V_{H}D_{H}J_{H}-SD-RSS. More specifically, an IRES sequence followed by a sequence encoding a light chain V, J and C segments. An 2A sequence separates between the light chain and heavy chain segments VDJ that are followed by a splice donor site that allow that allow specific splicing to include the endogenous C region.

In yet some further embodiments, the nucleic acid sequence of interest may be a protein coding nucleic acid sequence or a non-coding sequence.

In some specific embodiments, the nucleic acid sequence of interest may be a protein coding gene. More specifically, such protein may be at least one of: a therapeutic protein or peptide, a prophylactic protein, a tolerizing protein, an immunizing protein, a toxic protein, a suicide protein, specifically, an inducible suicide protein, a marker protein, an imaging protein and a fusion or chimeric protein.

In some specific and non-limiting embodiments, a suicide gene may be further comprised within the cassette of the invention. In yet some specific embodiments the invention provides a cassette comprising an inducible suicide gene for the specific elimination of targeted lymphocytes, specifically, in case the expression is found to be associated with adverse outcomes. In more particular embodiments, the cassette of the invention may further comprise an upstream caspase gene fused to an inducible dimerization domain (iCasp9). In certain embodiments, the cassette of the invention may comprise RSS-IRES-iCasp9-2A-V_{L}J_{L}C_{L}-2A-V_{H}D_{H}J_{H}-SD-RSS. More specifically, an IRES sequence followed by a sequence encoding caspase 9, followed by 2A sequence that separates between the caspase and a light chain V, J and C segments. An additional 2A sequence separates between the light chain and heavy chain segments VDJ that are followed by a splice donor site that allow that allow specific splicing to include the endogenous C region.

In yet some further embodiments, the nucleic acid sequence of interest may encode at least one of a small non-coding RNA molecule, specifically, miRNA, siRNA, shRNA and the like. In yet some further embodiments, the small non-coding RNA molecule may be specifically directed against a TCR chain, a BCR chain, an immune checkpoint gene, an apoptotic gene.

More specifically, in some embodiments, the cassette of the invention may target a full TCR gene (TCRα-2A-TCRβ) into the TCRβ chain locus, in such case, an miRNA directed against the TCRα chain, may be further added in the cassette to prevent cross-dimerization. In yet some further embodiments, miRNAs against the non-targeted allele may be used. Knockdown of the other allele is desired only if natural allelic exclusion is absent or incomplete, for example when introducing a CAR gene rather than a TCR and thus not providing all natural signals for allelic exclusion. Still further, in some embodiments, the cassette of the invention may further comprise nucleic acid sequence encoding miRNA against the immune checkpoint protein PD1 in T cells. In some further embodiments, the cassette of the invention may further comprise nucleic acid sequence encoding a miRNA against an apoptotic gene. In further embodiments, such miRNA may be used in conjunction with an inducible suicide gene as described above. Accordingly, in certain embodiments, the cassette of the invention may comprise RSS-IRES-iCasp9-2A-V_{L}J_{L}C_{L}-2A-V_{H}D_{H}J_{H}-SD-miRNA-RSS. More specifically, an IRES sequence followed by a sequence encoding caspase 9, followed by 2A sequence that separates between the caspase and a light chain V, J and C segments. An additional 2A sequence separates between the light chain and heavy chain segments VDJ that are followed by a splice donor site, and an additional sequence encoding miRNA. The cassette is flanked by an additional RSS sequence.

In yet some further embodiments, the nucleic acid sequence of interest comprised within the cassette of the invention may encode at least one of a receptor and an antibody or any fragment/s or chimera/s thereof.

In some specific embodiments, the nucleic acid sequence of interest of the cassette of the invention may encode at least one receptor. Specifically, any one of, or at least one of a TCR, CAR and a BCR.

In some embodiments, the nucleic acid sequence of interest may encode a receptor that may be a naturally occurring receptor, receptor selected by a screen or rationally designed receptor.

More specifically, for V(D)J targeting T cells, receptors targeted at specific antigens, may be used in the cassettes of the invention for targeted integration of several different receptors of clinical relevance. Thus, in some specific embodiments, the cassette of the invention may comprise a nucleic acid sequence encoding an anti CD19 CAR. Such cassette may be effective for treating B cell malignancies. In some specific embodiments, such CD19 CAR may comprise the amino acid sequence as denoted by SEQ ID NO. 10 or any derivatives thereof. In yet some further embodiments, the cassette of the invention may comprise a nucleic acid sequence encoding an anti-Tn-MUC1 CAR, which targets a highly cancer specific glycoform. In some specific embodiments, such Tn-MUC1 CAR may comprise the amino acid sequence as denoted by SEQ ID NO. 11 or any derivatives thereof. In some further embodiments, the cassette of the invention may comprise nucleic acid sequence encoding an NY-ESO-1-specific TCR construct. In some specific embodiments, such NY-ESO-1CAR may comprise the amino acid sequence as denoted by SEQ ID NO. 12 or any derivatives thereof.

In certain specific embodiments, the nucleic acid sequence of interest may encode at least one antibody. In more particular embodiments, such antibody may be any one of: full length antibody, antibody fragment, scFv, bi-specific antibody, tri-specific antibody, BiTE and V-NAR.

In some embodiments, the nucleic acid sequence of interest may encode an antibody that may be a naturally occurring antibody, antibody or fragments thereof selected by a screen or rationally designed antibody.

In more specific embodiments, for targeting B cells, specifically, differentiating B cells that express RAG, V(D)J cassette for targeted insertion of antibody sequences, specifically, antibodies of clinical relevance. More particularly, in some embodiments, the cassette of the invention may comprise a nucleic acid sequence encoding at least one anti-viral antibody, specifically, at least one anti-HIV antibody, more specifically, the 3BNC117 antibody. In some particular and non-limiting embodiments such 3BNC117 antibody may comprise the amino acid sequence as denoted by SEQ ID NO. 13 or any derivatives thereof. In yet some further embodiments, the cassette of the invention may comprise a nucleic acid sequence encoding at least one anti-TNFα antibody, specifically, an antibody effective in auto-immune diseases, more specifically, the Adalimumab (Humira™) antibody. In some particular and non-limiting embodiments such Adalimumab antibody may comprise the amino acid sequence as denoted by SEQ ID NO. 14 or any derivatives thereof. In yet some further embodiments, the cassette of the invention may comprise a nucleic acid sequence encoding at least one anti MUC1 glycoform antibody, specifically, the PankoMab-GEX. In some particular and non-limiting embodiments such PankoMab-GEX antibody may comprise a heavy chain variable region that comprises the amino acid sequence as denoted by SEQ ID NO. 15, and a light chain variable region comprising the amino acid sequence as denoted by SEQ ID NO. 16 or any derivatives thereof.

In yet some further specific embodiments, the nucleic acid cassette of the invention may comprise a nucleic acid encoding a secreted antibody directed against or specific for an autoantigen. In yet some further embodiments, said cassette may further comprise a nucleic acid sequence encoding a BCR directed against and/or specific for a foreign antigen.

A non-limiting example for such cassette that offers an inducible secretion of antibodies, is illustrated in FIG. 20. Such cassette may include RSS-IRES-scFv/BiTe (specific for an autoantigen)-IRES-BCR (specific for a foreign antigen)-RSS.

In some embodiments, the target genomic locus targeted by the cassette of the invention may be at least one of Immunoglobulin heavy chain locus, Immunoglobulin κ chain locus, Immunoglobulin λ chain locus, TCRβ chain locus, TCRα chain locus, TCRγ chain and the TCRδ chain locus.

In certain embodiments, the cassette of the invention may be comprised within a nucleic acid vector. More specifically, such vector may be any one of a viral vector, a non-viral vector and a naked DNA vector.

It should be appreciated that any of the nucleic acid cassettes described herein as specific and non-limiting embodiments, are encompassed by the invention and may be used in any of the methods described herein before and in any of the compositions described herein after.

The invention provides nucleic acid cassette and methods using the cassette. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence.

Still further, it should be understood that the invention encompasses as additional aspects thereof any vector or vehicle that comprise any of the cassettes described by the invention.

Still further, the invention also provides any cell, specifically a mammalian host cell that express the cassettes of the invention or any of the vectors or vehicles described by the invention. In some specific embodiments, such host cells may be immune-related cells, specifically lymphocytes, more specifically, lymphocytes either obtained in some embodiments from a healthy subject or in other embodiments, obtained from a subject suffering from an immune-related disorder. Specific embodiments relate to B, T and NK T lymphocytes that are transduced or transfected with the cassettes of the invention or any vector or vehicle thereof. It should be understood that any of the cells, vectors and subjects described by the invention also apply for these aspects as well.

In yet a further aspect, the invention relates to a pharmaceutical composition comprising an effective amount of at least one nucleic acid cassette, or any vector or cell comprising the cassette of the invention. More specifically, the cassette of the composition of the invention may comprise the nucleic acid sequence of interest and at least one RSS. In some embodiments, the cassettes provided by the compositions of the invention may be applicable for targeted insertion of at least one nucleic acid sequence of interest into a target genomic locus. It should be noted that the insertion of the nucleic acid sequence of interest into the target genomic locus may be facilitated, mediated and/or performed by RAG-catalyzed recombination between at least one genomic RSS flanking the target genomic locus and at least one RSS comprised within said nucleic acid cassette. In some optional embodiments, the compositions of the invention may further comprise at least one of pharmaceutically acceptable carrier/s, diluent/s, excipient/s and additive/s.

The compositions of the invention may comprise an effective amount of the cassette of the invention or of any vector thereof or of any cell comprising the same. The term “effective amount” relates to the amount of an active agent present in a composition, specifically, the cassette of the invention as described herein that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of a the V(D)J targeting cassette of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The pharmaceutical compositions of the invention can be administered and dosed by the methods of the invention, in accordance with good medical practice, systemically, for example by parenteral, e.g. intrathymic, into the bone marrow and intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ (thymus, bone marrow, spleen, lymph nodes), etc. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprises at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the V(D)J targeting cassette of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question.

Still further, pharmaceutical preparations are compositions that include one or more targeting cassette present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the V(D)J targeting cassette can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

Still further, the composition/s of the invention and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the combination/s, composition/s and kit/s of the invention or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

Still further, it should be understood, that the invention further encompasses any cell that comprise the nucleic acid cassette of the invention. In more particular embodiments, the cell of the invention may be transformed or transfected with at least one nucleic acid cassette according to the invention that enables the targeted insertion of a nucleic acid sequence of interest into a target locus within the cell. It should be noted that any of the cells discussed above may applicable for this aspect, specifically, any lymphocyte progenitor, ex vivo differentiating lymphocytes, HSPCs and iPSCs. It should be appreciated that these cells may be used for the therapeutic and prophylactic methods of the invention and may therefore include in some embodiments thereof cells of an autologous or allogeneic source.

As used herein, a cell has been “transformed” or “transfected” by exogenous or heterologous DNA, e.g. the V(D)J cassette of the invention, when such DNA has been introduced inside the cell. The transforming DNA may be integrated (covalently linked) into the genome of the cell. With respect to the present invention, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

In yet a further aspect, the invention provide an effective amount of at least one nucleic acid cassette or any composition thereof for use in a method for targeted insertion of at least one nucleic acid sequence of interest into a target genomic locus in a mammalian cell. More specifically, the cassette of the invention may comprise the nucleic acid sequence of interest and at least one RSS. It should be further noted that the insertion of the nucleic acid sequence of interest into the target genomic locus may be facilitated, mediated and/or performed by RAG-catalyzed recombination between at least one genomic RSS flanking the target genomic locus and at least one RSS comprised within the nucleic acid cassette. The invention further provides an effective amount of at least one of the cassettes described herein, any compositions or vehicles thereof or any cell expressing the cassette of the invention for use in a method for targeted insertion of at least one nucleic acid sequence of interest into a target genomic locus of a cell in a mammalian subject.

In a further aspect of the invention, the invention provide an effective amount of at least one nucleic acid cassette or any composition thereof or vehicles thereof or any cell expressing the cassette of the invention for use in a method for treating, preventing, ameliorating, inhibiting or delaying the onset of a pathologic disorder in a mammalian subject. More specifically, the cassette of the invention may comprise the nucleic acid sequence of interest and at least one RSS. The cassette administered to the treated subject targets insertion of the at least one nucleic acid sequence of interest comprised therein into a target genomic locus in a the treated subject. It should be further noted that the insertion of the nucleic acid sequence of interest into the target genomic locus in the treated subject may be facilitated, mediated and/or performed by RAG-catalyzed recombination between at least one genomic RSS flanking the target genomic locus and at least one RSS comprised within the nucleic acid cassette.

In some specific embodiments the nucleic acid cassette or any compositions or vectors thereof for use in accordance with the invention, may be flanked on both the 5′ and 3′ ends thereof by RSS.

In more specific embodiments, the RSS comprised within the cassette for use in accordance with the invention may be at least one of 12 RSS, 23 RSS and 22 RSS. In some particular and non-limiting embodiments, RSS applicable in the present invention may by any of the RSSs as denoted by nucleic acid sequence of SEQ ID NO. 36, 37, 38, 39, 40 and 41.

In some embodiments, the RSS of the cassettes for use in accordance with the invention may be naturally occurring, selected by a screen, rationally optimized, selected based on compatibility or incompatibility with other RSSs.

In yet some further specific embodiments, the nucleic acid cassette for use in accordance with the invention may further comprise at least one genetic element, said genetic element is at least one of: an IRES, a 2A peptide coding sequence, a promoter or any functional fragment thereof, a splice donor, a splice acceptor, a degron, a 3 frame stop, a protein stabilizing sequence, a signal peptide, a stop codon, a polyadenylation site, a transcription enhancer, a switch region, an mRNA stabilizing sequence and a protein stabilizing sequence.

In some specific embodiments, the cassette for use in accordance with the invention may comprise two or more nucleic acid sequences of interest separated by at least one genetic element. In more specific embodiments, such genetic element may be at least one of an IRES, a 2A peptide coding sequence and a promoter.

In certain embodiments, the nucleic acid sequence of interest comprised within the cassette for use in accordance with the invention may be a protein coding nucleic acid sequence or a non-coding sequence.

In some specific embodiments, where the nucleic acid sequence of interest is a protein coding gene, such protein may be at least one of: a therapeutic protein or peptide, a prophylactic protein, a tolerizing protein, an immunizing protein, a toxic protein, an inducible suicide protein, a marker protein, an imaging protein and a fusion or chimeric protein.

In yet some further alternative embodiments, the nucleic acid sequence of interest may encode at least one small non-coding RNA molecule. More specifically, such one small non-coding RNA molecule may be for example, miRNA, siRNA, shRNA or any of the one small non-coding RNA molecules described by the invention herein before. In some further embodiments, the small non-coding RNA molecule may be specifically directed against a TCR chain, a BCR chain, an immune checkpoint gene or an apoptotic gene.

In more specific embodiments, the nucleic acid sequence of interest of the cassette for use in accordance with the invention may encode at least one of a receptor and an antibody or any fragment/s or chimera/s thereof.

In some particular embodiments, the nucleic acid sequence of interest may encode at least one receptor. More specifically, such receptor may be any one of: a TCR, CAR and a BCR.

In some embodiments, the nucleic acid sequence of interest may encode a receptor that may be a naturally occurring receptor, receptor selected by a screen or rationally designed receptor.

In yet some further particular embodiments, the nucleic acid sequence of interest may encode at least one antibody. More specifically, such antibody may be any one of full length antibody, antibody fragment, scFv, bi-specific antibody, tri-specific antibody, BiTE and V-NAR.

In some embodiments, the nucleic acid sequence of interest may encode an antibody that may be a naturally occurring antibody, antibody or fragments thereof selected by a screen or rationally designed antibody.

In some embodiments, the nucleic acid cassette for use in accordance with the invention may comprise a nucleic acid sequence encoding a secreted antibody directed against and specific for an autoantigen. This cassette may further comprise a nucleic acid sequence encoding a BCR directed against and specific for a foreign antigen. It should be noted that in certain embodiments, these two nucleic acid sequences are separated by at least one genetic element, for example, an IRES, a 2A peptide coding sequence and a promoter.

In certain embodiments, the target genomic locus for the targeted insertion of the nucleic acid sequence of interest comprised within the cassette for use in accordance with the invention may be at least one of Immunoglobulin heavy chain locus, Immunoglobulin κ chain locus, Immunoglobulin λ chain locus, TCRβ chain locus, TCRα chain locus, TCRγ chain and the TCRδ chain locus.

It should be appreciated that in certain embodiments, the nucleic acid cassette for use in accordance with the invention may be comprised within a nucleic acid vector. More specifically, such vector may be any one of a viral vector, a non-viral vector and a naked DNA vector.

In more specific embodiments, the vector may be a viral vector. Specifically, suitable viral vectors may be any one of rAAV, ssAAV, scAAV, SV40 vector, Adeno virus vector, helper-dependent Adeno viral vector, retroviral vector and lentiviral vector.

In yet some further embodiments, the cassette for use in accordance with the invention may be comprised within a non-viral vector. Such vector may be any one of plasmid, minicircle and linear DNA.

In yet some further embodiments, the vector may be a naked DNA vector, specifically, any one of plasmid, minicircle and linear DNA.

It should be noted that in certain embodiments, the cassette for use in accordance with the invention, leads to targeted insertion of the nucleic acid sequence of interest into a target locus in a cell of said mammalian subject. It should be noted that the targeted insertion may be mediated and facilitated by RAG complex. Thus, in some specific embodiments, the targeted cell/s of the subject may naturally express RAG. However, it should be noted that the invention further encompasses in some embodiments thereof, the option that the target cells of the administered subject may be either stably RAG expressing or inducibly RAG expressing. Such cells in some embodiments may be transfected with RAG expression vectors. As indicated herein before, in some embodiments, RAG expressing cells may be cells that undergo VDJ recombination. In yet some further embodiments, RAG expressing cell may further encompass cells that already underwent VDJ recombination but still express RAG.

In certain embodiments, the cell/s of the administered subject targeted by the cassette for use in accordance with the invention may be any one of thymocytes, HSPCs and mobilized HSPCs.

In yet some specific embodiments, the cells targeted by the cassette for use in accordance with the invention may be thymocytes of any subsect. More specifically, such cells may be thymocytes of any one of the DN1, DN2, DN3, DN4, DP and SP subsets, as described herein above. In yet some further embodiments, the cells targeted by the cassette for use in accordance with the invention may be splenocytes of any subsect, or any lymphocytes obtained or present in lymph nodes and bone marrow.

The uses described herein for the cassette or any cells, vehicles or compositions thereof may be applied in some embodiments to any disease, disorder, or natural cellular process that would benefit from modulating cell activity by integrating a gene of interest.

In some specific embodiments the uses described herein for the cassette or any cells, vehicles or compositions thereof may be applied for treating a subject in need. In more particular and non-limiting embodiments, such subject may be a subject suffering of an immune-related disorder. It should be noted that any of the immune related disorders described herein before in connection with other aspects of the invention are also applicable in the present aspects.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to +10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Materials and Reagents

### Reagents:

Imatinib: Gleevec® (STI-571, C₂₉H₃₁N₇O).

### Plasmids and Viral Vectors:

pGEMT (cat #A3600, by Promega)

Ad5-serotype-6 helper-free plasmid: pDGM6 obtained from the D.W. Russell lab at University of Washington.

### Cell Lines:

Jurkat cells ATCC® TIB-152™

V-Abl immortalized murine Pro B cells obtained from the L. Deriano lab at the Pasteur institute. HEK293-T-ATCC® CRL-3216™.

### Kits:

Quick-RNA™ microprep kit (Zymo Research).

Takara “All Serotype” purification kit (Cat #6666).

Quick-RNA™

### Animals and Animal Models:

C57BL/6 mice obtained from the Jackson labs.

### Experimental Procedures

Transfection with GFPi-Redless Plasmid:

V-Abl immortalized murine Pro B cells were transfected by electroporation (1600V, 20 ms, 1 pulse) with the GFPi-redless plasmid containing a 23-consensus RSS and a 12-consensus RSS. Cells recuperated for 4 hours, then given 3 μM Imatinib (Im) for induction of differentiation.

Integration by VDJ Targeting of a Gene Coding for an Antibody into the IgK Locus in Inducibly Differentiating B Cell Progenitors:

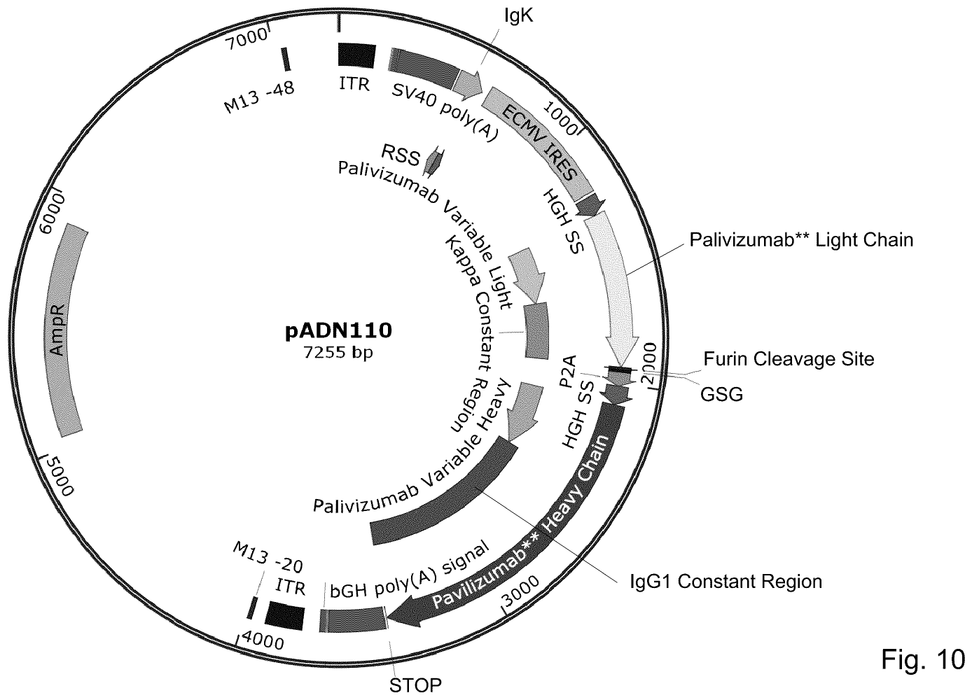

Immortalized and inducible differentiating Pro-B cells (Lescale et al. 2017 Mech Ageing Dev. 2017 July; 165 (Pt A): 3-9) were cultured at 37° C. with 5% CO₂. 10⁶ cells were taken, washed with PBS twice (Biological Industries) and then mixed with an amount of 2.3 μg Donor DNA (i.e. the plasmid pADN110, see illustrated map in FIG. 10). Neon™ Electroporation (Invitrogen) was performed using 10 μL tips (Invitrogen) at 1600v, 20 ms, 1 pulse. Neon™ Buffer R was replaced with Opti-MEM™ (Life Technologies) and Neon™ buffer E1 with Opti-MEM™ 10% Glycerol. Cells post electroporation were plated on RPMI 1640 (Biological Industries) supplemented with 10% FBS (Sigma) and 50 μM B-Mercaptoethanol. PBS was supplemented to media 24 h post electroporation. Imatinib was supplemented to media 24 h post electroporation to a concentration of 3 μM.

A volume of 1.5 ml was taken from culture for RNA extraction 3 days post electroporation, using Quick-RNA™ microprep kit (Zymo Research) according to the manufacturer's protocol. 2 μg RNA were taken for Reverse transcriptase reaction using oligo dT(15) primer with RevertAid Reverse transcriptase (ThermoFisher-Merck) according to manufacturer's protocol. PCR reaction on the cDNA template was done using 2×taq PCR MasterMix (Tiangen®) according to manufacturer's protocol. Gel electrophoresis was performed on a 1.7% Agarose gel supplemented with Ethidium Bromide. The following primers were used:

ECMV IRES R1 as denoted by SEQ ID NO: 19
5'-CATATAGACAAACGCACACCGGC-3'

IgKVm-rec-F1 as denoted by SEQ ID NO: 20

5′-GTCCCTGCCAGGTTYAGTGGCAGTGGRTCWRGGAC-3′. It should be noted that in accordance with IUPAC nomenclature, “Y” may be C or T, “R” may be a or G and “W” may be A or T.

### Extraction of Primary Mouse Bone Marrow and Enrichment for IL7-Receptor-Positive Progenitor Cells:

Two female C57Bl/6 mice, 8-12 weeks old, were used for each preparation of IL7-receptor-enriched bone marrow cells (IL7R⁺ cells). Hind and back longitude bones were flushed with RPMI medium containing 10% FBS, 100 U/ml Penicillin, 100 μg/ml Streptomycin and 2 mM EDTA. Clumps were disaggregated with a pipette and passed through a 40 μl cell strainer. No RBC lysis was performed. Pelleted cells were washed once with PBS (without C⁺⁺ and without Mg⁺⁺) supplemented with 10% FBS (PBS-FBS) and then re-suspended in 200 μl PBS-FBS, per mouse. PE-conjugated anti mouse IL7R (cat #130-102-551 by Miltenyi) was added at: 10 μl/mouse/200 μl PBS-FBS. Binding was performed at 4-6° C. for 30 min. Cell suspension was subsequently washed twice with PBS-FBS and re-suspended in 80 μl PBS-FBS per mouse. Magnetic nano-beads conjugated to a secondary antibody directed against the PE moiety of the primary antibody (cat #130-048-801 by Miltenyi) were added at: 20 μl/mouse/80 μl PBS-FBS. Binding was performed at 4-6° C. for 30 min. Cell-bead conjugates were washed once with PBS-FBS and separated on a magnetic column (cat #130-042-401 by Miltenyi). Typically, 2-4 million IL7R+ enriched cells per mouse were obtained. Cells were subsequently transduced, as detailed below, and cultured for in vitro differentiation in MEM-alpha medium, supplemented with 20% heat inactivated FBS, 100 U/ml Penicillin and 100 μg/ml Streptomycin.

### Flow Cytometry:

Staining was performed on non-fixed cells, in azide containing buffer, to prevent capping and internalization of expressed cell surface receptors.

The employed antibodies are as follows:

| Antibody: | Source | Cat# |
|---|---|---|
| Mouse IL7 receptor-PE | Miltenyi | 130-102-551 |
| Mouse IgK-Vio Blue | Miltenyi | 130-105-860 |
| Mouse IgM-APC | BioLegend | 406509 |
| Mouse Isotype control-APC | BioLegend | 400511 |
| Mouse CD19-FITC | BioLegend | 115505 |

Acquisition was performed on the “Attune NxT” flow cytometer and data analysis was performed with the TAU site-licensed Kaluza software.

Construction of a Minimal Promoter Trans-Recombination Donor Vector: dsAAV-360A:

A VDJ-targeting vector coding for a segment of a human IgK variable region, specifically, the Vκ 3-15 segment, and further coding for an HA tag was constructed. The human IgK variable region includes the natural V promoter, which is a “minimal promoter” i.e. it can only drive transcription when adjacent to an enhancer. Therefore, transcription occurs only after desired integration into the mouse VDJ loci (which are adjacent to a mouse enhancer at the Igκ/λ or IgH loci). The nucleic acid sequence of the above segment is as denoted by SEQ ID NO: 25.

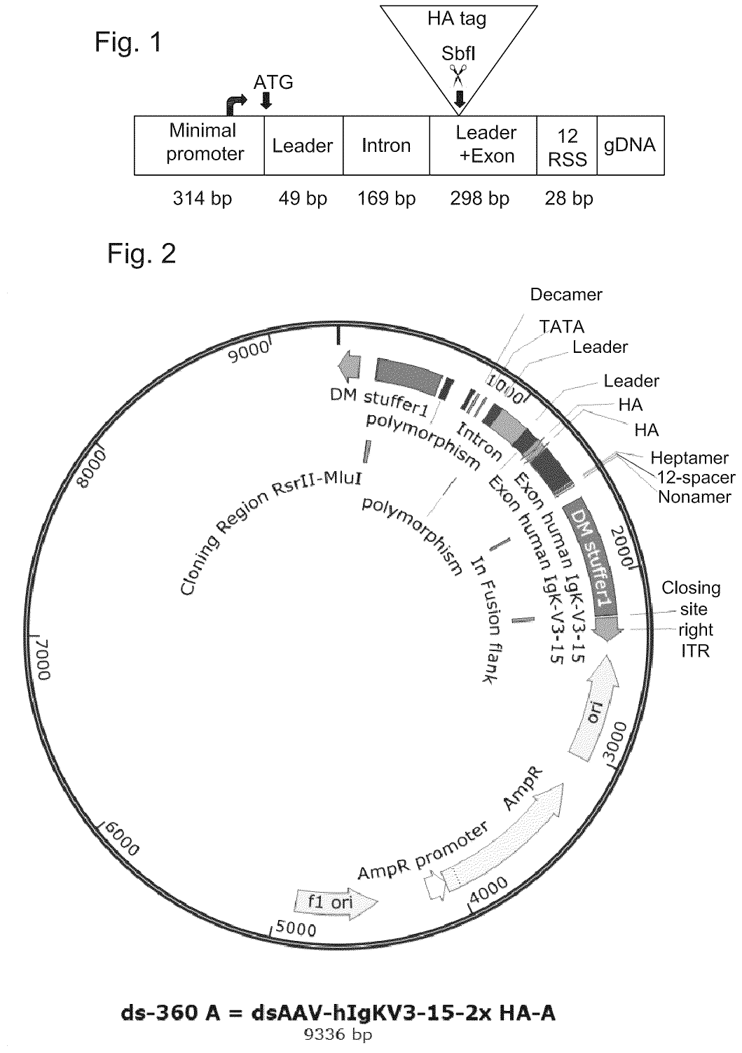

A natural human germline gene encoding for the IgKappa-V3-15 was PCR-amplified. The amplified region spanned 929 bases and included 314 bp of the minimal promoter that comprises the nucleic acid sequence as denoted by SEQ ID NO. 51 (upstream of the Leader ATG) and 71 bp downstream on the RSS (12-spacer). The gene was cloned into Bluescript SK−, and 2 tandem in-frame repeats of the HA tag (mouse codon optimized) were inserted into the natural SbfI site of the human IgKV3-15 gene (see schematic representation of the construct in FIG. 1). The HA-tagged gene was then cloned into a double strand AAV vector, which was pre-cloned to contain flanking Drosophila melanogaster (DM) stuffer sequences, to support optimal vector replication and packaging efficiency. The complete map of the dsAAV vector is illustrated in FIG. 2.

AAV Serotype 6 Preparation of dsAAV-360A Vector:

Vector stock was prepared by transfecting HEK293-T cells with the vector plasmid: dsAAV360A, alongside the Ad5-serotype-6 helper-free plasmid: pDGM6.

Purification was performed by using the Takara “All Serotype” purification kit (Cat #6666), according to the manufacturer's protocol.

dsAAV-360A Vector Transduction of IL7R+ Enriched Bone Marrow Cells:

Approximately 2-4 million of IL7R+ enriched cells were transduced in a 24-well with 70% of vector progeny received from the Takara “All Serotypes” kit, at a ratio of 1:9 with serum-less medium (Opti-MEM™). Spinoculation at 1200×g for 45 minutes was performed and 2 hours after initiating the transduction, full medium (MEM-alpha) containing 10% FBS was added at 3× volume excess. Cells were harvested 3 days post transduction.

### RNA Isolation and RT-PCR Analysis:

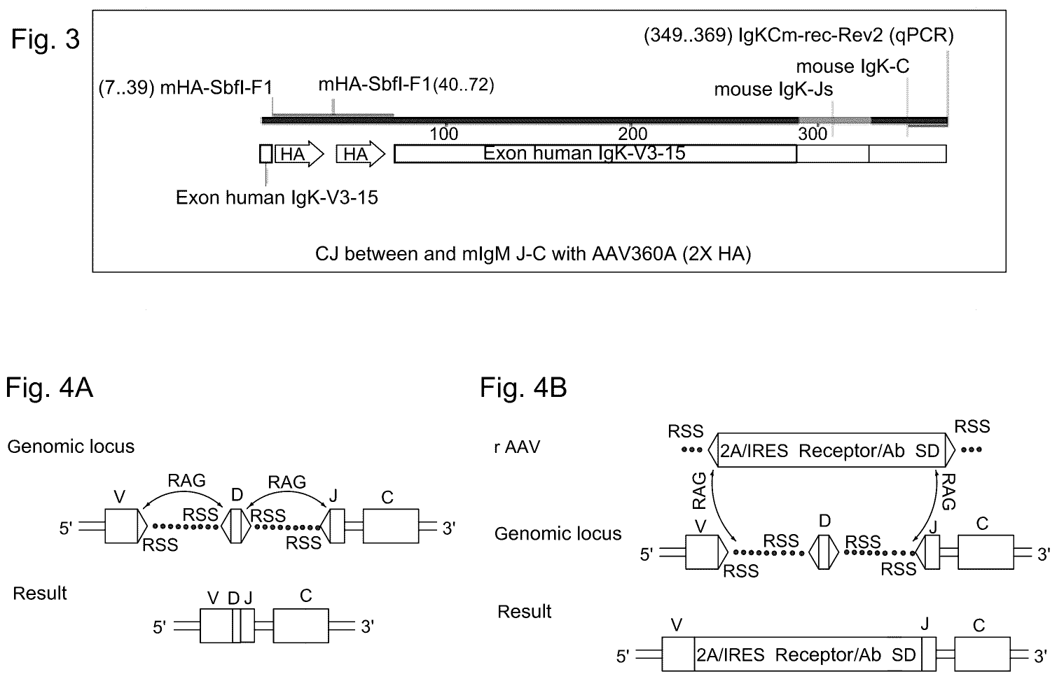

RNA isolation was performed using the Zymo Research Quick-RNA™ MicroPrep Kit according to the manufacturer's instructions. Reverse Transcription reactions were performed with 100-1000 ng total RNA, using 100 pmol oligo-dT as primer, 200 U of RNase H-minus M-MLV-point mutant (cat #M3682 by Promega), at 50° C. Typically, about 5% of the reverse transcription reaction was taken for PCR in 20 μl total volume, using standard conditions, a regular Taq polymerase ready-mix (cat #EZ-3006, by HyLabs) for 35 cycles of reaction, followed by 7 minutes tailing at 72° C. Primer locations and sequences are illustrated in FIG. 3.

mHA-SbfI-F1 primer as denoted by SEQ ID NO: 21
CTTACCCCTATGATGTGCCTGACTACGCTTGCA.
IgKCm-rec-Rev2 primer as denoted by SEQ ID NO: 22
GGATGGTGGAAGATGGATAC.

RT-PCR products were cloned into a TA cloning vector: pGEMT (cat #A3600, by Promega). Sequencing was then performed at the inter-departmental life sciences unit (IDRFU). Sequence alignment and analysis was performed by licensed SnapGene 4.1.5 software.

Intrathymic Injection of Cells Stained Fluorescently with CFSE:

About 1*10⁶ Jurkat-cells stained with CFSE (Carboxyfluorescein succinimidyl ester) were intrathymically injected with ultrasound guidance using 30-gauge insulin syringes (Tuckett A Z et al., J Hematol Oncol. 2017 16; 10 (1): 109; Tuckett A Z et al., Ultrasound Med Biol. 2015; 41:1105-1111) to 6-8 weeks old C57BL/6 mice. The thymus was isolated immediately after injection and the fluorescent signal from CFSE stained cells was detected using Maestro imaging system.

### Intrathymic Injection of Luciferase Transduced Cells:

Murine bone marrow cells were obtained from 6-8 weeks old C57BL/6 mice and used either fresh (BM) or after enrichment for Lin-cells (Lin−). Cells were transduced retrovirally to express Luciferase. 1E6 transduced cells were intrathymically injected using ultrasound guidance with 30 gauge syringes to 6-8 weeks old C57BL/6 mice. The signal of luciferase was detected after injection of Luciferin using the Biospace imaging system.

### Example 1

### V(D)J Targeting Strategy

VDJ targeting in accordance with the present invention, employs rAAV as well as the natural process of V(D)J recombination, taking place in developing lymphocytes. In natural V(D)J recombination, during T and B cell maturation, the genes to encode the two chains of a TCR or an Antibody are rearranged in a nearly random fashion from V and J gene segments (in a and γ TCR chains and Antibody light chains) or V, D and J segments (in β or δ TCR chains or Antibody heavy chain). V(D)J recombination is catalyzed by the RAG complex, composed of the RAG1 and RAG2 proteins as illustrated in FIG. 4A. RAG recognizes recombination signal sequences (RSSs) and recombines them to join together appropriate gene segments. RAG activity is associated with genomic instability and therefore, the inventors refrain from overexpressing RAG, but rather rely on its endogenous expression only. Flanking of a receptor/Antibody gene with appropriate RSSs, as demonstrated by FIG. 4B, allows its recognition as a substrate of RAG-mediated targeted insertion into the endogenous TCR/Antibody chain locus, but only if the vector transduces appropriate lymphocyte precursors, in which RAG is active. Recombination events involving the two flanking RSSs may be sequential rather than parallel, much as chromosomal D to J recombination naturally precedes V to DJ recombination. The identity of the endogenous chain being targeted depends on the lymphocyte precursor being transduced and on the RSS pair being used. The targeted cassette includes a sequence upstream to the receptor/Antibody gene which codes a 2A peptide or an IRES in order to avoid formation of protein fusions with preceding segments while still utilizing the strong endogenous promoter. A splice donor (SD) may further be provided downstream of the receptor/Antibody gene to facilitate utilization of the endogenous C segment(s) upon integration, transcription and splicing. More specifically, the SD is required to allow class switching of targeted Antibodies. In particular, an Antibody targeting cassette entails: RSS-2A/IRES-V_{L}J_{L}C_{L}-2A-V_{H}D_{H}J_{H}-SD-RSS, or alternatively encodes a single chain variable fragment or a bi-specific or tri-specific antibody.

A TCR targeting cassette entails: RSS-2A/IRES-VαJαCα-2A-VβDβJβ-SD-RSS, or alternatively encodes a TCRγδ.

A CAR targeting cassette entails RSS-2A/IRES-CAR-Stop-SD-RSS.

VDJ targeting relies on the ability of the RAG complex to act in trans, i.e. to recombine RSSs found on different molecules. Indeed, by its evolution, structure and mechanism of action, RAG is a DNA transposase. RAG was shown capable of inducing the transposition of DNA segments in trans both in vitro and in vivo. VDJ targeting further relies on rAAV transduction since rAAV is proficient in transducing many different tissues in vivo and several rAAV variants can efficiently transduce HSCs and lymphocytes [12].

### Example 2

### Vector Design

### RSS Choice

VDJ targeting in accordance with the invention can be used to integrate desired transgenes in any TCR chain (α, β, γ or δ) or Antibody chain (heavy, κ or λ) and between different segments in any chain. The identity of the endogenous chain being targeted depends on the lymphocyte precursor being transduced and on the RSSs flanking the transgene cassette. An RSS is composed of a conserved nonamer and a conserved heptamer separated by a variable spacer. The length of the spacer is either 12 bp or 23 bp (sometimes 22). The “12/23 rule” dictates that RAG will recombine an RSS that has a 12 bp spacer only with an RSS that has a 23 bp spacer and vice versa. Specific sequence features may further cause a particular “12 RSS” to recombine more efficiently with some “23 RSSs” but not with others, and this may underlie the “beyond 12/23 rule” preventing VJ rearrangements (skipping D) in TCR β and δ chains. Therefore, in some embodiments, the transgene cassettes is flanked either by “same” RSSs (two 12 RSSs or two 23 RSSs), or by an incompatible pair of a 12 RSS and a 23 RSS.

According to some embodiments, in the specific vector configuration for VDJ targeting in the B lineage, a cassette flanked by two “outward facing” 12 RSSs is targeted to integrate between a V and a J segment of the heavy chain (FIG. 5A). In some further embodiments, the specific vector configuration for VDJ targeting in the T lineage, a cassette flanked by two “downstream facing” 23 RSSs is targeted to integrate between a D and a J segment of a β chain (FIG. 5B). The upstream 23 RSS on the vector is of the “V type” and the downstream 23 RSS on the vector is of the “D type”, in order to preclude integration between two genomic J segments, according to the beyond 12/23 rule. Subsequent natural V to D rearrangement brings the V promoter to the proximity of the enhancer and allows robust cassette expression. It is possible to avoid the requirement for additional, naturally occurring V(D)J rearrangements by coding an enhancer or a promoter (but not both) as part of the cassette.

Importantly, RSS configuration is essential but not sufficient for desired targeting. VDJ targeting takes place only if a vector with the appropriate RSS configuration transduces the appropriate lymphocyte at the appropriate developmental stage, because genomic RSS accessibility is determined by developmentally regulated transcription and epigenetic factor. The first constructs described herein target the heavy chain locus in B cell and the β chain locus in T cells. When targeting in vitro differentiating lymphocytes, the targeting occurs early during development (Pro-B cells and DN1/2 cells respectively). Targeting between V and J segments or between D and J segments must be induced before the natural D to J rearrangement which precedes the V to DJ rearrangement in these cells. Targeted cells does not express a pre-BCR or pre-TCR, yet TCR/BCR transgenic mouse data imply that they nevertheless successfully pass all developmental checkpoints. In subsequent experiments, the rates and consequences of targeting the light chains in B cells and the α chain (and later γ or δ chains) in T cells are assessed. This requires targeting later during in vitro differentiation (when targeting in vitro) and using appropriately oriented 12 and 23 RSSs.

### Inclusion of Constant Regions or Splice Donors

In a specific and non-limiting configuration for VDJ targeting in the B cell lineage, a heavy chain constant region is not encoded but instead a downstream splice donor (SD) is included to allow the utilization of endogenous constant region. The cassette encodes an Antibody light chain (V_{L}J_{L}C_{L}) and a variable region of a heavy chain (V_{H}D_{H}J_{H}) separated by a 2A peptide (V_{L}J_{L}C-2A-V_{H}D_{H}J_{H}-SD). Upon integration, transcription and splicing, the transgene is first expressed as a BCR on naïve B cells. Following antigen-induced activation and affinity maturation the transgene is also expressed as antibodies of different classes being secreted from plasma cells.

In the T cell lineage, two different specific configurations for VDJ targeting are used, specifically, TCR and CAR.

More specifically, when targeting a transgenic TCR cassette to the TCR β chain locus, a heavy chain constant region is not encoded but instead it includes a downstream splice donor (SD) to allow the utilization of endogenous constant region. The cassette encodes a TCR α chain (VαJαCα) and a variable region of a β chain (V_{β}D_{β}J_{β}) separated by a 2A peptide (V_{α}J_{α}C_{α}-2A-V_{β}D_{β}Jβ-SD). rAAV coding capacity is limited, and therefore omitting CB in favor of a SD is beneficial. In addition, without being bound by theory, it seems that the SD inclusion allows a more natural-like expression with increased RNA stability and subsequent translation.

When targeting a CAR cassette to the TCR β chain locus, the full CAR gene is encoded, ending with a stop codon, and only then a SD (additional variants include a synthetic polyadenylation site).

Inclusion of Minimal Promoters and/or Splice Acceptors

In a specific and non-limiting configuration for VDJ targeting in the B cell lineage, a splice acceptor (SA) or a minimal promoter (MP) may be included. The cassette encodes an Antibody light chain (V_{L}J_{L}C_{L}) and a variable region of a heavy chain (V_{H}D_{H}J_{H}) separated by a 2A peptide (SA-V_{L}J_{L}C-2A-V_{H}D_{H}J_{H}-SD). Upon integration, transcription and splicing, the transgene is first expressed as a BCR on naïve B cells. Following antigen-induced activation and affinity maturation the transgene is also expressed as antibodies of different classes being secreted from plasma cells. Similar example with a minimal promoter may be: MP-V_{L}J_{L}C-2A-V_{H}D_{H}J_{H}-SD.

In the T cell lineage, two different specific configurations for VDJ targeting are used, specifically, TCR and CAR.

More specifically, when targeting a transgenic TCR cassette to the TCR β chain locus the cassette may further include an upstream splice acceptor (SA). The cassette encodes a TCR α chain (V_{α}J_{α}C_{α}) and a variable region of a β chain (V_{β}D_{β}J_{β}) separated by a 2A peptide (SA-V_{α}J_{α}C_{α}-2A-V_{β}D_{β}J_{β}-SD). Similar embodiments using a minimal promoter may include (MP-V_{α}J_{α}C_{α}-2A-V_{β}D_{β}J_{β}-SD). In some embodiments, the splice acceptor (SA) may be SA 97.66 having the nucleic acid sequence as denoted by SEQ ID NO: 26. In some embodiments, the splice acceptor (SA) may be SA EF1a having the nucleic acid sequence as denoted by SEQ ID NO: 31. In some embodiments, the splice donor (SD) may be SD mIGHJ1/J4 having the nucleic acid sequence as denoted by SEQ ID NO: 32. In some embodiments, the splice donor (SD) may be SD IgHJ3 SD 98.84 having the nucleic acid sequence as denoted by SEQ ID NO: 33.

In some embodiment, the minimal promoter may be a Variant of IGH minimal promoter having the nucleic acid sequence as denoted by SEQ ID NO: 34. In some embodiment, the minimal promoter may be an IgK Minimal Promoter having the nucleic acid sequence as denoted by SEQ ID NO: 35.

### IRES/2A Coding Sequences

In some embodiments, the targeted cassettes include a sequence upstream to the receptor/Antibody gene which codes a 2A peptide or an IRES in order to avoid formation of protein fusions with preceding segments while still utilizing the strong endogenous promoter. In some embodiments, the IRES represents the first choice because subsequent expression is independent of junctional diversity introduced by terminal deoxynucleotidyl transferase (TDT) during RAG induced rearrangements. The IRES is preceded by a 3-frame stop to prevent ongoing translation. 2A peptides only induce ribosomal skipping if the reading frame is preserved (roughly ⅓ of events). Nevertheless, when active, 2A peptide can allow very high expression levels of an integrated transgene. Therefore, some of the vectors encode a 2A peptide to assess which design allows maximal expression. In addition, many of the vectors encode several peptides (e.g. two Antibody chains) and these are separated by 2A peptides. A non-limiting example of a vector design with a preceding IRES and an intervening 2A peptide may be: (RSS-IRES-V_{L}J_{L}C-2A-V_{H}D_{H}J_{H}-SD-RSS).

### Inclusion of an Inducible Suicide Gene

Many immunotherapy targets are self-antigens, and therefore sustained autoimmunity from lymphocytes directed against these autoantigens is a serious problem for all lymphocyte engineering approaches, including for VDJ targeting described herein. The optimal vector configuration for facilitating VDJ targeting is evaluated by incorporation of an inducible suicide gene for the specific elimination of targeted lymphocytes (when expression is found to be associated with adverse outcomes). In particular, an upstream caspase gene fused to an inducible dimerization domain (iCasp9) is added to the cassette. The default is the expression of an inactive monomeric caspase. However, if an adverse outcome is seen and quick shutoff is desired, the addition of a small membrane permeable molecule allows the dimerization of the caspase and the subsequent induction of apoptosis. Importantly, similar systems are successfully used for the elimination of genetically modified T cells in multiple ongoing clinical trials (e.g. NCT01875237). Apoptosis induced by caspase dimerization is independent on cell cycling, it is quick, non-immunogenic and has little bystander effect compared to common alternatives for induced cell death. An example of a vector design with an inducible suicide gene is: (RSS-IRES-iCasp9-2A-V_{L}J_{L}C-2A-V_{H}D_{H}J_{H}-SD-RSS).

Inclusion of miRNAs

The optimal vector configuration for facilitating VDJ targeting is evaluated followed by testing designs incorporating one of several alternative and non-mutually exclusive miRNAs:
a) miRNA against non-targeted chains, e.g. in order to target a full TCR gene (TCRα-2A-TCRβ) into the TCRβ chain locus, miRNA against the TCRα chain is used to prevent cross-dimerization.
b) miRNAs against the non-targeted allele; knockdown of the other allele is desired only if natural allelic exclusion is absent or incomplete, for example when introducing a CAR gene rather than a TCR and thus not providing all natural signals for allelic exclusion.
c) miRNA against the immune checkpoint protein PD1 in T cells; this prevents tumor tolerance but may also increase the risk of adverse autoimmunity and therefore it is used in conjunction to an inducible suicide gene as described above.
d) miRNA against an apoptotic gene; this may prevent central tolerance by negative selection if targeting an autoantigen but it also increases risk of sustained adverse autoimmunity and therefore it is used in conjunction to an inducible suicide gene as described above.

Importantly, the miRNA does not require a vector borne promoter but instead can be efficiently processed from an intron upon transgene integration and transcription. An example of a vector design with a miRNA and an inducible suicide gene is: (RSS-IRES-iCasp9-2A-V_{L}J_{L}C-2A-V_{H}D_{H}J_{H}-SD-miRNA-RSS).

rAAV Capsid and Genome

Several rAAV serotypes can efficiently transduce HSCs and lymphocytes. The rAAV6 serotype is used for most in vitro VDJ targeting assays, because it is the most widely used serotype in lymphocytes to date [12]. However, additional serotypes are also tested including HSC derived AAV and rAAV8 that was found efficient, and superior to lentivectors, at in vivo intrathymic transfection in both mice and macaques [16]. The transgene cassettes is encoded in self-complementary rAAV (scAAV), which allows the RSSs to quickly obtain the dsDNA configuration that is recognized by RAG. ScAAV coding capacity is just over 2 Kb. Therefore, for targeting of longer genes or several genes (e.g. when introducing an inducible suicide gene), the cassettes are encoded in single stranded AAV (ssAAV). ssAAV is processed in the target cell into dsDNA forms but with delay and efficiency loss compared to scAAV.

### Example 3

### Developing VDJ Targeting of Ex Vivo Differentiating Lymphocytes

### Assays in Readily Transfected Cell Lines

VDJ targeting is first demonstrated in episomal and integrated reporter systems in readily transfected cell lines, as illustrated by FIG. 6. Activity of the transgenic RAG in the cell lines is verified using a reporter system where RAG induced recombination in cis leads to GFP expression (FIG. 6A). VDJ targeting is then demonstrated using a donor vector coding for an RSS-flanked promoterless GFP and an either episomal or integrated reporter coding for RFP flanked by RSSs which are compatible with the donor RSSs (FIG. 6B). VDJ targeting rates is normalized to transfection efficiency (measured using a canonical GFP expression vector) and to RAG activity and compared to a negative control where the RAG expressing plasmids are not co-transfected. Analogous reporter system is used in human cell lines (typically HEK293) and mouse cell lines (typically NIH3T3). The appropriate human or mouse RAG proteins is expressed in each system. RSS sequences are chosen that are highly used in natural V(D)J recombination in developing T cells or B cells in the respective organism. Different donor and reporter plasmids encode different RSS sequences at different relative orientations to assess compatibility and maximize efficiency. Several RSS choice are tested for their ability to help in targeting integration to one chain but not another chain (e.g. to the β and not δ chain), and also to one pair of segments and not another pair (e.g. target integration between D and J and not between two different J segments). Episomal assays (co-transfection of donor and reporter) is used for quick initial readout, but ultimately, selected cell clones harboring a single integrated reporter provide a clearer result (targeted cells are not only green, they are also not red).

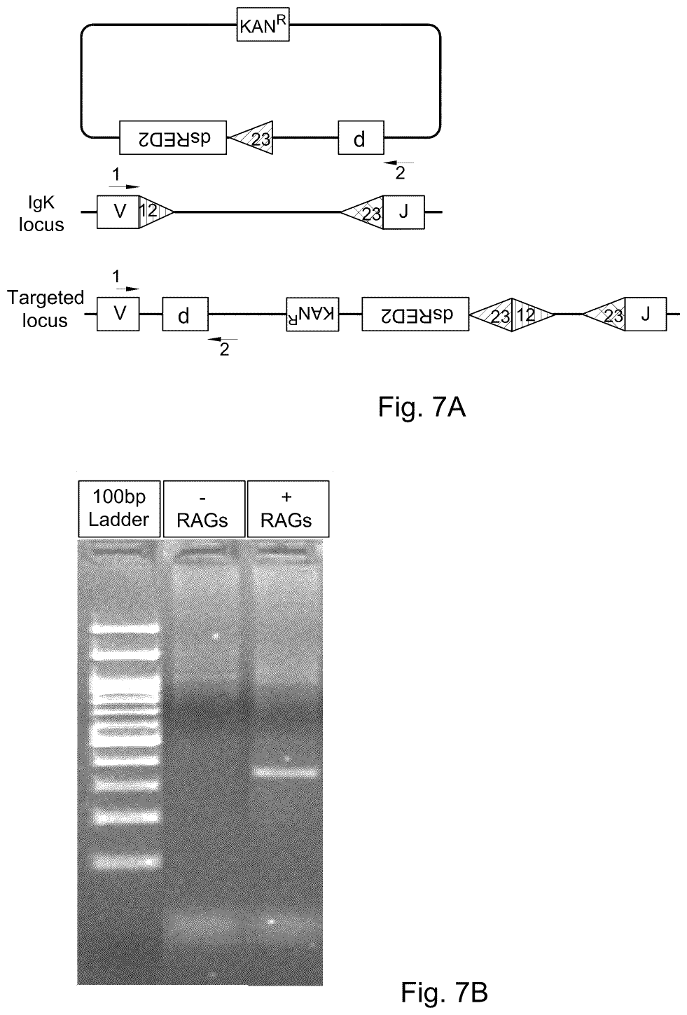

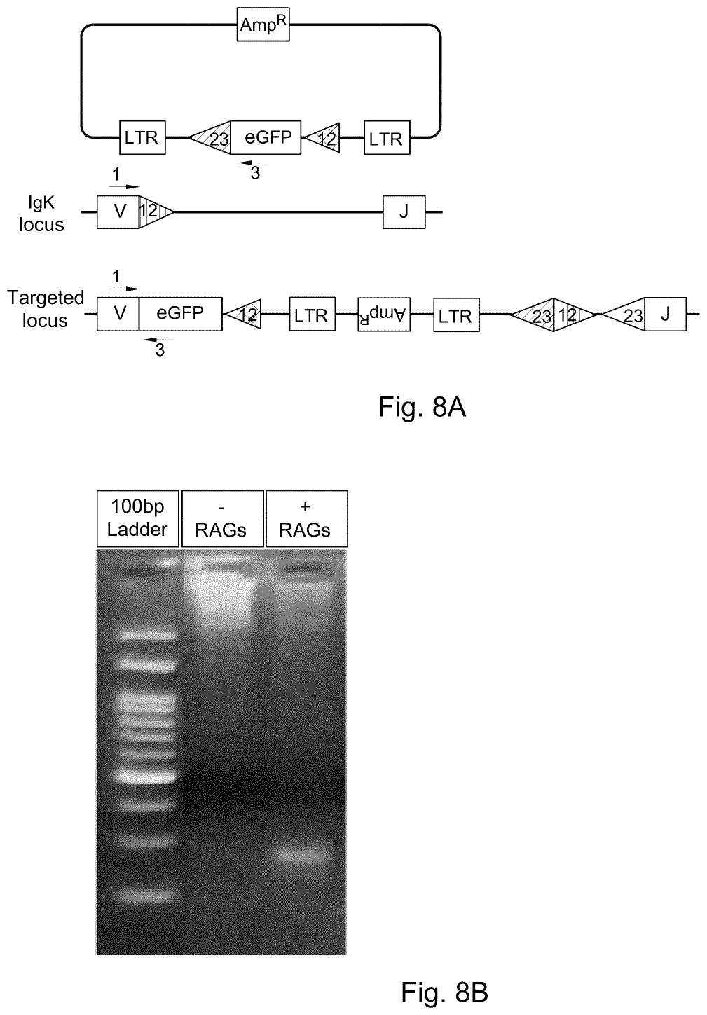

Examples of integrated reporter systems transfected in HEK293 cell lines are provided in FIG. 7 and FIG. 8. VDJ targeting of a single-RSS construct containing dsRED reporter gene, was performed toward the IgK locus of HEK293T cells (FIG. 7A). PCR analysis demonstrated that upon co-transfection with plasmid encoding RAG1 and RAG2, the dsRED2 cassette was successfully incorporated between the V and J segments of the IgK locus (FIG. 7B).

VDJ targeting of a dual-RSS construct containing GFPi-redless reporter gene, was performed toward to the IgK locus in HEK293T cells (FIG. 8A). PCR analysis demonstrated that upon addition of plasmids encoding RAG1 and RAG2, the GFPi-redless cassette was successfully incorporated between the V and J segments of the IgK locus of T cells (FIG. 8B).

### Assays in Transformed Lymphocyte Cell Lines:

Following, selected reporter vectors are then used to assess VDJ targeting rates in lymphocyte cell lines which express RAG either constitutively or inducibly. In particular, VDJ targeting is assessed in the Sup-T1 T-ALL cell line, which expresses RAG at a high basal level, and in V-Abl transformed pro-B cell lines (Lescale C et al., Nat Commun. 7:10529 (2016)) and Pre-B cells lines (Yin B, et al. J Exp Med. 206 (12): 2625-2639 (2009)), which express RAG upon induction by the V-Abl kinase inhibitor. In these cells, the RSS compatibility is examined in the lymphocyte setting, and in addition, the effect of different delivery methods on the rates of VDJ targeting is compared. In particular, VDJ targeting rates after co-delivery of the same donor and reporter pair using either plasmid electroporation (Neon™), lentiviral vectors or rAAV vectors are compared, where each rate is normalized to transduction rates of a GFP positive control using the respective delivery method. For rAAV vectors, VDJ targeting propensity of self-complementary AAV (scAAV) and single stranded AAV (ssAAV) and of serotypes rAAV6 and rAAV8 are compared as well as HSC derived rAAV.

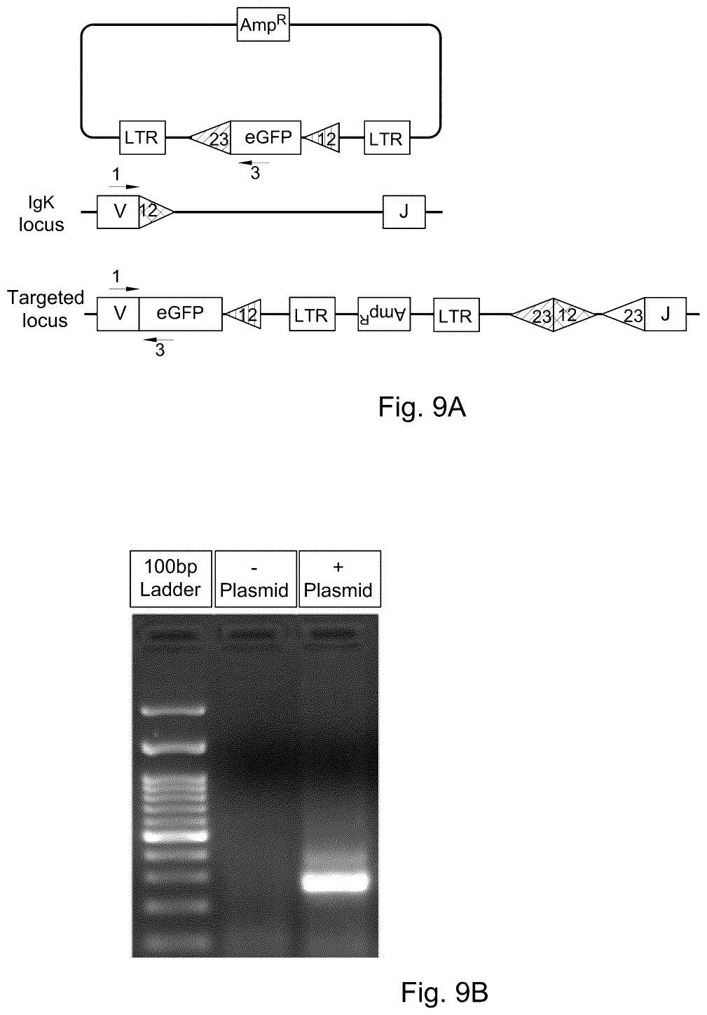

An example of VDJ targeting into inducibly differentiating B cells is provided in FIG. 9. V-Abl immortalized murine Pro B cells were transfected with the GFPi-redless plasmid containing a 23-consensus RSS and a 12-consensus RSS. Cells further received Imatinib for induction of differentiation as schematically represented in FIG. 9A. PCR analysis demonstrated that upon addition of the plasmid encoding GFPi-redless, the GFPi-redless cassette was successfully incorporated between the V and J segments of the IgK locus of differentiating B cells (FIG. 9B).

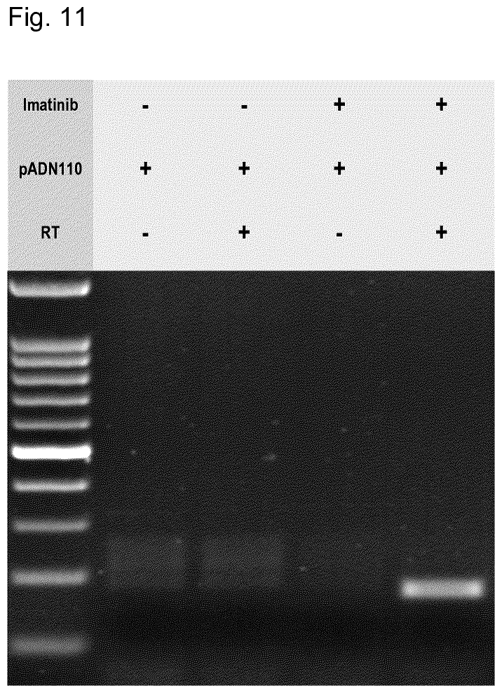

In addition, a gene coding for an antibody was integrated by VDJ targeting into the IgK locus of inducibly differentiating B cell progenitors. The inventors used a cell line of immortalized pro-B cells that can be induced to differentiate and express RAG upon administration of the v-Abl kinase inhibitor Imatinib. Electroporation was performed using a plasmid coding for the anti-RSV antibody Palivizumab flanked by appropriate RSSs (see illustration in FIG. 10). Analysis by Reverse transcription reaction (see more details in the Experimental procedure section) shows that integration into the IgK locus and subsequent expression occurred only upon addition of Imatinib (see FIG. 11).

### Reporter Assays in In Vitro Differentiating Primary Cells

Next, the rates of VDJ targeting is assessed in primary developing lymphocytes in vitro. Initially, GFP coding donor vectors are used, as GFP allows an easily quantifiable readout and is highly suitable for protocol optimization. However, follow-up experiments, targeting receptor genes (TCR/CAR/BCR), may reveal interesting discrepancies with the GFP experiments because receptor presentation is known to affect subsequent lymphocyte differentiation. Therefore, some of the vectors target both a GFP marker and a receptor (detailed below), separated by a 2A peptide. VDJ targeting rates are assessed in lymphocytes differentiated from three diverse pluripotent cell sources: mouse Embryonic Stem Cells (Kucerova-Levisohn M, et al., J Vis Exp. (92): e52119. (2014)), mouse Hematopoietic Stem cell (HSCs), and human Umbilical Cord Blood Hematopoietic Stem cells (UCB HSCs) [7]. The vectors for transfection of mouse and human cells codes the respective RSS sequences and have appropriate codon usage optimization.

In the T cell lineage, RAG is naturally expressed during the maturation of double negative 1 (DN1 through ISP) thymocytes, and in the B cell lineage, it is expressed during differentiation from Pro-B cell to immature B cell. In vitro, pluripotent cells are differentiated to T-cell on a support of OP9-DL1 cells providing Notch differentiation signals, and pluripotent cells are differentiated to B cells on a mesenchymal stem cell-conditioned medium containing SCF, G-CSF, FLT3 ligand, and IL-7. Precursors of different stages are transfected with an rAAV6 or rAAV8 vector coding for a GFP gene flanked by RSSs. Subsequently, additional differentiation is facilitated to allow for RAG expression and VDJ targeting. Real time PCR and flow cytometry are both used to assess the targeting rate. For each type of differentiating culture, the composition and differentiation status of the culture are assessed daily by flow cytometry. rAAV transfections are performed at different days opting to find the timing that allows the highest rates of VDJ targeting. The first transfection time point in the T lineage is when the proportion of double negative 1 (DN1) cells is peaking, per flow cytometry (allowing for DJ recombination at the β TCR locus). The last transfection time point in the T lineage is when the proportion of ISP cells is peaking (allowing for VJ recombination at the α TCR locus). The first transfection time point in the B lineage is when the proportion of pro-B cells is peaking, per flow cytometry (allowing for DJ recombination at the heavy chain locus). The last transfection time point in the B lineage is when the proportion of immature B cells is peaking (allowing for VJ recombination at a light chain locus). For both lineages, the optimal transfection time point is evaluated followed by examination of the efficacy of different MOIs (1E9 vg/mL through 1E11 vg/mL) and construction of a dose response curve.

Targeting In Vitro Differentiating B Cells from IL7R+ Enriched Primary Bone Marrow Using AAV

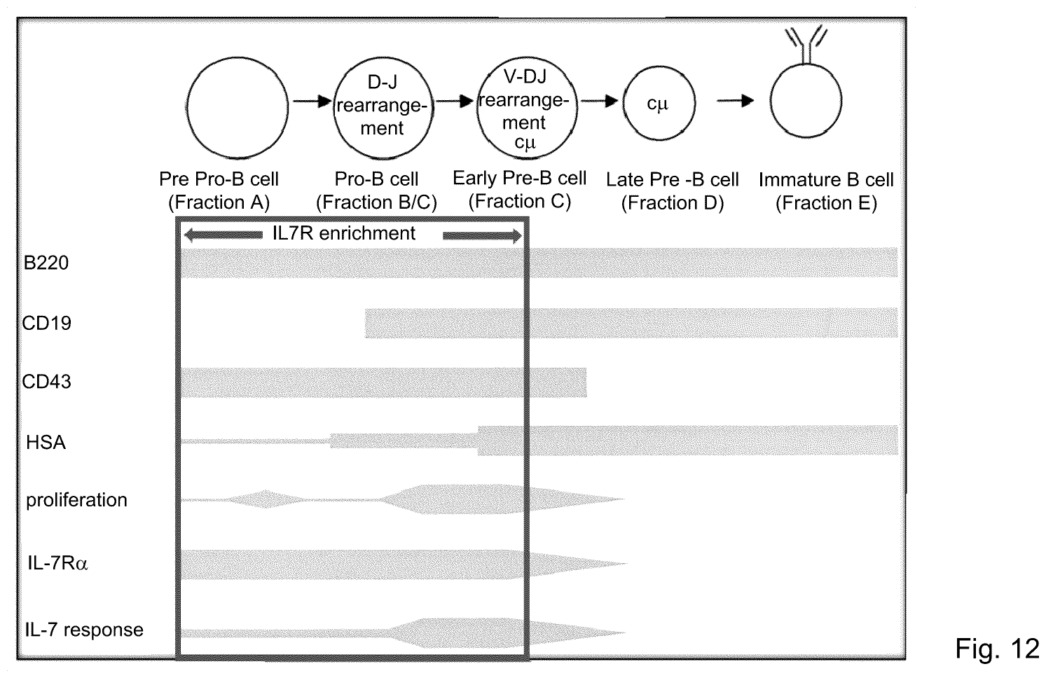

IL7R positive cells in the bone marrow correspond to B-cell progenitors on the verge of V(D)J recombination as described in FIG. 12. Bone marrow were thus extracted from mice. Enrichment for IL7R positive cells was then performed, in vitro differentiation was facilitated and the cells were transduced with an AAV vector coding for a VDJ-targeting construct, the dsAAV 360-A viral vector (the bacterial plasmid used in the preparation of the AAV vector, see more details in the Experimental procedure section). The resulting products were analyzed by RT-PCR, cloned into bacterial plasmids and subjected to Sanger sequencing as detailed in the Experimental procedure section.

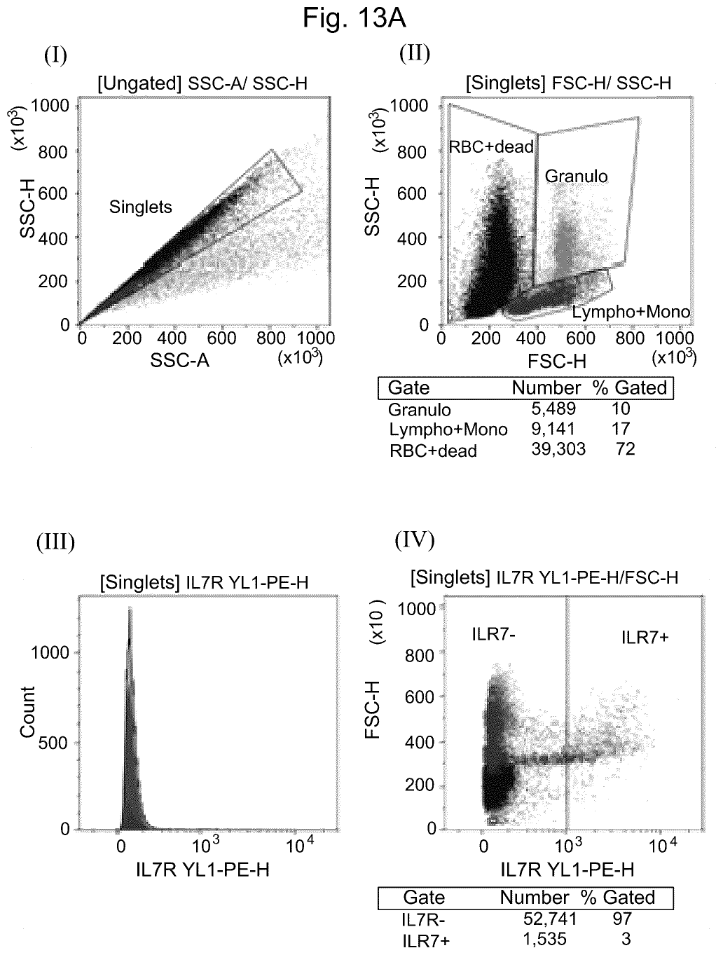

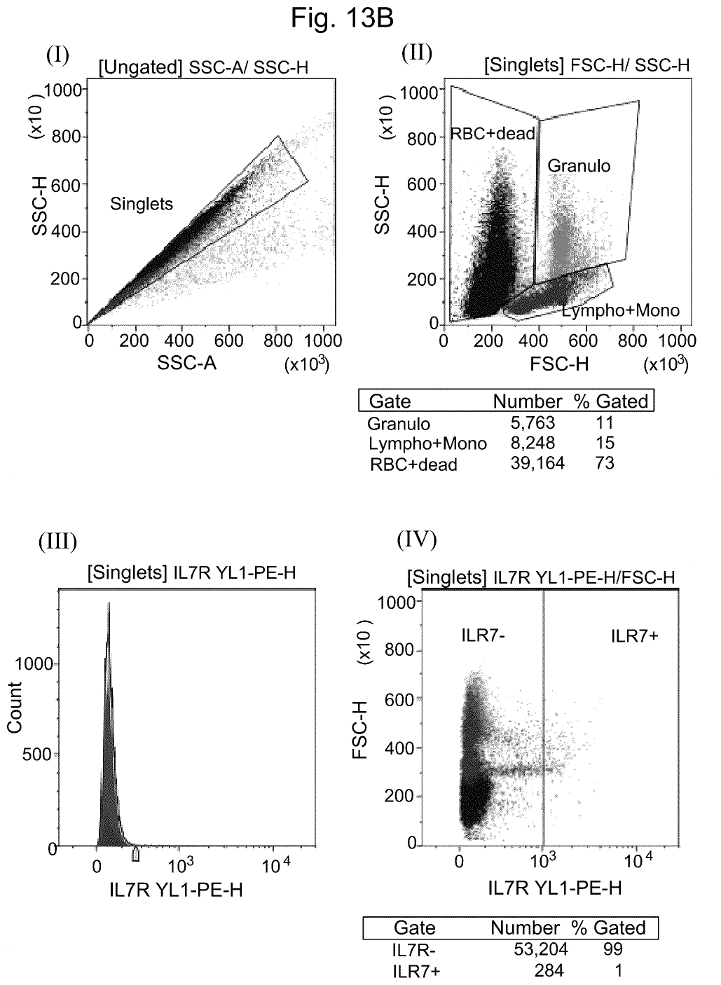

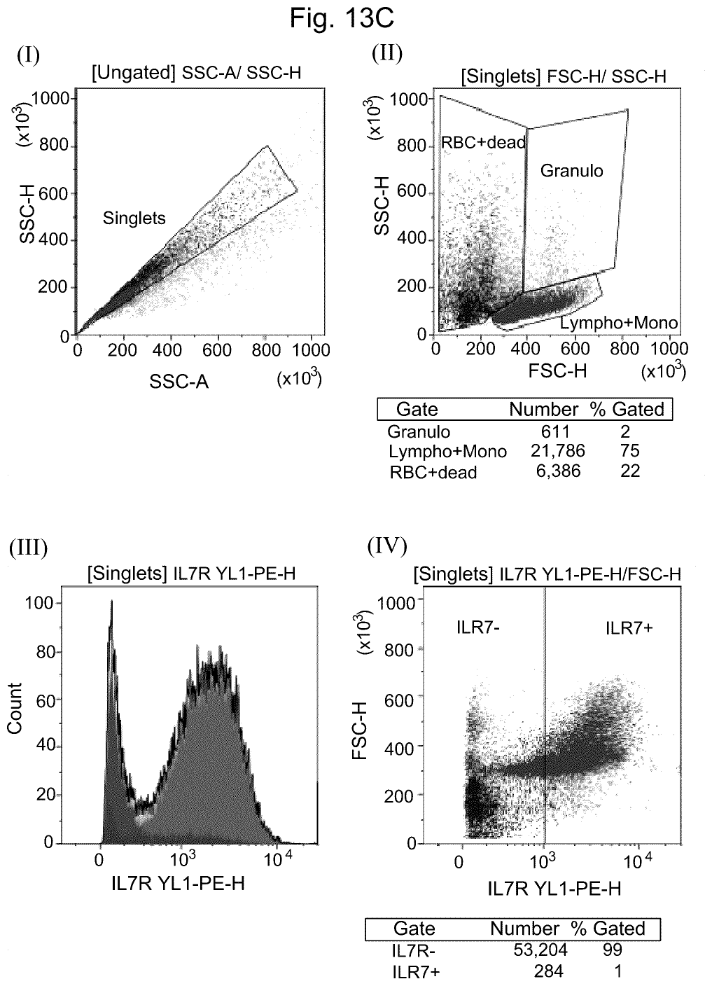

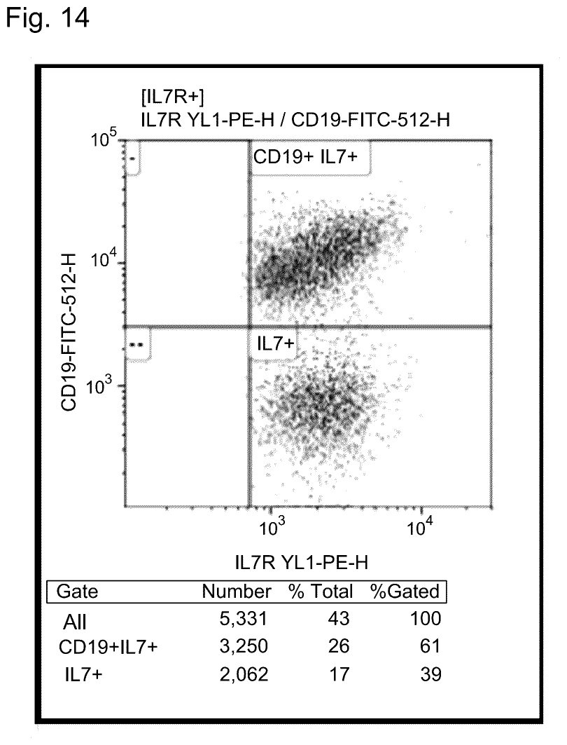

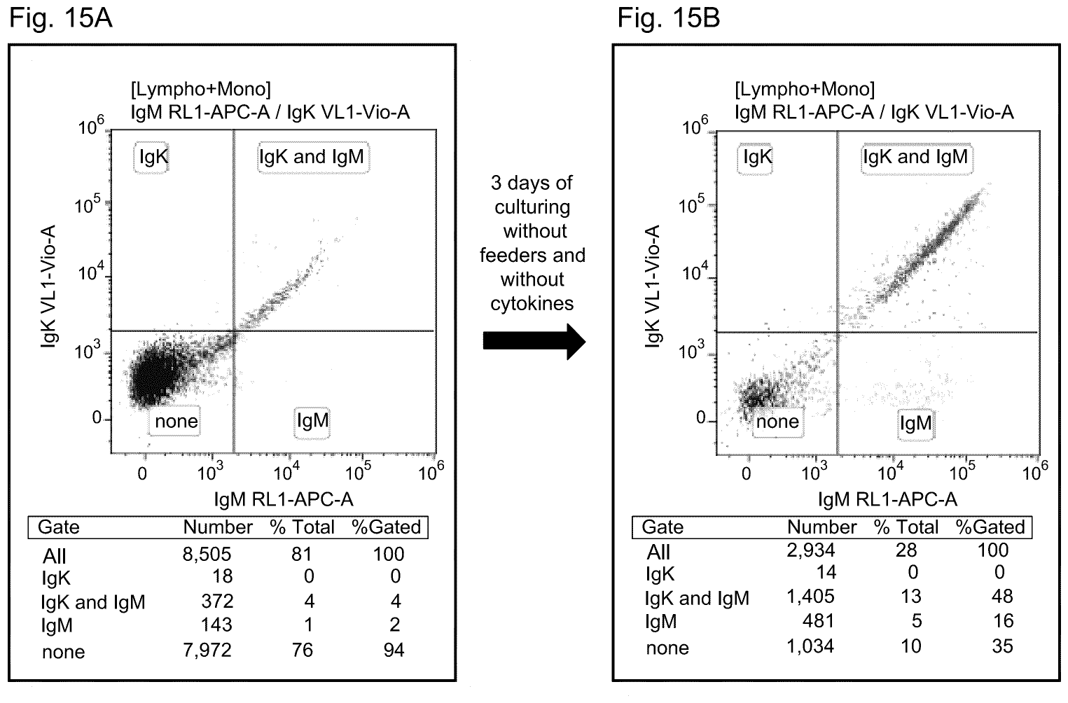

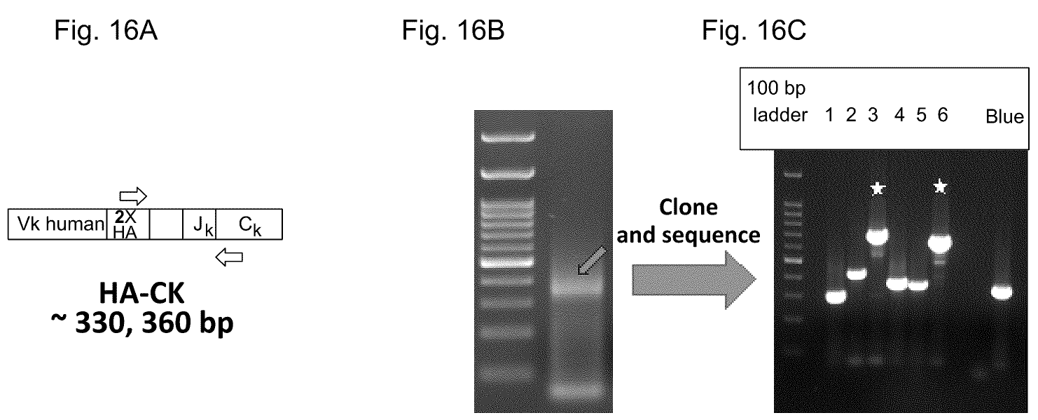

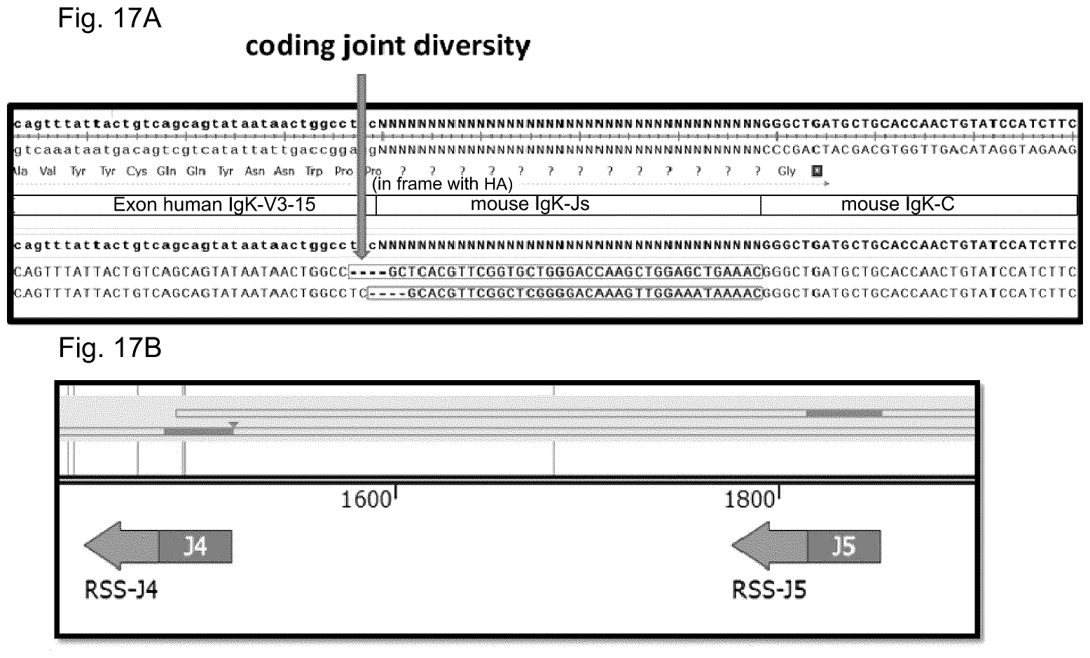

More specifically, for VDJ targeting of B cell progenitors, IL7R cells were enriched. It appears that IL7R+ enriched bone marrow cells exhibited a marked increase in purity. Typical IL7R enrichment was about of 20-30 fold (see FIG. 13A-13C (i-iv)). As shown in FIG. 14, following isolation, ˜60% of the cells expressing the IL7 receptor also express the CD19 B-cell marker. Within 3 days of culture, IL7 receptor-positive enriched lymphocytes exhibit a dramatic increase of differentiation rate, as exhibited by expression of both IgM and IgK while IgM precedes IgK (FIG. 15). IL7R-enriched bone marrow cells were than transduced with the dsAAV 360-A viral vector as described in experimental procedures. Trans-recombination products were detected in transduced cells by Reverse Transcription followed by two rounds of PCR (see FIG. 16A, 16B, 16C). Sanger sequencing of two separate clones was performed and showed junctional diversity at the coding joint, a unique attribute of RAG mediated integration. The two coding joints map to IgK-J4 and IgK-J5 are illustrated in FIG. 17A-17B. These results illustrate successful V(D)J targeting of an immunoglobulin light chain segment in primary B cells.

### Receptor/Antibody Targeting in In Vitro Differentiating Primary Cells

The optimal parameters of VDJ targeting are evaluated using the GFP construct in in vitro differentiating T cells and the protocol is subsequently applied to the integration of several different receptors of clinical relevance.

The following receptors are targeted:
a) an anti CD19 CAR coding construct, shown to be highly effective in treating B cell malignancies;
b) an anti-Tn-MUC1 CAR coding construct which targets a highly cancer specific glycoform; and
c) an NY-ESO-1-specific TCR construct. NY-ESO-1 is a cancer-testis antigen and early clinical data with the TCR concordantly shows good safety and efficacy profiles (Rapoport A P, et al. Nat Med. 21 (8): 914-921 (2015)).

Targeted receptors were chosen to maximize the therapeutic impact while minimizing the risk of sustained auto-immunity. Nevertheless, the risk remains and is subsequently mitigated by the introduction of inducible suicide genes. Targeting rates are validated using RT-PCR and flow cytometry. Flow cytometry is further used to determine the subclass distribution among the targeted T cells upon maturation. This is followed by in vitro functional assays, including: chromium release from labeled CD19/Tn-MUC1/NY-ESO-1-expressing cells as well as a cytokine release assay (Kunkele A, et al. Cancer Immunol Res. 3 (4): 368-379 (2015)). In addition, where available, the inventors demonstrate that stimulated T cells that were targeted while differentiating from HSCs obtained from cancer patients can lyse autologous Tumor cells.

Similarly, the optimal parameters of VDJ targeting are evaluated using the GFP construct in in vitro differentiating B cells and the protocol is subsequently applied to the integration of several different Antibodies of clinical relevance. The following antibodies are targeted:
(a) 3BNC117 an anti-HIV Antibody showing remarkable clinical efficacy (Scheid J F, et al. Nature. 535 (7613): 556-560 (2016)); (b) Adalimumab (Humira™) an anti-TNFα Antibody that is effective in many auto-immune diseases but is associated with a high cost and high incidence of anti-drug antibody formation (Bartelds G M, et al., JAMA. 305 (14): 1460-1468 (2011)); and
(c) PankoMab-GEX an anti MUC1 glycoform Antibody that is highly cancer specific and has a good safety profile according to recent clinical trials (Fiedler W, et al. Eur J Cancer. 63:55-63 (2016)). Targeting rates are validated using RT-PCR and flow cytometry. Flow cytometry is further used to determine the subclass distribution among the targeted B cells upon maturation. This is followed by in vitro B cell activation assay.

In both lineages, LAM PCR is used followed by next generation sequencing to assess off-target integration profile, and karyotyping is used to detect chromosomal aberrations.

Adoptive Therapy with Targeted Cells in Animal Models:

Well established mouse models are used to assess the in vivo potency and safety of selected in vitro targeted and differentiated cells. Efficacy of Anti-CD19 CAR targeted T cells is tested in the Raji Burkitt lymphoma bearing SCID-beige model, following CD80 and IL15 co-stimulation (Brentjens R J, et al. Nat Med. 9 (3): 279-286 (2003)). In particular, selective expansion of targeted cells leading to subsequent drop in CD19 counts and eradication of Raji tumors are evaluated in these mice. Efficacy of B cells targeted with anti-TNFα Antibody is also evaluated in a human TNFα over-expressing transgenic mouse. These TgTC mice develop a progressively erosive polyarthritis developed with many characteristics of human rheumatoid arthritis, including polyarticular swelling, impairment of movement, cartilage and bone erosion and synovial hyperplasia. Following adoptive transfer of targeted B cells, repertoire sequencing is used in order to monitor selective expansion and affinity maturation (somatic hypermutations as well as class switching) and to prevent arthritis in this mouse model.

Safety assessment is paramount in the in vivo experiments. For all in vivo studies, mice weight, appearance and behavior as well as assess plasma levels of AST enzymes, as markers of tissue toxicity are monitored and scored. A multiplex bead-based cytokine assay is also applied because cytokine storm is a common adverse effect of cancer immunotherapy. LAM PCR is performed for off-target integration analysis using plasma derived T-cells and post-mortem thymocytes. Finally, some mice are transplanted with targeted lymphocytes early in life and kept alive for 18 subsequent months to allow long term carcinogenicity assessment.

### Example 4

### Developing VDJ Targeting by In Vivo Vector Administration

Ex vivo engineering of lymphocytes and pluripotent cells is cumbersome, time consuming and expensive. VDJ targeting following in vivo rAAV injection allows scaling up and applying the treatment to a much wider population. Importantly, rAAV vectors have a better safety profile than lentiviral and retroviral vectors, commonly used for T cell engineering to date. rAAV vectors are also superior to lentivectors in the transduction of thymocytes in vivo. The rAAV constructs for in vivo assessment of VDJ targeting is similar to those used for in vitro assessment, using rAAV6 and HSC derived rAAV, as well as the rAAV8 serotype that was previously shown to be efficient in intrathymic transfection.

As described herein before, for safety validation, mice weight, appearance and behavior as well as assessment of plasma levels of AST enzymes, as markers of tissue toxicity are monitored and scored. LAM PCR is performed for off-target integration analysis using plasma derived T-cells and post-mortem thymocytes, and some mice are injected early in life and kept alive for 18 subsequent months to allow long term carcinogenicity assessment.

Intrathymic rAAV Injections

Intrathymic injections are most clinically relevant at an early age, prior to thymic involution. However, in humans, thymopoiesis may continue at least until age 40. T cells develop in thymus, thus, thymic injections may allow transduction of T cells progenitors undergoing VDJ recombination. Alternatively, in vitro differentiated T cell precursors may be transduced. The cells may then be infused either into the thymus or systemically and thymic migration and retention is then monitored.

### Transfection Positive Control

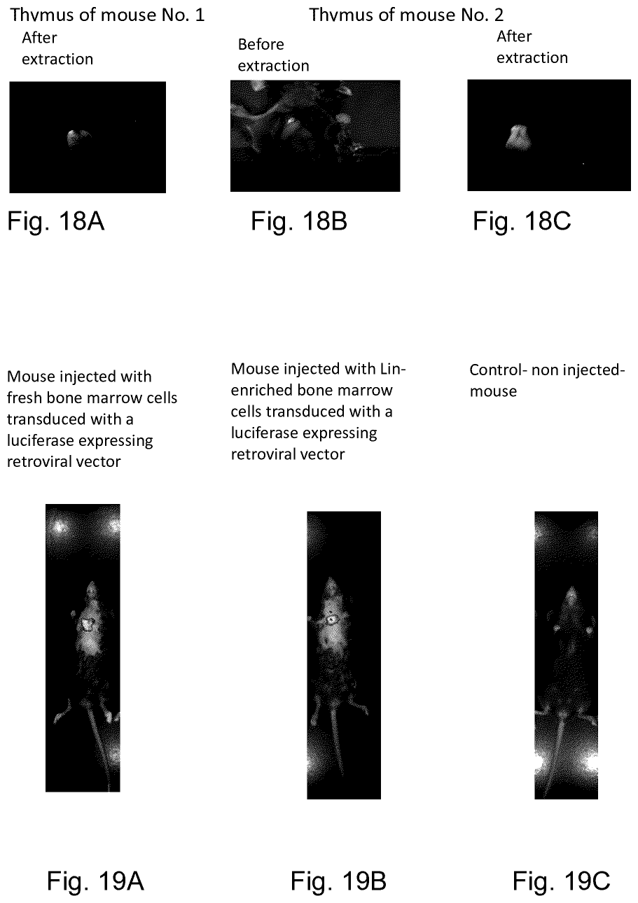

First, injections were performed to demonstrate the feasibility of cell injection into the thymus of a live animal. Then, luciferase-transduced cells were used to show that cell retention may be monitored by following a luciferase signal (see details in the Experimental procedure section). It appears that Jurkat cells stained with CFSE and injected intrathymically using ultrasound guidance in live mice were readily detected in mouse thymuses after post-mortem surgery (see FIG. 18A-18C).

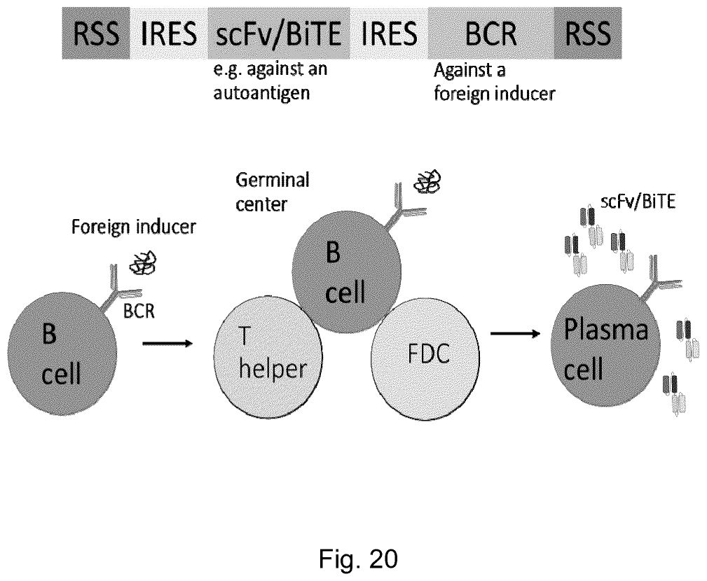

Next, fresh murine bone marrow cells, with or without enrichment for Lin-cells, were transduced with a retroviral vector encoding for luciferase and injected intrathymically using ultrasound guidance in live mice. Luciferase signal was readily detected in the thymuses of live mice following luciferin administration (see FIG. 19A-19C).

In a next step, doses of 1E11 to 1E12 rAAV vectors are injected directly into the thymus of 4-8 weeks-old mice. Canonical, promoter-driven, GFP-coding constructs are injected as a positive control, and transfection rates are estimated using RT-PCR and flow cytometry. Based on Moreau et al. [16], by day 3 post-transfection, rates of GFP+ cells are assessed among all thymocyte populations as well as epithelial cells and dendritic cells. The percentages of transduced thymocytes within the DN (CD4−CD8−), DP (CD4+CD8+), and SP mature (CD4+CD8− and CD4−CD8+) subsets reflect their overall thymic distribution, as expected. Monitoring rates of GFP+DP cells at day 10 post-transfection, an increase subsequent to differentiation of immature transduced progenitors is demonstrated as expected, however, DN transduced thymocytes are still detected at significant rates at days 10 and 30 post-injection. The migration of differentiated targeted cells is further examined into the periphery. In particular, flow cytometry and RT-PCR are used to estimate rates of targeted cells in the spleen and in the lymph nodes at days 3, 10 and 30 post-transfection. At day 3 post-transfection, only low rates of targeted T cells in the lymph nodes and the spleen are expected but the rate may increase to as much as 1% by day 10, for the lowest injected dose (1E11). The rate then declines significantly by day 30 post-injection as these mice have a full lymphocyte compartment and due to episomal vector dilution in dividing cells an in the absence of selective advantage. With intrathymic injections, little to none transduced CD19+ B cells is expected and as well as no skewing of the CD4/CD8 ratio, (expected to be 3:2 in both the eGFP− and eGFP+ subsets). Differentiated targeted cell has a naïve T cell phenotype (CD44low/CD62Lhigh) since rAAV transduction does not activate T lymphocytes leading to acquisition of an effector or memory phenotype.

### RAG Activity Assay

The experiments above are then repeated using a RAG activity reporter vector, where RAG induced recombination in cis leads to GFP expression (as illustrated in FIG. 6A). With this vector, only cells transfected as DN to ISP thymocytes initially show GFP expression as expected. The distribution of GFP+ cells within the DN subpopulations, DN1 to DN4, serves as a predictor for subsequent VDJ targeting experiments (below). Again, by day 10 post-transfection, the percentage of DP thymocytes increases subsequent to differentiation of immature transduced progenitors, however, DN transduced thymocytes are still detected at significant rates at days 10 and 30 post-injection as expected. When analyzing the migration of differentiated targeted cells to the spleen and lymph nodes, only low rates of targeted T cells are expected at day 3, but the rate may increase by day 10, and subsequently decline by day 30 post-injection due to episomal vector dilution in dividing cells and in the absence of selective advantage. The absence of transduced CD19+ B cells and a normal CD4/CD8 ratio is further validated as well as differentiated targeted cells having a naïve T cell phenotype (CD44low/CD62Lhigh).

### VDJ Targeting Reporter Assay

Next, the experiments above are repeated using a GFP reporter of VDJ targeting in the T lineage. The initial distribution of GFP+ cells reflects the integration accessibility of the integration locus. In particular, when using a vector for VDJ targeting between D and J of TCRβ (similar to the one in FIG. 4B, but with a GFP gene instead of a receptor gene) initial GFP+ cells appear among DN2 thymocytes. When using a vector for VDJ targeting between V and J segments of the TCRα locus, initial GFP+ cells are observed among DP thymocytes, and when using a vector for VDJ targeting into the TCRγ or the TCRδ locus, initial GFP+ cells are observed among DN4 thymocytes. As GFP is stably integrated into the genome, it is expected to observe expression in all subsets of thymocytes and mature T cells which are later in the developmental path than the precursor where integration was facilitated. In particular, in all cases, a high increase of GFP+DP and SP (CD4+ or CD8+) with time, is expected and finding activated GFP+ T cells acquiring effector or memory phenotypes as also expected. However, GFP+ rates at all subpopulations are still expected to be low in the absence of a selective advantage or specific activation.

### VDJ Targeting of Receptor Genes

Finally, targeting of receptor genes is evaluated. All constructs include an inducible suicide gene (iCasp9, Di Stasi A, et al. The New England journal of medicine 365 (18): 1673-1683 (2011)) to specifically and quickly eliminate the targeted cells if adverse events occur. One vector encodes a human Tn-Muc1 specific TCR construct and is injected intrathymically to conditional mouse model of human MUC1-positive endometriosis. Targeted T cell precursors pass not only positive about also negative thymic selection as expected. Expression levels in thymocyte subclasses and in T cells are examined using RT-PCR and flow cytometry. Here, TCR expressing T cells are expected to be activated upon interaction with the human MUC1 tumors, and subsequently it is expected to see eradication of the tumors by pathology and immunohistochemistry.

Yet another construct encodes an anti-mouse-CD19 CAR. CAR vectors is designed to allow integration between the D and J segment of the TCRβ locus. This allows early targeting, and assuming a CAR does not provide signals for allelic exclusion, the other allele is rearranged to provide an MHC interacting TCR in order to pass through positive thymic selection. The construct also encodes a miR-181a recognition element. In some particular and non-limiting embodiments, VDJ targeting in accordance with the invention may be employed to target the miR-181a recognition element that comprises the nucleic acid sequence as denoted by SEQ ID NO. 18. miR-181a is strongly expressed in thymocytes and markedly downregulated in post-thymic T cells (Papapetrou E P, et al., J Clin Invest 119 (1): 157-168 (2009)). In particular, the auto-reactive receptor is not expressed in thymocytes subjected to negative selection but expression ensues upon maturation. As described herein before, expression levels in thymocyte subclasses and in T cells are examined using RT-PCR and flow cytometry. Here, CAR expressing T cells are expected to be activated by the CD19 B cell antigen, leading to expansion of the targeted T cell population concomitant to elimination of natural B cells. Conversely, upon administration of the Caspase9 dimerization inducers, the targeted T cells are expected to be eliminated and the B cell compartment to be re-established through differentiation of HSCs.

Intravenous rAAV Injections after Stem Cell Mobilization

It was previously shown that mobilized HSPCs can be targeted in vivo at high rates and subsequently home back to the bone marrow where they stably express the transgene [17]. Moreover, long term surviving HSPCs can be transduced and expanded in the marrow. However, previous approaches used immunogenic adenovectors and genotoxic, promiscuously integrating transposons. Here, in vivo VDJ targeting is demonstrated after tail vein vector injections following bone marrow mobilization, without relying on myeloablative conditioning. Subcutaneous injections of G-CSF/AMD3100 is used to mobilize HSPCs from the bone marrow into the peripheral blood stream. Upon mobilization (followed by flow cytometry), tail vein injections of 1E11 to 1E12 rAAV vectors of serotypes 6, 8 and HSC derived rAAV are performed.

### Transfection Positive Control

First, canonical, promoter-driven, GFP-coding constructs are injected as a positive control, and transfection rates are estimated using RT-PCR and flow cytometry. GFP+ cells are expected among HSCs and many classes of their derived lineages. When comparing different serotypes one can determine which one allows the best transfection of B cell precursors (Pro-B, Pre-B and immature B). In lack of integration and selective advantage, the rate of GFP+ mature B cells is expected to have a naïve phenotype and their rate to rise initially but then soon decline. Homing of transduced cells back to the bone marrow is validated.

### RAG Activity Assay

Next, the experiments above are repeated using a RAG activity reporter vector, where RAG induced recombination in cis leads to GFP expression (FIG. 6A). With this vector, only cells transfected as pre-B cells to immature B cells show GFP expression. The distribution of GFP+ cells within the precursor subpopulations serves as a predictor for subsequent VDJ targeting experiments (below). Again, the percentage of mature B cells increases following differentiation of immature transduced progenitors, however, GFP+ precursors are still detected at significant rates. The rate if GFP+ B cells then declines due to episomal vector dilution in dividing cells and in the absence of selective advantage. Differentiated targeted cells are again validated for having a naïve phenotype.

### VDJ Targeting Reporter Assay:

Next, the experiments above are repeated using a GFP reporter of VDJ targeting in the B lineage. The initial distribution of GFP+ cells reflects the integration accessibility of the integration locus. In particular, when using a vector for VDJ targeting between V and J of the heavy chain (FIG. 5A, but with a GFP gene instead of an Antibody gene), initial GFP+ cells are observed among pre-B precursors. When using a vector for VDJ targeting between V and J segments of light chain locus, initial GFP+ cells are expected among immature B cells. As GFP is stably integrated into the genome, expression is observed in all subsets of the B cells lineage and mature B cells which are later in the developmental path than the precursor where integration was facilitated. In all cases, a high increase of GFP+ immature and mature B cells is expected with time, as well as activated GFP+ B cells and plasma cells acquiring effector or memory phenotypes. However, GFP+ rates at all subpopulations are still expected to be low in the absence of a selective advantage or specific activation.

### VDJ Targeting of Antibody Genes

Finally, targeting of Antibody genes is demonstrated. All constructs include an inducible suicide gene (iCasp9) to specifically and quickly eliminating the targeted cells if adverse events occur. The vector encodes PankoMab-GEX, an anti MUC1 glycoform Antibody that is highly cancer specific, and is injected intravenously to conditional mouse model of human MUC1-positive endometriosis. Targeted B cell precursors pass not only positive about also negative selection in the marrow as expected. Expression levels in B cell precursor subclasses and in mature B cells are assessed using RT-PCR and flow cytometry. Here, BCR expressing B cells are activated upon interaction with the human MUC1 tumours. Repertoire sequencing is used to monitor selective expansion and affinity maturation (somatic hypermutations as well as class switching) and subsequent eradication of the tumours is evaluated by pathology and immunohistochemistry.

Alternatively, when the mouse cancer does not express the human MUC1 glycoform that is targeted by the PankoMab-GEX Antibody, another vector is used, coding for the c-SN6j Antibody (anti-CD105). The vector is injected intravenously to a transgenic mouse model expressing relevant human CD105 epitopes on the vasculature of tumours induced by injections of 4T1 cells.

### Confronting Positive Selection

Developing T cells targeted with a transgenic TCR are positively selected in the thymus, and developing B cells targeted with a transgenic Antibody express a BCR and thus are positively selected in the bone marrow. This is true whether the lymphocytes are targeted ex vivo and subsequently transplanted or whether targeting is achieved by direct in vivo injection of the vector. However, developing T cells targeted with a CAR gene and developing B cells targeted with scFv/BiTE pass positive selection, only if allelic exclusion is absent or incomplete in the case CARs and scFv/BiTEs do not provide all the natural ques for allelic exclusion.

Alternatively, when allelic exclusion is nevertheless found to be complete, then VDJ targeting of CAR and scFv/BiTE genes is accomplished by transduction of ex vivo differentiating precursors. Targeted cells further differentiated ex vivo pass the positive selection checkpoint prior to administration.

### Confronting Negative Selection and Peripheral Tolerance

First, T cells and B cells are targeted with respective receptors and Antibodies against foreign antigens or neoantigen. Thus, the developing lymphocytes pass thymic negative selection. However, VDJ targeting can be used to engineer lymphocytes against the many common immunotherapy targets that are self-expressed antigens. When expressing a CAR/TCR against an auto-antigen, a miR-181a recognition element is incorporated into the cassette. miR-181a is highly expressed in developing T cells and markedly downregulated in post-thymic T cells. Inclusion of miR-181a recognition element can thus segregate expression of CARs and TCRs between developing and mature T cells. In particular, the auto-reactive receptor is not expressed in thymocytes subjected to negative selection but its expression is re-constituted upon maturation. Efficacy against the desired auto-antigen comes also with the risk of sustained adverse auto-immunity. Therefore, when targeting a receptor against an autoantigen along with a miR-181a recognition element, an inducible suicide gene is also included, to allow specific elimination of targeted cells. Finally, when applying VDJ targeting to ex vivo differentiating cells, cells can be differentiated past the negative selection checkpoint before being injected, and co-delivering an inducible suicide gene is again preferable.

### Example 5

### Engineering Inducible B Cells

Many monoclonal Antibodies in clinical use are targeting self-antigens. However, monoclonal Antibodies have a limited half-life, requiring repeat administration at an extremely high cost. They are prone to anti-drug Antibodies and are rarely curative. In order to engineer B cells targeting self-antigens, it is not enough to bypass central tolerance. A B cell will not be activated by an auto-antigen even if the B cell expresses a relevant BCR, because the B cell will not get the necessary help from T cells at the germinal centre. Therefore, VDJ targeting application is specifically tailored to allow safe engineering of B cells to inducibly express autoantibodies. The design as schematically represented in FIG. 20, allows targeted developing B cells to pass positive and negative selection in the bone marrow and it allows B cell activation and secretion of the autoantibody only upon induction by a foreign inducer.

The inventors target the integration of a cassette separately coding for a secreted Antibody and a BCR targeting different antigens. The secreted Antibody is directed against a disease associated Auto-antigen, while the BCR is directed against a foreign inducer. The B cell differentiates to Antibody-secreting plasma cells only upon administration of the foreign inducer. The secreted Antibody is specifically a scFv or a BiTE, rather than a full IgL-2A-IgH Antibody, in order to prevent cross pairing with the BCR chains. The BCR gene is coded in a way that prevents its secretion by avoiding the alternative splice and polyadenylation sites that are used in the natural setting for exclusion of the transmembrane domain upon activation. The foreign inducer of BCR activation is specifically an antigen of an orally administered vaccine. A prominent example is the Polio vaccine, a safe, cheap, orally administered immunogen, that is rarely encountered through the natural life course, thus diminishing the risk of spurious activation. An appropriate BCR to be activated by the polio vaccine can be designed based on well characterized human anti-poliovirus antibodies [18]. Activation is dose dependent and self-limiting thus minimizing the risk of sustained adverse auto-immunity, while allowing oral re-dosing at will. In addition, immune memory from first activation increases the potency and shorten the kinetics of subsequent activations. In different patients, different auto-antigens can be targeted for the treatment of diverse clinical indications, but the inducer can be one and the same. From the protein therapy perspective: dozens of different monoclonal antibodies are replaced by a single orally administered immunogen. Inducible efficacy of targeted B cells may be demonstrated in immunocompetent C57BL/6 mice transplanted with a syngeneic B16F10/human-Ep-CAM cells as a subcutaneous solid tumour model.

### Tumor Eradication by In Vitro Engineered Inducible-B Cells

First, VDJ targeting is performed in in vitro differentiating mouse B cells. The integrated cassette express an anti-human-Ep-CAM BiTE that is also specific to the mouse CD3 for mouse T cell engagement. In particular, VDJ targeting is used to introduce a cassette encoding for an anti-human-EpCAM BiTE and a BCR based on a human anti-polio Antibody [18]. RT-PCR is used to assess targeting rates in the different precursor subpopulations and among mature B cells, as well as flow cytometry with staining against human IgM to monitor BCR expression and presentation. ELISA is used to detect the BiTE before and after in vitro activation, to validate inducer dependent antibody secretion.

Next, the targeted B cells are implanted in C57BL/6 mice bearing syngeneic B16F10/human-Ep-CAM tumours, and in control tumour-free mice. Plasma BiTE levels are monitored by ELISA and are expected to be low in both groups, because activation relies on a polio immunogen. The Polio vaccine inducer is then orally administered and BiTE pharmacokinetics is examined by ELISA as well as dose dependent effects on tumour progression and eradication. ELISA monitoring is continued in order to assess the self-limiting nature of the induced activation, and repeat dosing are also performed to monitor how immune memory may allow faster and more potent reaction at second administration.

### Tumour Eradication by In Vivo Engineered Inducible-B Cells

in vivo VDJ targeting is applied for inducible-B cell engineering after tail vein vector injections following bone marrow mobilization, without relying on myeloablative conditioning. Subcutaneous injections of G-CSF/AMD3100 are used to mobilize HSPCs from the bone marrow into the peripheral blood stream. Upon mobilization (followed by flow cytometry), tail vein injections of 1E11 to 1E12 rAAV vectors (of serotypes 6, 8 and HSC derived rAAV) are provided. The integrated cassette again encodes an anti-human-EpCAM BiTE and a BCR based on a human anti-polio Antibody [18]. The presence of mature B cells expressing human IgM, monitored by flow cytometry, attests that the targeted precursors have homed back to the bone marrow and differentiated, passing both positive and negative selection. As described above, plasma BiTE levels are monitored by ELISA and are expected to be low, because activation relies on a polio immunogen. The Polio vaccine inducer is then administered orally, and BiTE pharmacokinetics are analysed by ELISA as well as dose dependent effects on tumour progression and eradication. ELISA monitoring is continued in order to validate self-limiting activation, and repeat dosing is also performed for monitoring how immune memory may allow faster and more potent reaction at second administration.

1. A method for treating, preventing, ameliorating, inhibiting or delaying the onset of a pathologic disorder in a mammalian subject, said method comprising the step of administering to said subject an effective amount of at least one of:
(a) nucleic acid cassette;
(b) a vector comprising said nucleic acid cassette; and
(c) a cell transduced or transfected with said nucleic acid cassette;
wherein said cassette comprises at least one nucleic acid sequence of interest and at least one RSS, wherein the cassette is for targeted insertion of at least one nucleic acid sequence of interest into a target genomic locus, and wherein the insertion of said nucleic acid sequence of interest into the target genomic locus is mediated by RAG-catalyzed recombination between at least one genomic RSS flanking the target genomic locus and at least one RSS comprised within said nucleic acid cassette.

2. The method according to claim 1, wherein said subject undergoes a preceding treatment of at least one of HSPC mobilization, immune ablation and steroid administration.

3. The method according to claim 1, wherein said nucleic acid sequence of interest is a protein coding nucleic acid sequence or a non-coding nucleic acid sequence.

4. The method according to claim 3, wherein said nucleic acid sequence of interest encodes at least one of a receptor and an antibody or any fragment/s or chimera/s thereof.

5. The method according to claim 1, wherein said target genomic locus is at least one of Immunoglobulin heavy chain locus, Immunoglobulin κ chain locus, Immunoglobulin λ chain locus, TCRβ chain locus, TCRα chain locus, TCRγ chain and the TCRδ chain locus.

6. The method according to claim 1, wherein said subject is administered with a nucleic acid vector comprising said cassette, said vector is any one of a viral vector, a non-viral vector and a naked DNA vector.

7. The method according to claim 6, wherein said nucleic acid vector is administered by at least one of systemic injection, intrathymic injection, bone marrow injection, splenic injection and lymph node injection.

8. The method according to claim 1, wherein said subject is administered with at least one cell transduced or transfected with said nucleic acid cassette or any vector or vehicle comprising said cassette.

9. The method according to claim 8, wherein said cells are of an autologous or allogeneic source.

10. The method according to claim 1, wherein said subject is a subject suffering of an immune-related disorder.

11. A nucleic acid cassette comprising at least one nucleic acid sequence of interest and at least one RSS.

12. The cassette according to claim 11, wherein said cassette is for targeted insertion of at least one nucleic acid sequence of interest into a target genomic locus in a mammalian cell, wherein the insertion of said nucleic acid sequence of interest into the target genomic locus is mediated by RAG-catalyzed recombination between at least one genomic RSS flanking the target genomic locus and at least one RSS comprised within said nucleic acid cassette.

13. The nucleic acid cassette according to claim 11, wherein said cassette comprises two or more nucleic acid sequences of interest separated by at least one genetic element, said genetic element is at least one of: an IRES, a 2A peptide coding sequence, a promoter or any functional fragments thereof, a splice donor (SD), a splice acceptor (SA).

14. The nucleic acid cassette according to claim 11, wherein said nucleic acid sequence of interest encodes at least one of a receptor and an antibody or any fragment/s or chimera/s thereof.

15. The nucleic acid cassette according to claim 11, wherein said target genomic locus is at least one of Immunoglobulin heavy chain locus, Immunoglobulin κ chain locus, Immunoglobulin λ chain locus, TCRβ chain locus, TCRα chain locus, TCRγ chain and the TCRδ chain locus.

16. The nucleic acid cassette according to claim 11, wherein said cassette is comprised within a nucleic acid vector, said vector is any one of a viral vector, a non-viral vector and a naked DNA vector.

17. A pharmaceutical composition comprising at least one nucleic acid cassette, or any vector or cell comprising said cassette, wherein said cassette comprises at least one nucleic acid sequence of interest and at least one RSS, said cassette is for targeted insertion of said at least one nucleic acid sequence of interest into a target genomic locus, wherein the insertion of said nucleic acid sequence of interest into the target genomic locus is mediated by RAG-catalyzed recombination between at least one genomic RSS flanking the target genomic locus and at least one RSS comprised within said nucleic acid cassette, optionally said composition further comprise at least one of pharmaceutically acceptable carrier/s, diluent/s, excipient/s and additive/s.

18. A cell or a population of cells transduced or transfected with the nucleic acid cassette according to claim 11; wherein said cassette comprises at least one nucleic acid sequence of interest and at least one RSS, wherein the cassette is for targeted insertion of at least one nucleic acid sequence of interest into a target genomic locus, and wherein the insertion of said nucleic acid sequence of interest into the target genomic locus is mediated by RAG-catalyzed recombination between at least one genomic RSS flanking the target genomic locus and at least one RSS comprised within said nucleic acid cassette.

19. The cell or a population of cells according to claim 18, wherein said cells are of an autologous or allogeneic source.

20. The cell or a population of cells according to claim 19, wherein said cell is of a subject suffering of an immune-related disorder.
